(19) United States
(12) Patent Application Publication
(10) Pub. No.: US 2012/0034221 A1
(43) Pub. Date: Feb. 9, 2012

(54) T-Cell Receptor Antibodies And Methods of Use Thereof

(75) Inventors: Ezio Bonvini, Rockville, MD (US); Leslie S. Johnson, Damestown, MD (US); Nadine Tuaillon, Gettysburg, PA (US); Ling Huang, Bethesda, MD (US)

(73) Assignee: MACROGENICS INC., Rockville, MD (US)

(21) Appl. No.: 13/273,282

(22) Filed: Oct. 14, 2011

Related U.S. Application Data
(63) Continuation of application No. 13/060,460.
(60) Provisional application No. 61/092,005, filed on Aug. 26, 2008.

(51) Int. Cl. A61K 39/395; C12N 15/13; C12N 15/63; C12P 21/02; C12N 1/21; C12N 5/10; C12N 1/19; A61P 37/06; A61P 35/00; A61P 29/00; A61P 3/10; A61P 17/06; A61P 1/00; A61P 25/00; C07K 16/28

(52) U.S. Cl. 424/133.1; 530/389.6; 530/387.3; 424/173.1; 536/23.53; 435/320.1; 435/69.6; 435/252.33; 435/334; 435/419; 435/254.2; 435/252.1

(57) ABSTRACT

The present invention is directed to the production and use of monoclonal antibodies, or antigen binding fragments thereof, that specifically bind the T cell antigen receptor (TCR) and their use for immunomodulation. In preferred embodiments, the antibody or antigen binding fragment of the invention specifically binds the constant region of the α chain of the TCR, or otherwise specifically binds the α chain regardless of TCR clonal origin (i.e., is pan specific for TCR). The antibodies of the invention may be used, for example, in immunosuppressive therapies for transplant maintenance and the treatment of autoimmune diseases, and/or as targeting molecules for use in the treatment of T-cell malignancies.

This application is a continuation under 35 U.S.C. §120 of application Ser. No. 13/060,460, filed Feb. 24, 2011, which is the entry of the national phase under 35 U.S.C. §371 of PCT/US09/54911, filed Aug. 25, 2009, which claims benefit of priority to U.S. provisional application No. 61/092,005, filed on Aug. 26, 2008, the contents of each of which are incorporated herein in their entirety.

### 1. INTRODUCTION

The present invention is directed to the production and use of monoclonal antibodies, or antigen binding fragments thereof, that specifically bind the T cell antigen receptor (TCR) and their use for immunomodulation. In preferred embodiments, the antibody or antigen binding fragment of the invention specifically binds the constant region of the α chain of the TCR, or otherwise specifically binds the α chain regardless of TCR clonal origin (i.e., is pan specific for TCR). The antibodies of the invention may be used, for example, in immunosuppressive therapies for transplant maintenance and the treatment of autoimmune diseases, and/or as targeting molecules for use in the treatment of T-cell malignancies.

### 2. Background of the Invention

2.1 T Cells and the T Cell Antigen Receptor

T lymphocytes (also known as T cells) are grouped into various subsets generally defined by antigenic determinants found on their surface as well as differences in functional activity and antigen recognition ability. For example, CD8+ T cells are recognized generally as cytotoxic T cells that function to identify and kill foreign cells or host cells displaying foreign antigens, while CD4+ T cells are classified as helper cells that promote humoral and inflammatory responses by increasing the avidity between the T cell and its stimulator cells.

T cell activity is regulated by the binding of any number of ligands to their respective receptors on the T cells surface. Where such binding results in classic T cell activity (e.g., increased production of lymphokines or cytokines, cytotoxic cell activity, proliferation) the T cell is said to have been activated. Of particular interest is the activation of T cells via binding of the antigen specific T cell receptor (TCR) to antigens bound to the major histocompatibility complex (MHC) on the surface of antigen presenting cells. However, T cell activation is a complex event that depends on the participation of a variety of T cell surface molecules. For example, the antigen-specific T cell receptor (TCR) is actually a complex of a disulfide linked heterodimer, containing two clonally distributed transmembrane glycoprotein chains (α and β, or γ and δ), non covalently associated with a complex of invariant low molecular weight proteins (the CD3 complex).

The TCR α and β or γ and δ chains determine antigen specificity, resulting in highly polymorphic TCRs between T cells of different specificities. Approximately 90 percent of peripheral blood T cells express the α and β heterodimer, while only a minority have been shown to express a TCR consisting of γ and δ variety. The chains of the TCR are each composed of a unique combination of domains designated variable (V), diversity (D), joining (J), and constant (C). In each T cell clone, the combination of V, D and J domains of both the α and the β chains (or of both the γ and δ chains) participates to define a unique binding site, also known as the idiotype of the T cell clone. The C domain does not participate in antigen binding.

The tremendous heterogeneity of the TCR remains a limiting factor in the clinical application of TCR antibodies. Although antibodies have been developed that are capable of immunospecifically binding the TCR, these antibodies have proven to be of highly specific application in that they tend to be clonotypic (i.e., react only with a particular clone of T cells) and/or are clinically irrelevant. For example, Acuto et al. produced clonotypic monoclonal antibodies against a human thymocyte cell line, identifying the TCR in relatively undifferentiated T3⁺ cells (1983, Cell 34:717-726). Meuer et al. showed that anti-TCR clonotypic monoclonal antibodies coupled to sepharose beads could induce production of interleukin-2 (1984, Proc. Natl. Acad. Sci. 81:1509-1513). An anti-TCR clonotypic antibody directed toward the CT8 cell line could only block cytotoxic effector cell function of the particular T cell line (Meuer et al., 1984, Ann. Rev. Immunol. 2.23-50). Accordingly, multiple efforts have been attempted to develop pan-specific antibodies, or antibodies that recognize TCR from many T cell lines. It was initially believed that such pan-specific antibodies were impossible. Brenner et al. found that different cloned T cell lines shared antigenic determinants but that none appeared to be accessible at the cell surface (1984, J. Exp. Med. 160:541-551). Although shared determinants theoretically on the cell surface were discovered, antibodies directed to these epitopes proved of limited use. Brenner et al, produced a β-Framework-1 (βF1) monoclonal antibody that reacts with such an epitope, but it was found to bind a “hidden determinant” on the surface of viable T cells, recognizing the TCR β polypeptide only in Western blots (1987, J. Immunol. 138:1502-1509). Another antibody, WT31, originally thought to bind a framework region of the TCR was found useful in cell binding assays, but was found inefficient for immunoprecipitation (Spits et al., 1985, J. Immunol. 135:1922-1928). Further, WT31 was found specifically to recognize a CD3 determinant. In contrast, the BMA 031 antibody represents unique clinical opportunities because it is pan-specific for TCR on mature, immunocompetent T cells. BMA 031 recognizes a determinant of the polymorphic α/β-TCR with no cross reactivity with the more premature γ/δ-T cell population (Jisukawa et al., 1987, J Exp Med 166:1192-1197; Faure et al., 1988, J Immunol 140:2128-2132). Accordingly, in specific embodiments, the instant application is directed to pan-specific anti-TCR antibodies. (Various publications are cited in the preceding paragraph, each of which is hereby incorporated by reference in their entirety).

2.2 Autoimmune Diseases

Autoimmune diseases are caused when the body's immune system, which normally defends the body against bacteria, viruses and other infective agents, attacks “self’ tissue, cells and organs. The mobilization of the immune system against such self targets is termed autoimmunity. Although some autoimmunity is present in every individual, rigid control systems suppress the self-recognizing cells of the immune system to an extent that the autoimmunity is normally asymptomatic. Disease states arise when there is some interruption in the control system, allowing the autoimmune cells to escape suppression, or when there is some change in a target tissue such that it is no longer recognized as self. The mechanisms underlying these changes are not well understood, but have been theorized to be the result of aberrant immune stimulation in genetically predisposed individuals.

Autoimmune diseases can be organ specific or systemic and are provoked by differing pathogenic mechanisms. Organ specific autoimmunization is characterized by tolerance and suppression within the T cell compartment, aberrant expression of major-histocompatibility complex (MHC) antigens, antigenic mimicry and allelic variations in MHC genes. Systemic autoimmune diseases usually involve polyclonal B cell activation and abnormalities of immunoregulatory T cells, T cell receptors and MHC genes. Examples of organ specific autoimmune diseases are diabetes, cutaneous psoriasis, ulcerative colitis, hyperthyroidism, autoimmune adrenal insufficiency, hemolytic anemia, multiple sclerosis and rheumatic carditis. Representative systemic autoimmune diseases include systemic lupus, erythematosus, rheumatoid arthritis, psoriatic arthritis, Sjogren's syndrome polymyositis, dermatomyositis and scleroderma.

Also, while not having an autoimmune disorder, organ transplant recipients often experience similar symptoms and require similar therapies to autoimmune patients. Immune system attacks on the transplanted organ(s) can lead to organ failure or more serious systemic complications, e.g., graft-vs.-host disease (GVHD) in bone-marrow transplant recipients.

There is a clear need for improved strategies to treat autoimmune disorders and/or to modulate immune response. Currently, immune system disorders are treated with immunosuppressive agents such as cortisone, aspirin derivatives, hydroxychloroquine, methotrexate, azathioprine, cyclophosphamide and various biologics such as anti TNF antibodies, and/or combinations of the foregoing. The treatments are varyingly successful, dependent on the individual patient and disorder. However, a dilemma in the use of such general immunosuppressive therapies arises in that the greater the immune-suppression, and thus the increased potential for successful treatment of the autoimmune disorder, the more at-risk the patient becomes for developing opportunistic infections. Further, due to the compromised nature of the patient's immune system, even a minor infection can rapidly become of serious concern.

2.3 T cell Functionality in Autoimmune Disorders

Destruction of β-cells in diabetes, of myelin in multiple sclerosis, or of the target cells of other autoimmune disorders is believed largely mediated by cytotoxic T-lymphocytes (CTLs—also known as CD8+ T cells) that specifically recognize antigenic, target cell derived peptides. CTLs, as well as other types of T cells, recognize these antigenic peptides through their specific T cell receptor (TCR). Unlike antibodies which recognize soluble whole foreign proteins as antigen, the TCR instead interacts with small peptidic antigens presented only in complex with major histocompatibility complex (MHC) proteins.

Most cells of the body express MHC molecules of various classes on their surface and, depending on the class of MHC expressed, will present either soluble antigens, those dispersed within the lymph and/or circulatory systems, or fragments of their cytoplasmic proteins. MHC molecules (called human leukocyte antigens or HLA in humans) and TCRs are extremely polymorphic, each clonal variation recognizing and binding to a single peptidic sequence, or set of similar peptidic analogs. Apart from cells specific to the immune system, i.e. B cells and T cells, cells of the body express multiple variants of the MHC molecule, each variant binding to a different peptide sequence. In contrast, during maturation, B and T cells lose the ability to express multiple variants of MHC and TCR, respectively. Mature T cells, therefore, will express only one of the possible variants of the TCR and will thus recognize/bind a single MHC/antigen complex.

Binding of a TCR to a MHC/antigen complex elicits an intracellular signal cascade within the T cell, termed activation, which results in clonal proliferation of the T cell and class-specific T cell responses. For example, in CTLs the response to activation also includes the release of cytotoxic enzymes that result in apoptosis/destruction of the target cell.

2.4 Modulation of T cell Activation by Monoclonal Antibodies

The finding that autoimmune diseases are at least partially caused by aberrant T cell action has lead to the investigation of therapies that either eliminate problematic T cell clones (those expressing TCRs against self antigens) or selectively reduce undesired T cell activity/activation. T cell activation due to TCR binding is, however, an unexpectedly complex phenomenon due to the participation of a variety of cell surface molecules expressed on the responding T cell population (Billadeau et al., 2002, J. Clin. Invest. 109:161-168; Weiss, 1990, J. Clin. Invest. 86:1015-1022; Leo et al., 1987, PNAS 84:1374-1378; Weiss et al., 1984, PNAS 81:4169-4173; Hoffman et al., 1985, J. Immunol. 135:5-8).

Targeted therapies directed against general T cell activation were problematic in that the TCR is composed of a disulfide-linked heterodimer, containing two clonally distributed, integral membrane glycoprotein chains, α and β, or γ and δ. Most of the research in modulation of T cell activation was done in connection with improving immune suppression in organ transplant recipients. However, antibodies to the TCR α/β chain have shown therapeutic efficacy in acute models of experimental autoimmune encephalomyelitis (EAE) and arthritis (see, e.g., Masumoto et al., 1994, Immunology 81:1-7; Goldschmidt et al., 1991, Eur J Immunol 21:1327-1330; Yoshino et al., 1992, J Exp Med 175:907-915, each incorporated by reference in their entirety), although the effects of anti-TCR antibody are unclear. Generally, the therapeutic effects are attributed to T-cell depletion of functional blocking of T-cells involved in the autoimmune reaction, but studies have indicated the TCR signaling and/or T-cell activation may be necessary for therapeutic effect (Goldschmidt et al., 1991, Eur J Immunol 21:1327-1330, incorporated by reference in its entirety).

2.5 Immunosuppressive Monoclonal Antibodies Exhibiting Reduced T Cell Activation

Use of immunomodulatory antibodies directed against T-cells and/or components of the TCR are often hampered by a toxic “first dose response.” The first dose response has been shown to be related to the activation of the T-cells and ensuing release of cytokines prior to suppression of the T-cell response and/or activation. The primary activating properties of the antibodies causing first dose responses are believed to be mediated by the cross-linking of T-cells to Fc receptor (FcR) bearing cells via the antibody Fc domain. The resulting cross-linking activates both types of cells, resulting in a “cytokine storm,” a massive systemic release of cytokines Experiments using F(ab′)₂ fragments and/or antibodies having Fc regions exhibiting reduced or abrogated FcR binding have demonstrated that eliminating Fc binding effectively mediates the adverse side effects of anti-T-cell antibodies without compromising their immunomodulatory properties. Several methods are known in the art to decrease or abrogate binding of the Fc domain of an antibody to an FcR. For example, U.S. Pat. No. 6,491,916, U.S. Pat. Application Pub. No. 2005/0064514 and U.S. Pat. Application Pub. No. 2005/0037000 describe the modification of the Fc regions of immunoglobulins such that the variant molecules exhibit enhanced or reduced binding to various Fc receptors when compared to immunoglobulins with wild type Fc domains. In particular the patents/applications describe modifications to the Fc regions of IgG antibodies such that the affinity for the FcγR is selectively enhanced or reduced. By tailoring the affinity for activating or suppressive Fc receptors, the specific immune response elicited by the therapeutic mAb may be more selectively controlled. For example, mutations in the CH₂ portion of a humanized antibody have been identified (P234A and L235A) that significantly reduced binding of the mAb to human and murine FcγRI and II and lead to a markedly reduced activating phenotype relative to control in vitro. Importantly, this variant mAb retained the capacity to induce TcR modulation and immunosuppression. Other modifications to the Fc domain of anti-CD3 antibodies, such as mutations to make the antibody aglycosylated or other mutations of the Fc domain residues, to reduce binding to FcγR have been found to reduce toxicity while maintaining immunosuppressive activity (see, e.g., U.S. Pat. No. 6,491,916; U.S. Pat. No. 5,834,597, Keymeulen et al., 2005, N. Eng. J. Med. 325:2598, all of which are incorporated by reference herein in their entireties).

### 3. SUMMARY OF THE INVENTION

The present invention is directed to isolated monoclonal antibodies, or antigen binding fragments thereof, that specifically bind the T cell antigen receptor (TCR). The antibodies of the invention may be used for immunomodulation in a patient in need thereof, for example, in immunosuppressive therapies in organ and bone transplantation and the treatment of autoimmune diseases, and/or as targeting molecules for the treatment T-cell malignancies.

The antibodies or antigen binding fragments thereof may bind one or more of the polypeptide chains forming the TCR, including the α, β, γ and/or δ chain, and may bind one or more regions of said chains, including the variable, diversity, joining and/or constant region. The antibodies of the invention may be clonotypic, in that they specifically bind a single clone of TCR (i.e., a defined subset of TCR expressed within a T cell population) or any subset of T-cells, or, in preferred embodiments, may specifically react with the TCR regardless of clonotypic origin. The antibody of the invention may immunospecifically bind the α, β, γ or δ chain of the TCR as part of an intact TCR complex, or may bind the α, β, γ or δ chain as an individual peptide or fragment thereof. In preferred embodiments, the antibody of the invention, or an antigen binding fragment thereof, specifically binds the α chain of TCR and, more preferably, binds the constant and/or extracellular domain of the α chain of TCR. In other embodiments, the antibody of the invention, or an antigen binding fragment thereof, specifically binds the β chain of TCR and, preferably, binds the constant and/or extracellular domain of the β chain of TCR. In yet other embodiments, the antibody of the invention, or an antigen binding fragment thereof, specifically binds the γ chain of TCR and, preferably, binds the constant and/or extracellular domain of the γ chain of TCR. In still other embodiments, the antibody of the invention, or an antigen binding fragment thereof, specifically binds the δ chain of TCR and, preferably, binds the constant and/or extracellular domain of the δ chain of TCR.

In preferred embodiments, the invention provides an antibody, or antigen binding fragment thereof, that specifically binds the constant region of the α chain of the TCR or otherwise specifically binds the α chain regardless of clonal origin of the T-cell; in such embodiments, the antibody of the invention recognizes TCR α chains, and, thus the α/β TCR, generally (i.e., is a pan-specific TCR antibody, and, in particular, a pan-specific TCR α chain antibody). In other embodiments, the invention provides an antibody, or antigen binding fragment thereof, that specifically binds the constant region of the β chain of the TCR or otherwise specifically binds the β chain regardless of clonal origin of the T-cell; in such embodiments, the antibody of the invention recognizes TCR β chains, and, thus the α/β TCR, generally (i.e., is a pan-specific TCR antibody, and, in particular, a pan-specific TCR (3 chain antibody). In yet other embodiments, the invention provides an antibody, or antigen binding fragment thereof, that specifically binds the constant region of the γ chain of the TCR or otherwise specifically binds the γ chain regardless of clonal origin of the T-cell; in such embodiments, the antibody of the invention recognizes TCR γ chains, and, thus the γ/δ TCR, generally (i.e., is a pan-specific TCR antibody, and, in particular, a pan-specific TCR γ chain antibody). In still other embodiments, the invention provides an antibody, or antigen binding fragment thereof, that specifically binds the constant region of the δ chain of the TCR or otherwise specifically binds the δ chain regardless of clonal origin of the T-cell; in such embodiments, the antibody of the invention recognizes TCR δ chains, and, thus the γ/δ TCR, generally (i.e., is a pan-specific TCR antibody, and, in particular, a pan-specific TCR δ chain antibody).

In a specific embodiment, the invention provides an antibody, or antigen binding fragment thereof, that is pan-specific for the TCR α chain. In a specific example in accordance with this embodiment, the antibody of the invention comprises a heavy or light chain variable domain derived from the heavy or light chain of the murine antibody BMA 031, as set forth in SEQ ID NO:1 (FIG. 1) and SEQ ID NO:7 (FIG. 2), respectively, and/or a derivative or variant thereof. In other embodiments, the antibody of the invention comprises one or more of a heavy chain CDR1 having the amino acid sequence of SEQ ID NO:13 or SEQ ID NO:14, a heavy chain CDR2 having the amino acid sequence of SEQ ID NO:15, a heavy chain CDR3 having the amino acid sequence of SEQ ID NO:16, a light chain CDR1 having the amino acid sequence of SEQ ID NO:17, a light chain CDR2 having the amino acid sequence of SEQ ID NO:18, a light chain CDR3 having the amino acid sequence of SEQ ID NO:19 and/or a variant or derivative thereof. In certain embodiments, the TCR binding molecule of the invention does not comprise a heavy chain variable domain having the amino acid sequence of SEQ ID NO:1 and/or a light chain variable domain having the amino acid sequence of SEQ ID NO:7. In other embodiments, the TCR binding molecule of the invention does not comprise a heavy chain CDR1 having the amino acid sequence of SEQ ID NO:13, and/or does not comprise a heavy chain CDR2 having the amino acid sequence of SEQ ID NO:15, and/or does not comprise a heavy chain CDR3 having the amino acid sequence of SEQ ID NO:16, and/or does not comprise a light chain CDR1 having the amino acid sequence of SEQ ID NO:17, and/or does not comprise a light chain CDR2 having the amino acid sequence of SEQ ID NO:18 and/or does not comprise a light chain CDR3 having the amino acid sequence of SEQ ID NO:19. In other embodiments, the antibody of the invention, of an antigen binding fragment thereof, comprises a VH domain having the amino acid sequence of SEQ ID NO:2, SEQ ID NO:3, SEQ ID NO:4, SEQ ID NO:5 or SEQ ID NO:6 (as set forth in FIG. 1) and/or a VL domain having the amino acid sequence of SEQ ID NO:8, SEQ ID NO:9, SEQ ID NO:10, SEQ ID NO:11 or SEQ ID NO:12 (as set forth in FIG. 2), or a variant or derivative thereof. In preferred embodiments, the invention comprises a heavy chain variable domain having the amino acid sequence of SEQ ID NO:6 and a light chain variable domain having the amino acid sequence of SEQ ID NO:12.

The invention also encompasses TCR binding molecules comprising an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and/or SEQ ID NO:12. In specific embodiments the TCR binding molecules of the invention does not comprise a VH, VL, and/or a CDR from BMA 031-EUCIV2, BMA 031-EUCIV3 or BMA EUCIV7 as disclosed in Shearman et al., J Immunol 147:4366-4373 (incorporated by reference herein in its entirety).

In certain embodiments, the antibodies of the invention, or antigen binding fragments thereof specifically bind TCR, e.g., bind the α, β, γ, δ chain of the TCR, preferably the α chain of the TCR, but do not bind the antigen/MHC binding site of the receptor and/or do not bind the CD3 complex. Thus, an antibody of the invention, or an antigen-binding fragment thereof, may bind complexed or uncomplexed TCR. In other embodiments, an antibody of the invention, or an antigen binding fragment thereof, may only bind only a complexed TCR. In alternate embodiments, an antibody of the invention, or an antigen binding fragment thereof, may only bind only an uncomplexed TCR. In related embodiments, the antibodies of the invention, or antigen binding fragments thereof, specifically bind uncomplexed TCR and interfere with or block the binding and/or complexing of the TCR to its ligand (i.e., an antigen/MHC complex).

The invention encompasses antibodies, or antigen binding fragments thereof, that specifically bind TCR and modulate receptor activity, effecting immunomodulation by modulating the activity of the T-cell expressing said TCR. In certain embodiments, the antibodies of the invention, or antigen binding fragments thereof, agonize at least one activity of TCR, i.e., elicit signaling. As is well known in the art, TCR signaling can lead to T-cell activation, including increased cell proliferation and the increased expression of cytokines and cytokine receptors (e.g., IL-2 and IL-2 receptors). In a specific example in accordance with this embodiment, an antibody of the invention, or an antigen binding fragment thereof, may bind complexed TCR and increase receptor signaling relative to that in the absence of the antibody of the invention, leading to an enhanced immune response. In other embodiments, the antibodies of the invention, or antigen binding fragments thereof, antagonize at least one activity of TCR, i.e., inhibit signaling. In a specific example in accordance with this embodiment, an antibody of the invention, or an antigen binding fragment thereof, may bind complexed TCR and decrease receptor signaling relative to that in the absence of the antibody of the invention, leading to a decreased immune response.

In certain embodiments, the antibodies of the invention, or antigen binding fragments thereof, block the ligand binding site of TCR. In other embodiments, the antibodies of the invention bind to the TCR and inhibit or abolish the ability of the receptor to complex with CD3. In these embodiments, the blocking activity can block the immune-complex-triggered activation of the T-cell expressing said TCR and consequently suppress immune response. In another specific embodiment, the antibodies of the invention, or antigen binding fragments thereof, block crosslinking of T-cells to APCs (e.g., dendritic cells, B cells, macrophages etc.), leading to immune suppression.

The antibodies of the invention can be used to deplete T cells and/or to inhibit T cell activation in vivo in mammals, including humans. Therapeutic regimens can be designed in which antibodies are administered, using standard methods, in order to inhibit antigen recognition, by binding to T cell surface TCR and thereby sterically blocking the interaction between the variable region of the TCR and the specific complex of antigenic peptide and MHC molecule. Alternatively, or in addition, the complexes formed between the TCR-specific antibodies and the cell surface TCR can deplete undesired T cells, e.g., aberrant T cells associated with a leukemia or lymphoma, by utilizing accessory elements of the immune system that destroy the antibody-bound T cell. In such embodiments, it is anticipated that the Fc region of antibodies bound to TCR on the T cell surface will engage and activate cytotoxic mechanisms mediated by the complement system, macrophages, monocytes, or antibodydependent cytotoxic cells. Accordingly, in such embodiments, the binding of the Fc region to components of the immune system, e.g., the complement system or a FcR, may be increased by modification of the Fc region using methods well known in the art. The efficiency of T cell depletion may also be enhanced by administering TCR-specific antibodies that are covalently conjugated to a cytotoxic or anti-metabolic agent, such as toxins of microbial or synthetic origin, including peptide toxins or polypeptides related to toxins, enzymes, radioactive substances, or cytotoxic drugs. In applications of TCR specific antibodies in vivo as immune response modifiers (e.g., immunoregulators, immunosuppressors, immunoactivators), the selection of antibodies with defined specificity allows targeting of either the whole T cell population, or a defined T cell sub-population, within an individual animal or human. For example, antibodies specific for a clonotypic epitope would target only the members of a single T cell clonotype, whereas antibodies specific for family-specific epitope (e.g., variable α, β, γ, or δ chain) would target all the T cell clones having TCRs using the epitope bearing segments, which would belonging to a particular family. Only those T cells involved in a particular disease or medically undesirable immune response would be targeted for modulation or elimination; the majority of T cells involved in the maintenance of immunity against infectious agents would be spared. Antibodies can be administered directly; alternatively, they can be administered indirectly, such as by maternal transmission (transplacental transmission to offspring of a mammal during gestation, or by transmission during nursing).

The invention encompasses the use of anti-TCR antibodies, and/or antigen binding fragments thereof, to effect immunomodulation in a subject in need thereof. In preferred embodiments, the subject is a mammal, and more preferably, a human. The methods of the invention encompass immunomodulation in a subject in need thereof for a variety of purposes that will be readily ascertainable to one of skill in the art. Examples of such uses include, but are not limited to, general or specific (i.e., directed to only a subset of T-cells) immunosuppression during transplant procedures (e.g., hematopoietic tissue or organ transplant). In other embodiments, the antibody of the invention, or an antigen binding fragment thereof, may be used in the treatment or management of an autoimmune disease, an infection, cancer or other malignancy or immunodeficiency. Immunomodulation refers to any treatment course that alters the immune system by suppressing or enhancing the immune system. Accordingly, immunosuppression and immunostimulation are subsets of immunomodulation.

As is well known in the art, the use of therapeutic antibodies directed to cells of the immune system may be limited by problems of “first dose” side effects. First dose side effects, range from mild flu-like symptoms to severe toxicity, can be mild to severe, and include symptoms, such as, high fever, chills/rigors, headache, tremor, nausea/vomiting, diarrhea, abdominal pain, malaise, muscle/joint aches and pains, and generalized weakness. The first dose side effects are believed to be caused by lymphokine production and cytokine release stimulated by the Fc region of an antibody binding to and activating an FcγR on an FcγR-expressing cell. The invention thus provides TCR antibodies that exhibit reduced or abrogated first dose side effects, or exhibit at least one reduced or abrogated symptom associated with first dose side effects. Antibodies that exhibit abrogated or reduced Fc effector function may find particular use in therapeutic applications wherein Fc region-mediated immune responses are not desired or are unnecessary (e.g., use of the antibodies of the invention to inhibit the interaction of the TCR and the target antigen/MHC complex, e.g., in the treatment of an autoimmune disease). In certain embodiments, the antibodies of the invention reduce or abrogate first dose effects, or one or more symptoms thereof, in that they do not comprise an immunoglobulin Fc region (e.g., is a Fab fragment, F(ab′)₂ fragment or single chain antibody). In other embodiments, the antibodies of the invention reduce or abrogate first dose effects, or one or more symptoms thereof, in that they comprise an Fc domain that exhibits reduced or no detectable binding to one or more effector ligands. The Fc effector ligand can be any FcR (e.g., FcγR), or any component of complement (e.g., C1q). In one example in accordance with this embodiment, the antibody of the invention comprises a variant Fc region having one or more amino acid modifications, relative to a wild-type Fc region. The one or more modifications decrease or eliminate binding of the Fc to one or more FcγRs or to one or more components of complement, relative to a comparable wild-type Fc region. The one or more modifications are typically amino acid substitutions, but can also be amino acid insertions or deletions, or any combination of amino acid insertion, deletion and substitution. Modification of Fc domain binding to FcR may comprise modification of the Fc CH2 and/or hinge region. Alternatively, binding of Fc to one or more effector ligands can be reduced or eliminated by altering or eliminating one or more glycosyl groups in one or more Fc regions. Fc glycosylation can be altered or eliminated by methods well known in the art. For example, Fc glycosylation can be altered by producing the Fc in a cell that is deficient in fucosylation (e.g., fuc6 null cells), or eliminated by deglycosylation enzymes or an amino acid modification that alters or eliminates a glycosylation site (e.g., the N—X—S/T glycosylation site at positions 297-299 in the CH2 domain). In a specific embodiment, the invention provides an antibody comprising an Fc domain, wherein the amino acid corresponding to position 297 of the C_{H}2 domain is aglycosyl. In another embodiment, the antibody of the invention comprises an aglycosyl Fc region that has reduced binding to at least one Fc effector ligand compared to a reference antibody or protein that comprises an unmodified Fc region. In preferred embodiments, the antibody of the invention has diminished (such as, but not limited to, less than 50%, less than 40%, less than 30%, less than 20%, less than 10%, less than 5% or less than 1% as compared to binding by an antibody having a wild-type, glycosylated Fc domain) or, more preferably, no detectable binding to one or more of any FcγR (e.g., FcγRI, FcγRII or FcγRIII) via its Fc domain as determined by assays routine in the art. In addition or alternatively, the antibody of the invention has diminished (such as, but not limited to, less than 50%, less than 40%, less than 30%, less than 20%, less than 10%, less than 5% or less than 1% as compared to binding by an antibody having a wild-type, glycosylated Fc domain) or, more preferably, no detectable binding to any complement receptors, such as, C1q, as determined in routinely used assays.

In certain embodiments, the antibody of the invention comprises a variant Fc region (including an Fc derived from any human immunoglobulin type (e.g., IgG, IgE, IgM, IgD, IgA and IgY), or class (e.g., IgG₁, IgG₂, IgG₃, IgG₄, IgA₁, and IgA₂) or subclass), wherein said variant Fc region comprises at least one amino acid modification relative to a wild-type Fc region, which variant Fc region exhibits reduced or abolished binding to one or more effector ligands as determined by standard assays known in the art and disclosed herein, relative to a comparable molecule comprising the wild type Fc region. In certain embodiments, the variant Fc domain of the antibody of the invention comprises an amino acid modification (i.e., insertion, substitution, deletion) at one or more of the residues 233, 234, 235, 236, 237, 238, 265, 270, 297, 298, 299. In a specific embodiment, the one or more amino acid modifications which reduce or abolish binding to one or more effector ligands is a substitution with phenylalanine or proline at position 233; a substitution with alanine at position 234; a substitution with alanine or glutamic acid at position 235; a substitution with alanine at position 236, a substitution with alanine at position 237, a substitution with arginine at position 238; a substitution with alanine or glutamic acid at position 265; a substitution with alanine or asparagine at position 270; a substitution with alanine or glutamine at position 297; a substitution with phenylalanine, asparagine or proline at position 298; a substitution with any amino acid at position 299 other than serine or threonine; or a combination of two or more of the above-listed substitutions. In certain embodiments, the antibody of the invention comprises an Fc domain having a substitution with alanine at position 265 and at position 297; a substitution with alanine at position 265 and with glutamine at position 297; a substitution with glutamic acid at position 265 and with alanine at position 297; or a substitution with glutamic acid at position 265 and with glutamine at position 297. In preferred embodiments, the antibody of the invention comprises an Fc domain having a modification (e.g., substitution, insertion, deletion) at position 234 and position 235 of the Fc region. In a specific example in accordance with this embodiment, the antibody of the invention comprises an Fc domain having a substitution at position 234 with alanine and a substitution at position 235 with glutamic acid. In a yet more preferred embodiment, the antibody of the invention comprises an Fc having a substitution with alanine at position 234 and a substitution with alanine at position 235.

In other embodiments, the antibody of the invention comprises a Fc region, which variant Fc region exhibits reduced or abolished binding to one or more effector ligands as determined by standard assays known in the art and disclosed herein, relative to a comparable control molecule. In certain embodiments, the antibody of the invention has a Fc region that exhibits reduced or abolished binding to one or more effector ligands, which Fc region comprises a phenylalanine or proline at position 233; an alanine at position 234; an alanine or glutamic acid at position 235; an alanine at position 236, an alanine at position 237, an arginine at position 238; an alanine or glutamic acid at position 265; an alanine or asparagine at position 270; an alanine or glutamine at position 297; a phenylalanine, asparagine or proline at position 298; any amino acid at position 299 other than serine or threonine; or a combination of two or more of the above-listed substitutions. In certain embodiments, the antibody of the invention comprises an Fc domain having an alanine at position 265 and at position 297; an alanine at position 265 and a glutamine at position 297; a glutamic acid at position 265 and an alanine at position 297; or a glutamic acid at position 265 and a glutamine at position 297. In certain embodiments, the antibody of the invention comprises an Fc domain having an alanine at 234 and a glutamic acid at position 235. In preferred embodiments, the antibody of the invention comprises an Fc having an alanine at position 234 and an alanine at position 235.

Antibodies of the invention that comprise and Fc domain having an alanine at positions corresponding to 234 and 235 according to the numbering scheme of Kabat are known as “ala-ala” antibodies. In certain embodiments, use of “ala-ala” Fc domains and/or other combinations of amino acid combinations herein described (including combinations comprising “ala-ala” Fc domains) may abolish binding of the Fc domain to all FcγRs. The binding of a Fc domain to one or more FcγRs may be determined by any method described herein and/or known in the art.

In certain embodiments, the one or more amino acid modifications which abolish binding to all FcγRs or reduce or abrogate binding to one or more effector ligands comprise combinations of the modifications listed herein or combinations of the modifications listed herein with any that may confer null binding to any FcR (e.g., FcγRIIIA, FcγRIIIB, FcγRIIA) as determined by the methods disclosed herein or known to one skilled in the art. As readily understood by one of skill in the art, such antibodies of the invention may find particular use in the treatment of an autoimmune disease in that the anti-TCR antibodies serve to modulate immune function without the associated first-dose response common to anti-immune cell antibodies.

In certain embodiments, the anti-TCR antibodies of the invention, or antigen binding fragments thereof, have diminished (such as, but not limited to, less than 50%, less than 40%, less than 30%, less than 20%, less than 10%, less than 5% or less than 1% as compared to binding by a protein comprising a control Fc domain) or, more preferably, no detectable binding to one or more of any FcγR (e.g., FcγRI, FcγRII or FcγRIII) via its Fc domain as determined by assays routine in the art. In addition or alternatively, the anti-TCR antibodies of the invention, or antigen binding fragments thereof, may have diminished (such as, but not limited to, less than 50%, less than 40%, less than 30%, less than 20%, less than 10%, less than 5% or less than 1% as compared to binding by a control protein comprising a control Fc domain) or, more preferably, no detectable binding to any complement receptors, such as, C1q, as determined in routinely used assays. In particular embodiments, the antibody is aglycosylated. In other embodiments, the antibody lacks an Fc domain (e.g., is a Fab fragment, F(ab′)₂ or single chain antibody).

The antibodies of the invention are thus particularly useful because they have reduced or no in vivo toxicity caused by lymphokine production or cytokine release. Methods of measuring lymphokine production and cytokine release are known and routine in the art and encompassed herein. For example, cytokine release may be measured by measuring secretion of cytokines including but not limited to Interleukin-2 (IL-2). Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-β, TFN-γ, IGF-I, IGF-II. For example, see, Isaacs et al., 2001, Rheumatology, 40: 724-738; Soubrane et al., 1993, Blood, 81(1): 15-19; each of which is incorporated herein by reference in its entirety.

The invention relates to antibodies, or antigen binding fragments thereof, that specifically bind the TCR, preferably the extracellular domain of TCR, and most preferably the extracellular constant domain of the α chain of TCR. Assays to determine the binding specificity of an antibody or antigen binding fragment thereof, for an antigen or epitope include, but are not limited to ELISA, western blot, surface plasmon resonance (e.g., BIAcore) and radioimmunoassay. Any method known in the art for assessing protein-protein binding specificity may be used to identify antibodies or antigen binding fragments of the invention that exhibit a Kd of greater than 0.001 nM but not greater than 5 nM, not greater than 10 nM, not greater than 15 nM, not greater than 20 nM, not greater than 25 nM, not greater than 30 nM, not greater than 35 nM, not greater than 40 nM, not greater than 45 nM, or not greater than 50 nM. In certain embodiments, the isolated V_{H} or V_{L} domains of the invention exhibit a Kd of no greater than 5 nM, no greater than 10 nM, no greater than 15 nM, no greater than 20 nM, no greater than 25 nM, no greater than 30 nM, no greater than 35 nM, no greater than 40 nM, no greater than 45 nM, or no greater than 50 nM, for example, as determined by BIAcore assay.

The present invention also provides for antibodies, or antigen binding fragments thereof, that exhibit high binding affinity for the TCR preferably the extracellular domain of TCR, and most preferably the extracellular domain of the α chain of TCR. In a specific embodiment, an antibody of the present invention or antigen binding fragment thereof has an association rate constant or kₒₙ rate (antibody (Ab)+antigen (Ag) Ab−Ag) of at least 10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M⁻¹ s⁻¹, at least 5×10⁷ M⁻¹ s⁻¹, or at least 10⁸ M⁻¹ s⁻¹. In a preferred embodiment, the antibody of the present invention or antigen-binding fragment thereof has a kₒₙ of at least 2×10⁵ M⁻¹ s⁻¹, at least 5×10⁵ M⁻¹ s⁻¹, at least 10⁶ M⁻¹ s⁻¹, at least 5×10⁶ M⁻¹ s⁻¹, at least 10⁷ M M⁻¹ s⁻¹, at least 5×10⁷ M⁻¹ s⁻¹, or at least 10⁸ M⁻¹ s⁻¹.

In another embodiment, the antibody of the present invention or antigen-binding fragment thereof has a k_{off} rate (antibody (Ab)+antigen (Ag) Ab−Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹. In a preferred embodiment, an antibody of the present invention or antigen-binding fragment thereof has a k_{off} of less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻⁹ s⁻¹.

In still other embodiments, the antibody of the present invention or antigen-binding fragment thereof has an affinity constant or Kₐ. (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an antibody of the present invention or antigen-binding fragment thereof has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴M, less than 5×10⁻¹⁴ M, less than 10⁻¹⁵ M, or less than 5×10⁻¹⁵ M.

In certain embodiments of the invention, the anti-TCR antibodies are monoclonal antibodies, synthetic antibodies, recombinantly produced antibodies, multispecific antibodies, human antibodies, chimeric antibodies, camelized antibodies, single-chain Fvs (scFv), single chain antibodies, Fab fragments, F(ab′) fragments, disulfide-linked Fvs (sdFv), intrabodies, or epitope-binding fragments of any of the above. In certain embodiments, the antibodies of the invention, or antigen fragments thereof, are covalent diabodies such as those disclosed in U.S. patent application publication US 2007/0004909 by Johnson et al. or are Ig-DARTS such as those disclosed in U.S. patent application Ser. Nos. 61/019,051 and 12/138,867, each by Johnson et al. (each of which is hereby incorporated by reference in its entirety).

The antibodies of the invention, or antigen binding fragments thereof, may be humanized by any method known in the art for modifying proteins for therapeutic use in humans. In addition to methods commonly known in the art for combining heterologous CDR sequences with human framework and/or constant domains, the term “humanization” also includes methods of protein and/or antibody resurfacing such as those disclosed in U.S. Pat. Nos. 5,770,196; 5,776,866; 5, 821,123; and 5,896,619, each to Studnicka et al. (each of which is incorporated herein by reference in its entirety).

The antibodies of the invention, or antigen binding fragments thereof may derived from any species (e.g., rabbit, mouse, rat, donkey, cow, sheep, goat, horse, primate), but are preferably derived from human immunoglobulin molecules that can be of any type (e.g., IgG, IgE, IgM, IgD, IgA and IgY), or class (e.g., IgG₁, IgG₂, IgG₃, IgG₄, IgA₁, and IgA₂) or subclass. The antibodies of the invention, or antigen binding fragments thereof, can be produced by any method known in the art, for example, chemical synthesis or recombinant techniques.

The present invention provides methods of treating, preventing, slowing the progression of and/or ameliorating the symptoms of T cell mediated immunological diseases, particularly autoimmune diseases and T-cell cancers, in subjects diagnosed with such diseases (and/or in subjects predisposed to developing such diseases or disorders), by administering to a subject in need thereof a therapeutically or prophylactically effective amount of an TCR antibody, or antigen binding fragment thereof. In certain embodiments, the invention provides an antibody for the treatment of an autoimmune disease, wherein said antibody comprise a variant Fc region that exhibits decreased binding to one or more Fc effector ligands relative to that of a wild-type Fc region.

In certain embodiments, the antibodies of the invention are useful for prevention or treatment of T-cell mediated diseases, in particular, T-cell cancers and/or malignancies, as a single agent therapy. In certain embodiments, the invention provides an antibody for the treatment of a T-cell cancer or malignancy, wherein said antibody comprise a variant Fc region that exhibits increased binding to one or more Fc effector ligands relative to that of a wild-type Fc region. The present invention provides methods for preventing, treating, managing or ameliorating a T-cell malignancy, or one or more symptoms thereof, said methods comprising administering to a subject in need thereof a prophylactically or therapeutically effective amount of a TCR antibody of the invention, or an analog, derivative, variant or an antigen-binding fragment thereof. In particular, the invention provides methods for preventing, treating, managing, or ameliorating indolent or aggressive T-cell leukemias, T-cell lymphomas, acute lymphoblastic leukemia, adult T-cell leukemia or Hodgkin's lymphoma. Additional T-cell lymphoproliferative disorders that may be treated according to the methods of the invention include, but are not limited to, thymic and post-thymic malignancies; tumors of lymphoid progenitor cells, thymic cells, stromal cells, epithelial cells, thymocytes, T-cells, natural killer (“NK”) cells, or antigen-presenting cells; lymphomas; thymomas; and non-Hodgkin's disease; lymphoblastic lymphoma; anaplastic large cell lymphoma; peripheral T-cell lymphomas; angioimmunoblastic lymphoma; angiocentric lymphoma (nasal T-cell lymphoma); and intestinal T-cell lymphoma. In particular embodiments, the cancer of the subject is refractory to one or more standard or experimental therapies. In specific embodiments, the antibodies of the invention for use in the prevention or treatment of T-cell malignancies exhibit binding to one or more effector ligands or exhibit one or more effector-ligand binding-mediated activities comparable to that of a control antibody comprising a wild-type or non-modified Fc domain (e.g., a control antibody comprising a Fc domain that does not comprise one or more amino acid modifications described herein and/or known in the art to reduce or abrogate binding of the Fc domain to one or more effector ligands). In certain embodiments, the antibody of the invention for use in the prevention or treatment of T-cell malignancies comprises a wild-type Fc domain, or comprises a Fc domain that does not comprise one or more amino acid modifications described herein and/or known in the art to reduce or abrogate binding of the Fc domain to one or more FcRs.

The invention provides for the use of a TCR-specific antibody in combination with a standard or experimental treatment regimen for T-cell malignancies (e.g., chemotherapy, radioimmunotherapy, or radiotherapy). Such combination therapy may enhance the efficacy of standard or experimental treatment. Examples of therapeutic agents that are particularly useful in combination with a TCR-specific antibody or an antigen-binding fragment thereof, for the prevention, treatment, management, or amelioration of T-cell malignancies, include, but are not limited to, CAMPATH™, anti-Tac, purine analogs, pentostatin, cytotoxic agents, anti-retroviral agents, arsenic trioxide, interferon-alpha, and/or chemotherapeutic and anti-cancer agents known in the art. Chemotherapeutic agents include, but are not limited to alkylating agents, antimetabolites, natural products, and hormones. The combination therapies of the invention may enable lower dosages of an anti-TCR antibody or an antigen-binding fragment thereof and/or less frequent administration of anti-TCR antibody or an antigen-binding fragment thereof to a subject with a T-cell malignancy, to achieve a therapeutic or prophylactic effect.

In another embodiment, the use of an anti-TCR antibody or an antigen-binding fragment thereof prolongs the survival of a subject diagnosed with a T-cell malignancy or prevents the worsening of one or more symptom associated with the T-cell malignancy in a subject diagnosed with the malignancy.

In certain embodiments, diagnosis of T-cell malignancy is based on the detection of an abnormal concentration of T-lymphocytes in the peripheral blood of the subject and/or target tissue of the malignancy. In certain embodiments, the anti-TCR antibody of the invention is administered to achieve a reduction by at least 10%, at least 20%, at least 35%, at least 30%, at least 40%, at least 45%, at least 50%, at least 55%, at least 60%, at least 65%, at least 70%, at least 75%, at least 80%, or at least 85% in absolute number, or concentration, of the subject's T-lymphocytes as determined by immunospot assay (e.g., ELISPOT) relative to the pretreatment condition, said determinations made at 6 months, 9 months, 12 months, 15 months, 18 months, or 24 months after the previous treatment.

In certain embodiments, the invention provides for the use of a TCR-specific antibody, or antigen binding fragment thereof conjugated to a therapeutic agent or drug. Examples of therapeutic agents which may be conjugated to an anti-TCR antibody or an antigen-binding fragment thereof include, but are not limited to, cytokines, toxins, radioactive elements, and antimetabolites.

The invention also provides methods of treating, preventing, slowing the progression of and/or ameliorating the symptoms of T-cell mediated diseases or disorders, including graft rejection, graft versus host disease, unwanted delayed-type hypersensitivity reactions (such as delayed-type allergic reactions), T-cell mediated pulmonary diseases, and autoimmune diseases. T-cell mediated pulmonary diseases include sarcoidosis, hypersensitivity pneumonitis, acute interstitial pneumonitis, alveolitis, pulmonary fibrosis, idiopathic pulmonary fibrosis and other diseases characterized by inflammatory lung damage. T-cell autoimmune diseases include multiple sclerosis, neuritis, polymyositis, psoriasis, vitiligo, Sjogren's syndrome, rheumatoid arthritis, Type 1 diabetes, autoimmune pancreatitis, inflammatory bowel diseases (e.g., Crohn's disease and ulcerative colitis), celiac disease, glomerulonephritis, scleroderma, sarcoidosis, autoimmune thyroid diseases (e.g., Hashimoto's thyroiditis and Graves disease), myasthenia gravis, Addison's disease, autoimmune uveoretinitis, pemphigus vulgaris, primary biliary cirrhosis, pernicious anemia, and systemic lupus erythematosis, lupus (particularly, cutaneous), effects from organ transplantation, graft vs. host disease (GVHD), etc. Particularly, the methods of the invention are advantageous in subjects with early stage disease to slow or reduce the damage from the autoimmunity and maintain a high level of function and/or reduce the need for other therapy (e.g., in the treatment or prophylaxis of Type I diabetes, the methods of the invention may reduce the need for exogenous insulin administration in the subject). In addition, the methods of the invention may advantageously reduce the incidence of or result in no incidence of cytokine release syndrome previously associated with administration of therapeutic antibodies, and, in particular, anti-T-cell (e.g., anti-TCR) antibodies.

In certain embodiments, the antibody of the invention for use in the prevention or treatment of T-cell mediated diseases or disorders, which diseases or disorders are not T-cell malignancies (e.g., are autoimmune diseases or disorders), comprises a Fc domain that exhibits reduced or abrogated binding to one or more effector ligands. In specific embodiments, the antibody of the invention for use in the prevention or treatment of T-cell mediated diseases or disorders, which diseases or disorders are not T-cell malignancies (e.g., are autoimmune diseases or disorders), does not comprise an Fc domain. In other embodiments, the antibody of the invention for use in the prevention or treatment of T-cell mediated diseases or disorders, which diseases or disorders are not T-cell malignancies (e.g., are autoimmune diseases or disorders), comprises an Fc domain that has been modified relative to a control antibody to reduce or abrogate binding of the Fc domain to one or more effector ligands. Such modifications include, but are not limited to, amino acid modifications (including substitution, insertion or deletion) that modify the glycosylation pattern of the Fc domain relative to a control Fc domain and amino acid modifications that reduce or abrogate the binding of the Fc domain to one or more FcRs. Such modifications may be selected from any modification described herein and/or known in the art to reduce or abrogate binding of a polypeptide to one or more effector ligands.

In other embodiments, the invention provides methods of preventing or delaying the onset of an autoimmune disease or disorder in a subject predisposed to developing said disease or disorder, but which subject has yet to experience symptoms of or be diagnosed with said disease or disorder according to criteria accepted in the art (e.g., in Type I diabetes, a diagnosis according to criteria established by the American Diabetes Association: see, e.g., Mayfield et al., et al., 2006, Am. Fam. Physician 58:1355-1362, hereby incorporated by reference herein in its entirety). In other embodiments, administration of an antibody of the invention prevents onset and/or development of the disorder, prevents onset of symptoms of the disorder, and/or delays the positive diagnosis of said disorder by 2 months, 4 months, 6 months, 8, months, 10 months, 12 months, 15 months, 18 months, 21 months, or 24 months relative to a subject with similar clinical parameters who did not receive treatment.

The administration of the antibodies of the invention, or antigen biding fragments thereof, may delaying onset of the autoimmune disease or disorder, or, once diagnosable disease or disorder occurs, induce or reestablish self-tolerance, thereby slowing disease progression, or reducing and/or delaying the need to treat the symptoms of said disease. In addition, the invention provides methods of treatment such that a single round of treatment or round of treatment every 6 months, every 9 months, every 12 months, every 15 months, every 18 months, or every 24 months with an anti-TCR antibody (preferably, without any intervening treatment with anti-TCR antibodies), results in no or a minimal clinical progression of the disease or disorder as determined by standard methods 6 months, 9 months, 12 months, 15 months, 18 months, or 24 months after the previous round of treatment or the first round of treatment. In certain embodiments, a single round of treatment or round of treatment every 6 months, every 9 months, every 12 months, every 15 months, every 18 months, or every 24 months with an anti-TCR antibody (preferably, without any intervening treatment with anti-human TCR antibodies), decreases one or more of the clinical indices of the autoimmune disease or disorder in the patient by about 5%, about 10%, about 15%, about 20%, about 25%, about 30%, about 35%, about 40%, about 45%, about 50%, about 55%, about 60%, about 65% or about 70% as compared to pre-treatment levels at 6 months, 9 months, 12 months, 15 months, 18 months, or 24 months after the previous round of treatment or first round of treatment. In other embodiments, after treatment with a TCR antibody according to the invention in a single round of treatment or a round of treatment repeated every 6 months, every 9 months, every 12 months, every 15 months, every 18 months, or every 24 months (preferably, without any intervening treatment with anti-human TCR antibodies), one or more of the clinical indices of the autoimmune disease in the patient only increases by about 0.5%, about 1%, about 2.5%, about 5%, about 10%, about 15%, about 20%, about 25%, about 30%, about 35%, about 40%, about 45%, or about 50% as compared to pre-treatment levels at 6 months, 9 months, 12 months, 15 months, 18 months, or 24 months after the previous round of treatment or the first round of treatment. In other embodiments, after a single round of treatment or rounds of treatment every 6 months, every 9 months, every 12 months, every 15 months, every 18 months, or every 24 months with a TCR antibody according to the methods of the invention (preferably, without any intervening treatment with TCR antibodies), the average of one or more of the clinical indices of the autoimmune disease in the patient is only about 5%, about 10%, about 20%, about 30%, about 40%, about 50%, about 60%, about 70%, or about 80% that of the average of the same index in a patient that initiated conventional treatment with similar clinical parameters and was administered conventional treatment after the same amount of time, which levels were determined at 6 months, 9 months, 12 months, 15 months, 18 months, or 24 months after the previous round of treatment or the first round of treatment.

In certain embodiments, diagnosis of an autoimmune disorder or manifestation of a predisposition of an autoimmune disorder is based on the detection of cytotoxic T-lymphocytes (“CTLs”) that recognize donor specific antigens (i.e., autoreactive CTLs) in the peripheral blood of the subject and/or target tissue of the immune disorder. In certain embodiments, the anti-TCR antibody of the invention is administered to achieve a reduction by at least 10%, at least 20%, at least 35%, at least 30%, at least 40%, at least 45%, at least 50%, at least 55%, at least 60%, at least 65%, at least 70%, at least 75%, at least 80%, or at least 85% in absolute number, or proportion, of the subject's autoreactive CTLs as determined by immunospot assay (e.g., ELISPOT) relative to the pretreatment condition, said determinations made at 6 months, 9 months, 12 months, 15 months, 18 months, or 24 months after the previous treatment.

The invention also provides combination therapy methods. The methods of the invention can be carried out in combination with any standard treatment for the particular indication, such as standard immunosuppressant and/or anti-inflammatory treatments administered for the treatment or amelioration of autoimmune diseases. For example, with respect to the treatment of Type 1 diabetes, the TCR antibody therapy of the invention may be administered along with other therapies for diabetes, such as, but not limited to, administration of insulin, exenatide, pramlintide or a combination thereof. With respect to the treatment of multiple sclerosis, the TCR antibody therapy of the invention may be administered with other therapies known in the art for the treatment of multiple sclerosis, such as, but not limited to, administration of beta interferon (e.g., AVONEX®, BETASERON®, REBIF®), immunosuppressant (e.g., mitoxantrone), myelin basic protein copolymer 1 (e.g., COPAXONE®), or a combination thereof. The TCR antibodies of the invention may further be administered with other therapies such as anti IL-2 antibodies, cytokine antagonists, and steroidal therapies (for example, but not limited to, glucocorticoids, dexamethasone, cortisone, hydrocortisone, prednisone, prednisolone, triamcinolone, azulfidine, etc.), non-steroidal anti-inflammatories (NSAIDS), such as, but not limited to aspirin, ibuprofen, diclofenac, etodolac, fenoprofen, indomethacin, ketolorac, oxaprozin, nabumetone, sulindac, tolmentin, naproxen, or ketoprofen, immunosuppressants, such as, methotrexate or cyclosporin, and TNF-α inhibitors such as, but not limited to, etanercept and infliximab. In certain embodiments of the invention, subjects which have become refractory to conventional treatments are treated using methods of the invention. In certain embodiments, the anti-TCR antibody is administered in combination with one or more antigens that characterize the autoimmune disease or disorder (i.e., antigens which are bound by autoantibodies (i.e., self-reactive antibodies) found in patients with said autoimmune disease or disorder). For example, in the treatment of type 1 diabetes, the antibodies of the invention may be administered in combination with islet cell antigens, such as GAD and IA-2; in the treatment of multiple sclerosis, the antibodies of the invention may be administered in combination with one or more of the putative autoantigens αB-crystailin, S 100β, proteolipid protein (PLP) and/or myelin oligodendrocyte glycoprotein (MOG).

The present invention encompasses antibodies recombinantly fused or chemically conjugated (including both covalently and non-covalently conjugations) to therapeutic agents, e.g., heterologous polypeptides (i.e., an unrelated polypeptide; or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences or through chemical conjugation. The antibodies of the invention may be used for example to target the therapeutic agent to particular cell types (e.g., T-cells), either in vitro or in vivo, by fusing or conjugating the agent to the antibody or antigen binding fragment of the present invention that is specific for the particular cell surface receptor (i.e., TCR). The therapeutic-antibodies or antigen binding fragments thereof may also be used in in vitro immunoassays and purification methods using methods known in the art. See e.g., PCT publication Number WO 93/21232; EP 439,095; Naramura et al., Immunol. Lett., 39:91-99, 1994; U.S. Pat. No. 5,474,981; Gillies et al., PNAS, 89:1428-1432, 1992; and Fell et al., J. Immunol., 146:2446-2452, 1991, which are incorporated herein by reference in their entireties. Therapeutic agents for use in accordance with the methods of the invention are not to be construed as limited to classical chemical therapeutic agents (e.g., chemotherapeutics), but may include peptides that may or may not modify or alter biological responses, drug moieties, radioactive materials, macrocyclic chelators, and siRNA. In non-limiting examples, polypeptides may include albumin, protamine, albumin interacting proteins (e.g., gp60, gp30, gp18), protein A, a G protein, protein transduction domains (see e.g., Bogoyevitch et al., 2002, DNA Cell Biol 12:879-894, hereby incorporated by reference in its entirety), toxins, cytotoxins, or portions of antibody molecules (e.g., Fc domain, CH₁ domain, CH₂ domain, CH₃ domain, hinge domain, CL domain, etc.); such radionuclides may include radionuclides (e.g., alpha-, beta-, gamma-emitters, etc.) known in the art for labeling (i.e., producing a detectable signal in vivo or in vitro) and/or producing a therapeutic effect (e.g., ¹²⁵I, ¹³¹I, ¹⁴C, etc.); such macrocyclic chelators include those known in the art for conjugating radiometal ions (e.g., DOTA).

In preferred embodiments, the patient is under 21 years of age, 18 years of age, under 15 years of age, under 12 years of age, under 9 years of age, or under 5 years of age or from infancy to 3 years of age, from 2 to 5 years of age, from 5 to 9 years of age, from 9 to 12 years of age, from 12 to 20 years of age. In other embodiments, the patient is an adult.

The TCR antibodies of the invention may be administered as a single course therapy, e.g., by bolus injection or over a longer period of time by, e.g., intravenous administration. The antibodies of the invention may be administered as a bolus or as a series of boluses. Such boluses may be delivered over a staggered time course with, e.g., 1, 2, 3, 4, 5, 6, 7, 8, 9, 10, 11, 12, 13, 14, 15, 16, 17, 18, 19, 20, 22, 24, 26, 28, 30, or more days between successive bolus administrations. In specific embodiments, the TCR antibodies of the invention are administered as part of a treatment regimen wherein multiple doses of the anti-TCR antibodies are administered over multiple days for a total of, e.g., 2, 3, 4, 5, 6, 7, 8, 9, 10, 11, 12, 13, or 14 doses per regimen. The multiple administrations need not be on consecutive days, but can be separated by 1 day, 2 days, 3 days, 4 days, 5 days, 6 days, 1 week, 1.5 weeks or 2 weeks. In certain embodiments, the dose on the first day of the regimen is ½, ¼, ⅕, ⅛, 1/10, 1/12, 1/15 or 1/20 that of the therapeutic dose and the dosages administered on subsequent days of the regimen escalate to the required daily dose by, e.g., the 3^{rd}, 4^{th}, 5^{th}, 6^{th} or 7^{th} administration of the regimen. In other embodiments, for the first 2, 3, 4, or 5 administrations of the anti-TCR antibody of the invention, the dose may increase by 1.5-fold, 2-fold, 3-fold, or 4-fold on each subsequent day. In specific embodiments, to reduce the possibility of cytokine release and other adverse effects, the first 1, 2, 3, or 4 doses or all the doses in the regimen are administered more slowly, relative to bolus injection, by intravenous administration. For example, the individual dose may be administered over about 5 minutes, about 15 minutes, about 30 minutes, about 45 minutes, about 1 hour, about 2 hours, about 4 hours, about 6 hours, about 8 hours, about 10 hours, about 12 hours, about 14 hours, about 16 hours, about 18 hours, about 20 hours, or about 22 hours. In certain embodiments, the dose is administered by slow infusion over a period of, e.g., 20 to 24 hours. In specific embodiments, the dose is infused by a pump, preferably increasing the concentration of antibody administered as the infusion progresses. Alternatively, the total daily dose may be divided into two or more equal portions and administered as bolus infusions over the day at intervals of 6, 8, 10 or 12 hours to reduce the level of cytokine release caused by administration of the antibody. In other embodiments, a set fraction of the total daily dose is administered in escalating doses. In other embodiments, doses in the regimen are administered for a certain number of consecutive days, followed by a certain number of days without any doses administered, followed again by doses administered on a certain number of consecutive days and so on until, for example, 14 (or, e.g., 6, 7, 8, 9, 10, 11, 12, 13, 15, 16, 17, 18, 19 or 20) doses are administered in total. For example, the day 1, day 2, day 3 and day 4 doses of one of the regimens may be administered over four consecutive days and then three days without any doses and then the day 5, 6, 7 and 8 doses are administered, followed by another three days without doses, and then the day 9, 10, 11, 12 day doses, with three days off, and finally the day 13 and 14 doses.

In certain embodiments, the antibodies of the invention are administered to the subject in need thereof using a mode other than intravenous administration that provides the pharmacological equivalent to total daily dose of anti-TCR antibody as administered intravenously. The invention further contemplates methods in which the patient is chronically administered low doses of the anti-TCR antibody and methods in which the patient is administered one or more additional rounds of the anti-TCR antibody treatment regimen approximately 6 months, 9 months, 12 months, 18 months, 2 years, 3 years or 5 years after the initial treatment, optionally depending on clinical parameters, or is administered another round of treatment with anti-TCR antibody every approximately 6 months, 9 months, 12 months, 18 months, 2 years, 3 years or 5 years, optionally depending on clinical parameters.

The antibodies of the invention are administered in an amount effective to modulate the immune system. Those of skill in the art will be able to employ known methods of determining appropriate dosages and the teachings of this specification to determine appropriate dosage time-courses and amounts. In certain embodiments, the antibodies of the invention are administered at a dosage ranging from 1 pg/kg to 20,000 pg/kg, from 10 pg/kg to 2,000 pg/kg or from 10 pg/kg to 1,000 pg/kg.

In certain embodiments, the anti-human TCR antibody is administered so as to achieve a certain level of combined coating and modulation of T cell receptor complexes on T cells, as determined by methods well known in the art, see, e.g., Example 11 of U.S. patent application publication US 2003/0108548, which is hereby incorporated by reference in its entirety. In specific embodiments, the dosing regimen achieves a combined T cell receptor coating and modulation of at least 50%, 60%, 70%, 80%, 90%, 95% or of 100% with, in specific embodiments, little to no detectable free anti-human TCR antibody (for example, less than 200 ng/mL the drug detected in the blood of the patient by standard methods known in the art).

In specific embodiments, the anti-human TCR antibody is not administered by daily doses over a number of days, but is rather administered by infusion in an uninterrupted manner over 4 hours, 6 hours, 8 hours, 10 hours, 12 hours, 15 hours, 18 hours, 20 hours, 24 hours, 30 hours or 36 hours. The infusion may be constant or may start out at a lower dosage for, for example, the first 1, 2, 3, 5, 6, or 8 hours of the infusion and then increase to a higher dosage thereafter. Over the course of the infusion, the patient receives a dose equal to the amount administered, for example, in a multiple dose regimen described above. In particular, the speed and duration of the infusion is designed to minimize the level of free anti-TCR antibody in the subject after administration. In certain embodiments, the level of free anti-TCR antibody should not exceed 200 ng/ml free antibody. In addition, the infusion is designed to achieve a combined T cell receptor coating and modulation of at least 50%, 60%, 70%, 80%, 90%, 95% or of 100%.

In other embodiments, the anti-TCR antibody is administered chronically to treat, manage, maintain, prevent, or slow the progression of or delay the onset of the autoimmune disease or disorder. For example, in certain embodiments, a low dose of the anti-TCR antibody is administered once a month, twice a month, three times per month, once a week or even more frequently either as an alternative to the 6 to 14 day dosage regimen discussed above or after administration of such a regimen to enhance or maintain its therapeutic effect. Such a low dose may be anywhere from 1 μg/m² to 100 μg/m², preferably, approximately 5 μg/m², 10 μg/m², 15 μg/m², 20 μg/m², 25 μg/m², 30 μg/m², 35 μg/m², 40 μg/m², 45 μg/m², or 50 μg/m². In certain embodiments, the anti-TCR antibody is administered chronically subsequent to administration of a dosing regimen as described above, for example, to maintain the therapeutic effect of the regimen.

In other embodiments, the subject may be re-dosed at some time subsequent to administration of the anti-TCR antibody dosing regimen, preferably, based upon one or more physiological parameters, but may be done as a matter of course. Such redosing may be administered and/or the need for such redosing evaluated 6 months, 9 months, 1 year, 15 months, 18 months, 2 years, 30 months or 3 years after administration of a dosing regimen and may include administering a course of treatment every 6 months, 9 months, 1 year, 15 months, 18 months, 2 years, 30 months or 3 years.

In specific embodiments, subjects are administered a subsequent round of anti-TCR antibody treatment based upon one or a combination of the CD4/CD8 cell ratio, CD8 cell count, CD4/CD3 inversion, CD4/CD25 cell ratio, CD4/FoxP3 cell ratio, CD4/CD40 cell ratio, CD4/IL-10 cell ratio, and/or CD4/TGF-β cell ratio as is well known in the art. Other parameters for determining whether to administer a subsequent round of treatment include an appearance or worsening of diagnostic indicators for the autoimmune disease or disorder as described herein and/or known in the art.

In preferred embodiments, the anti-TCR antibodies are administered parenterally, for example, intravenously, intramuscularly or subcutaneously, or, alternatively, are administered orally. The anti-human TCR antibodies may also be administered as a sustained release formulation.

The invention also encompass polynucleotides that encode the antibodies of the invention. In one embodiment, the invention provides an isolated nucleic acid sequence encoding a heavy chain or a light chain of an antibody or a fragment thereof that specifically binds the TCR, preferably the extracellular domain of the TCR, and most preferably, the extracellular domain of the α chain of TCR. The invention also relates to a vector comprising said nucleic acid. The invention further provides a vector comprising a first nucleic acid molecule encoding a heavy chain and a second nucleic acid molecule encoding a light chain, said heavy chain and light chain being of an antibody or a fragment thereof that specifically binds TCR, preferably the extracellular domain of the TCR, and most preferably, the extracellular domain of the α chain of TCR. In one specific embodiment, said vector is an expression vector. The invention further provides host cells containing the vectors of or polynucleotides encoding the antibodies of the invention.

The present invention encompasses methods for the production of antibodies of the invention or fragments thereof, particularly for the production of novel monoclonal antibodies (“MAb”) with specificity for TCR, preferably the extracellular domain of the TCR, and most preferably, the extracellular constant domain of the α chain of TCR. The antibodies of the invention or fragments thereof can be produced by any method known in the art for the production of antibodies, in particular, by secretion from cultured hybridoma cells, chemical synthesis or by recombinant expression techniques known in the art. In one specific embodiment, the invention relates to a method for recombinantly producing a TCR-specific antibody, said method comprising: (i) culturing under conditions suitable for the expression of said antibody in a medium, a host cell containing a first nucleic acid molecule, operably linked to a heterologous promoter and a second nucleic acid operably linked to the same or a different heterologous promoter, said first nucleic acid and second nucleic acid encoding a heavy chain and a light chain, respectively, of an antibody or a fragment thereof that specifically binds TCR, preferably the extracellular domain of the TCR, and most preferably, the extracellular domain of the α chain of TCR; and (ii) recovery of said antibody from said medium.

3.1 Terminology

As used herein, the term “about” or “approximately,” when used in conjunction with a number, refers to any number within 1, 5 or 10% of the referenced number or within experimental error typical of methods used for measurement.

As used herein, the term “analog” in the context of polypeptides refers to a polypeptide that possesses a similar or identical function as a second polypeptide but does not necessarily comprise a similar or identical amino acid sequence of the second polypeptide, or possess a similar or identical structure of the second polypeptide. A polypeptide that has a similar amino acid sequence refers to a second polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second polypeptide; (b) a polypeptide encoded by a nucleotide sequence that hybridizes under stringent conditions to a nucleotide sequence encoding a second polypeptide of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second polypeptide. A polypeptide with similar structure to a second polypeptide refers to a polypeptide that has a similar secondary, tertiary or quaternary structure to the second polypeptide. The structure of a polypeptide can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possess a similar or identical function as a first organic or inorganic molecule and is structurally similar to the first organic or inorganic molecule.

As used herein, the terms “antagonist” and “antagonists” refer to any protein, polypeptide, peptide, antibody, antibody fragment, large molecule, or small molecule (less than 10 kD) that blocks, inhibits, reduces or neutralizes the function, activity and/or expression of another molecule. In various embodiments, an antagonist reduces the function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS) and as determined by any method known in the art and/or described herein.

As used herein, the terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antibodies of the invention), and epitope-binding fragments of any of the above. The terms “antibody” and “antibodies” also refer to covalent diabodies such as those disclosed in U.S. patent application publication US 2007/0004909 by Johnson et al. and Ig-DARTS such as those disclosed in U.S. patent application Ser. Nos. 61/019,051 and 12/138,867 each by Johnson et al. (each of which is hereby incorporated by reference in its entirety). In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

As used herein, the term “derivative” in the context of polypeptides refers to a polypeptide that comprises an amino acid sequence which has been altered by the introduction of amino acid residue substitutions, deletions or additions. The term “derivative” as used herein also refers to a polypeptide that has been modified, i.e, by the covalent attachment of any type of molecule to the polypeptide. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative polypeptide may be produced by chemical modifications using techniques known to those of skill in the art, including, but not limited to specific chemical cleavage, acetylation, formylation, metabolic synthesis of tunicamycin, etc. Further, a derivative polypeptide may contain one or more non-classical amino acids. A polypeptide derivative possesses a similar or identical function as the polypeptide from which it was derived.

As used herein, the terms “disorder” and “disease” are used interchangeably to refer to a condition in a subject. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs.

As used herein, the term “epitopes” refers to fragments of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

As used herein, the term “Fc region” is used to define a C-terminal region of an IgG heavy chain. Although the boundaries may vary slightly, the human IgG heavy chain Fc region is defined to stretch from Cys226 to the carboxy terminus. The Fc region of an IgG comprises two constant domains, CH2 and CH3. The CH2 domain of a human IgG Fc region usually extends from amino acids 231 to amino acid 341. The CH3 domain of a human IgG Fc region usually extends from amino acids 342 to 447. The Fc region of an IgG comprises two constant domains, CH2 and CH3. The CH2 domain of a human IgG Fc region (also referred to as “Cγ2” domain) usually extends from amino acid 231-340. The CH2 domain is unique in that it is not closely paired with another domain. Rather, two N-linked branched carbohydrate chains are interposed between the two CH2 domains of an intact native IgG.

Throughout the present specification, the numbering of the residues in an IgG heavy chain corresponds to that of the EU index as in Kabat et al., Sequences of Proteins of Immunological Interest, 5^{th} Ed. Public Health Service, NH1, MD (1991), expressly incorporated herein by references. The “EU index as in Kabat” refers to the numbering of the human IgG1 EU antibody. The residues of the IgG heavy chain of the invention and that of the EU index as in Kabat are aligned according to methods known in the art to identify corresponding residues.

The “hinge region” is generally defined as stretching from Glu216 to Pro230 of human IgG1. Hinge regions of other IgG isotypes may be aligned with the IgG1 sequence by placing the first and last cysteine residues forming inter-heavy chain S—S binds in the same positions.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a second polypeptide. In a specific embodiment, the fragment is a functional fragment in that it retains at least one function of the second polypeptide.

As used herein, the term “fusion protein” refers to a polypeptide that comprises an amino acid sequence of a first protein or functional fragment, analog or derivative thereof, and an amino acid sequence of a heterologous protein (i.e., a second protein or functional fragment, analog or derivative thereof different than the first protein or functional fragment, analog or derivative thereof). In particular embodiments, a fusion protein comprises a TCR binding molecule and a heterologous protein, polypeptide, or peptide.

As used herein, the term “host cell” refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “hybridizes under stringent conditions” describes conditions for hybridization and washing under which nucleotide sequences at least 60% (65%, 70%, preferably 75%, 80% or 85%, and more preferably, 90% or 95%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “immunomodulatory agent” and variations thereof refer to an agent that modulates a host's immune system. In certain embodiments, an immunomodulatory agent is an immunosuppressant agent. In certain other embodiments, an immunomodulatory agent is an immunostimulatory agent. Immunomodulatory agents include, but are not limited to, small molecules, peptides, polypeptides, fusion proteins, antibodies, inorganic molecules, mimetic agents, and organic molecules.

As used herein, the term “immunospecifically binds to an antigen” and analogous terms refer to peptides, polypeptides, fusion proteins and antibodies or fragments thereof that specifically bind to an antigen or a fragment and do not specifically bind to other antigens. A peptide or polypeptide that immunospecifically binds to an antigen may bind to other peptides or polypeptides with lower affinity as determined by, e.g., immunoassays, BIAcore, or other assays known in the art. Antibodies or fragments that immunospecifically bind to an antigen may cross-reactive with related antigens. Preferably, antibodies or fragments that immunospecifically bind to an antigen do not cross-react with other antigens.

As used herein, the term “immunospecifically binds to TCR” and analogous terms refer to peptides, polypeptides, fusion proteins and antibodies or fragments thereof that specifically bind to one or more polypeptides, or fragments thereof, that form the TCR complex and do not specifically bind to other polypeptides. In preferred embodiments the anti-TCR antibodies of the invention do not detectably bind CD3. A peptide or polypeptide that immunospecifically binds to a TCR polypeptide may bind to other peptides or polypeptides with lower affinity as determined by, e.g., immunoassays, BIAcore, or other assays known in the art. Antibodies or fragments that immunospecifically bind to a TCR polypeptide may be cross-reactive with related antigens. Preferably, antibodies or fragments that immunospecifically bind to a TCR polypeptide, or fragment thereof, do not cross-react with other antigens. Antibodies or fragments that immunospecifically bind to a TCR polypeptide can be identified, for example, by immunoassays, BIAcore, or other techniques known to those of skill in the art. An antibody or fragment thereof binds specifically to a TCR polypeptide when it binds to a TCR polypeptide with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

As used herein, the term “in combination” refers to the use of more than one prophylactic and/or therapeutic agent. The use of the term “in combination” does not restrict the order in which prophylactic and/or therapeutic agents are administered to a subject with a disease or disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent (different from the first prophylactic or therapeutic agent) to a subject with a disease or disorder.

As used herein, the term “isolated” in the context of a peptide, polypeptide, fusion protein or antibody refers to a peptide, polypeptide, fusion protein or antibody which is substantially free of cellular material or contaminating proteins from the cell or tissue source from which it is derived, or substantially free of chemical precursors or other chemicals when chemically synthesized. The language “substantially free of cellular material” includes preparations of a peptide, polypeptide, fusion protein or antibody in which the peptide, polypeptide, fusion protein or antibody is separated from cellular components of the cells from which it is isolated or recombinantly produced. Thus, a peptide, polypeptide, fusion protein or antibody that is substantially free of cellular material includes preparations of a peptide, polypeptide, fusion protein or antibody having less than about 30%, 20%, 10%, or 5% (by dry weight) of heterologous protein (also referred to herein as a “contaminating protein”). When the peptide, polypeptide, fusion protein or antibody is recombinantly produced, it is also preferably substantially free of culture medium, i.e., culture medium represents less than about 20%, 10%, or 5% of the volume of the protein preparation. When the peptide, polypeptide, fusion protein or antibody is produced by chemical synthesis, it is preferably substantially free of chemical precursors or other chemicals, i.e., it is separated from chemical precursors or other chemicals which are involved in the synthesis of the peptide, polypeptide, fusion protein or antibody. Accordingly such preparations of a peptide, polypeptide, fusion protein or antibody have less than about 30%, 20%, 10%, 5% (by dry weight) of chemical precursors or compounds other than the peptide, polypeptide, fusion protein or antibody of interest. In a preferred embodiment, a TCR binding molecule is isolated. In another preferred embodiment, an anti-human TCR antibody is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized and may be free of cDNA or other genomic DNA molecules, e.g., has been isolated from other clones in a nucleic acid library. In a preferred embodiment, a nucleic acid molecule encoding a TCR binding molecule is isolated. In another preferred embodiment, a nucleic acid molecule encoding an anti-human TCR antibody is isolated.

As used herein, the terms “non-responsive” and refractory” describe patients treated with a currently available prophylactic or therapeutic agent for an autoimmune disorder which is not clinically adequate to relieve one or more symptoms associated with the autoimmune disorder. Typically, such patients suffer from severe, persistently active disease and require additional therapy to ameliorate the symptoms associated with their autoimmune disorder.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the terms “prophylactic agent” and “prophylactic agents” refer to TCR binding molecules, e.g., antibodies or antigen binding fragments thereof, which can be used in the prevention, treatment, management or amelioration of one or more symptoms of an autoimmune disease. In preferred embodiments, the term “prophylactic agent” refers to anti-TCR antibodies (e.g., BMA 031 and variants and derivatives thereof).

As used herein, the term “prophylactically effective amount” refers to that amount of a TCR binding molecule sufficient to prevent the development, recurrence or onset of one or more symptoms of a disease or disorder.

As used herein, the terms “prevent”, “preventing” and “prevention” refer to the prevention of the recurrence or onset of one or more symptoms of a disease or disorder, e.g., autoimmune disorder or T cell malignancy in a subject resulting from the administration of a prophylactic or therapeutic agent.

As used herein, a “protocol” includes dosing schedules and dosing regimens. The protocols herein are methods of use and include prophylactic and therapeutic protocols. A “dosing regimen” or “course of treatment” may include administration of several doses of a therapeutic or prophylactic agent over 1 to 20 days.

As used herein, the phrase “side effects” encompasses unwanted and adverse effects of a prophylactic or therapeutic agent. Adverse effects are always unwanted, but unwanted effects are not necessarily adverse.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human.

As used herein, the term “synergistic” refers to a combination of prophylactic or therapeutic agents which is more effective than the additive effects of the agents in the combination when administered individually. A synergistic effect of a combination of prophylactic or therapeutic agents may permit the use of lower dosages of one or more of the agents and/or less frequent administration of said agents to a subject with an autoimmune disorder. The ability to utilize lower dosages of prophylactic or therapeutic agents and/or to administer said agents less frequently reduces the toxicity associated with the administration of said agents to a subjected without reducing the efficacy of said agents in the prevention or treatment of autoimmune disorders. In addition, a synergistic effect can result in improved efficacy of agents in the prevention or treatment of autoimmune disorders. Finally, synergistic effect of a combination of prophylactic or therapeutic agents may avoid or reduce adverse or unwanted side effects associated single agent therapy.

As used herein, the terms “therapeutic agent” and “therapeutic agents” refer to TCR binding molecules which can be used in the prevention, treatment, management or amelioration of one or more symptoms of a disease or disorder. In preferred embodiments, the term “therapeutic agent” refers to human or humanized anti-TCR antibodies (e.g., humanized BMA 031 and variants or derivatives thereof).

As used herein, the term “therapeutically effective amount” refers to that amount of a therapeutic agent sufficient to result in amelioration of one or more symptoms of a disorder. With respect to diabetes, a therapeutically effective amount preferably refers to the amount of therapeutic agent that reduces a subject's average daily insulin requirements by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%.

As used herein, the terms “treat”, “treatment” and “treating” refer to the amelioration of one or more symptoms associated with an autoimmune or inflammatory disorder that results from the administration of one or more TCR binding molecules. In particular, such terms refer to the amelioration of one or more symptoms associated with an autoimmune disorder or T cell malignancy that results from the administration of one or more anti-TCR antibodies

### 4. DESCRIPTION OF THE FIGURES

FIG. 1 Amino acid sequences of heavy chain variable domains from anti-TCR antibodies. SEQ ID NO:1, amino acid sequence of the heavy chain variable domain of murine BMA 031.

FIG. 2 Amino acid sequences of light chain variable domains from anti-TCR antibodies. SEQ ID NO:7, amino acid sequence of the light chain variable domain of murine BMA 031.

FIG. 3 Amino acid sequences of BMA 031 variable heavy and light chain CDRs.

FIG. 4 Average paw thickness in mice having collagen induced arthritis. Mice were treated with monoclonal antibodies H57 (hamster anti-murine TCR antibody), H57 AA (H57 with the “ala-ala” mutation) or 2C 11 (murine anti-CD3 antibody).

FIG. 5 Average paw thickness in the female mice from the treatment groups presented in FIG. 4.

FIG. 6 Average paw thickness in the male mice from the treatment groups presented in FIG. 4.

FIG. 7 Percentage of mice exhibiting paralysis score of at least 1 in a murine EAE model. Mice were treated with 50 μg of the murine anti-TCR antibody H57 AA on each of days 6-10 (n=7) or 10-14 (n=7) post EAE induction compared to no treatment (n=6).

FIG. 8 Survival of diabetic NOD mice after treatment with anti-TCR antibody. Mice were treated with 50 μg (n=8) or 100 μg (n=4) of the murine anti-TCR antibody H57 AA over the course of 5 days. The indicated days in FIG. 8 are post treatment. The survival of the treatment mice is compared to that of diabetic NOD controls treated with PBS (n=10).

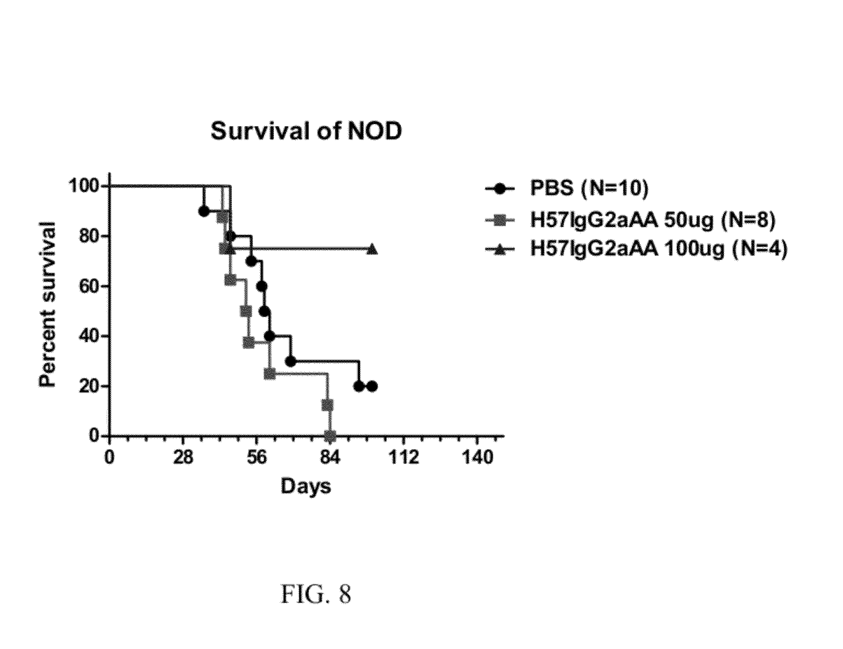

### 5. DETAILED DESCRIPTION OF THE INVENTION

The present invention presents antibodies that immunospecifically bind to the T cell receptor (TCR), preferably to the extracellular constant domain of the TCR α chain. In other embodiments, the antibody of the invention binds the β, γ or δ chain of TCR and, in specific embodiments, binds the constant region of the extracellular domain of the β, γ, or β chain of the TCR. The antibody of the invention may immunospecifically bind the α, β, γ or δ chain of the TCR as part of an intact TCR complex, or may bind the α, β, γ or δ chain as an individual peptide or fragment thereof.

In preferred embodiments, the invention provides an antibody, or antigen binding fragment thereof, that specifically binds the constant region of the α chain of the TCR or otherwise specifically binds the α chain regardless of clonal origin of the T-cell; in such embodiments, the antibody of the invention recognizes TCR α chains, and, thus the α/β TCR, generally (i.e., is a pan-specific TCR antibody, and, in particular, a pan-specific TCR α chain antibody). In other embodiments, the invention provides an antibody, or antigen binding fragment thereof, that specifically binds the constant region of the β chain of the TCR or otherwise specifically binds the β chain regardless of clonal origin of the T-cell; in such embodiments, the antibody of the invention recognizes TCR β chains, and, thus the α/β TCR, generally (i.e., is a pan-specific TCR antibody, and, in particular, a pan-specific TCR β chain antibody). In yet other embodiments, the invention provides an antibody, or antigen binding fragment thereof, that specifically binds the constant region of the γ chain of the TCR or otherwise specifically binds the γ chain regardless of clonal origin of the T-cell; in such embodiments, the antibody of the invention recognizes TCR γ chains, and, thus the γ/δ TCR, generally (i.e., is a pan-specific TCR antibody, and, in particular, a pan-specific TCR γ chain antibody). In still other embodiments, the invention provides an antibody, or antigen binding fragment thereof, that specifically binds the constant region of the δ chain of the TCR or otherwise specifically binds the δ chain regardless of clonal origin of the T-cell; in such embodiments, the antibody of the invention recognizes TCR δ chains, and, thus the γ/δ TCR, generally (i.e., is a pan-specific TCR antibody, and, in particular, a pan-specific TCR δ chain antibody).

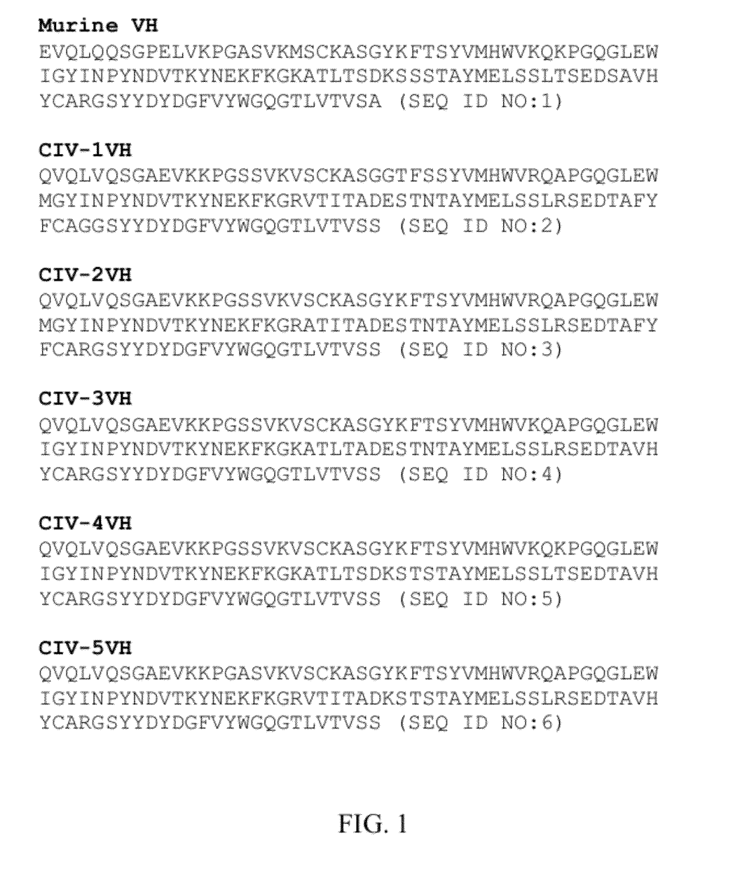

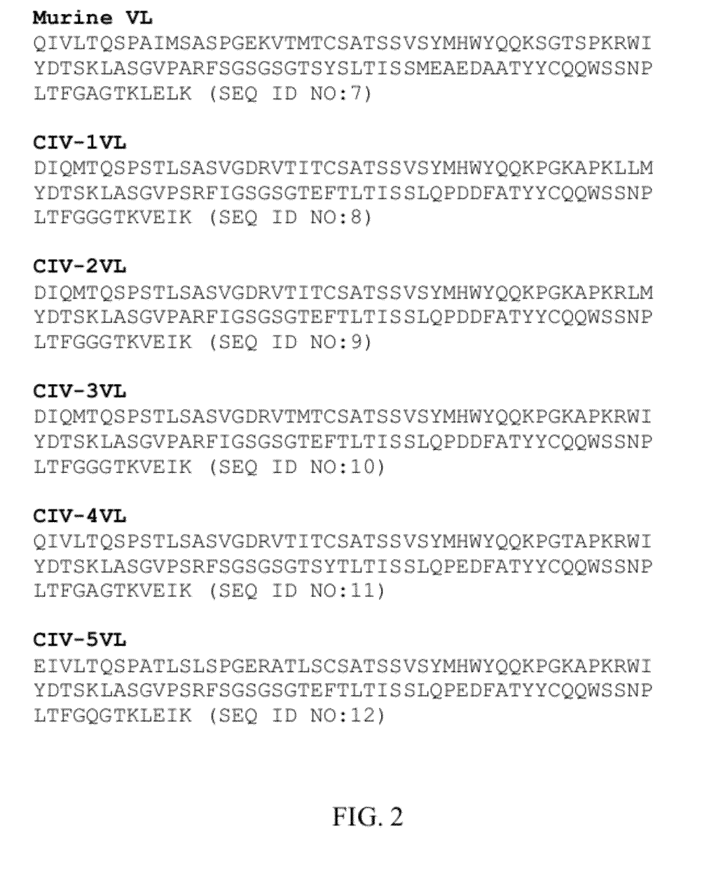

In a specific embodiment, the invention provides an antibody, or antigen binding fragment thereof, that is pan-specific for the TCR α chain. In a specific example in accordance with this embodiment, the antibody of the invention comprises a heavy or light chain variable domain derived from the heavy or light chain of the murine antibody BMA 031, as set forth in SEQ ID NO:1 (FIG. 1) and SEQ ID NO:7 (FIG. 2), respectively, and/or a derivative or variant thereof. In other embodiments, the antibody of the invention comprises one or more of the heavy and/or light chain CDRs from the murine antibody BMA 031, or a variant or derivative thereof. In other embodiments, the antibody of the invention, of an antigen binding fragment thereof, comprises a VH domain having the amino acid sequence of SEQ ID NO:2, SEQ ID NO:3, SEQ ID NO:4, SEQ ID NO:5 or SEQ ID NO:6 (as set forth in FIG. 1) and/or a VL domain having the amino acid sequence of SEQ ID NO:8, SEQ ID NO:9, SEQ ID NO:10, SEQ ID NO:11 or SEQ ID NO:12 (as set forth in FIG. 2), or a variant or derivative thereof. In other embodiments, the antibody of the invention, or an antigen binding fragment thereof, comprises one or more of a VH CDR1 having the amino acid sequence of SEQ ID NO:13 or SEQ ID NO:14, a VH CDR2 having the amino acid sequence of SEQ ID NO:15, a VH CDR3 having the amino acid sequence of SEQ ID NO:16, a VL CDR1 having the amino acid sequence of SEQ ID NO:17, a VL CDR2 having the amino acid sequence of SEQ ID NO:18, a VL CDR3 having the amino acid sequence of SEQ ID NO:19, and/or a variant or derivative thereof. The invention also encompasses TCR binding molecules comprising and amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and/or SEQ ID NO:12.

In alternate embodiments, the TCR binding protein for use according to the invention comprises a VL and/or VH domain and/or one or more CDRs with sequences derived from a non-human anti-TCR antibody, e.g., murine BMA 031, and one or more framework regions derived from framework sequences of one or more human immunoglobulins. A number of non-human anti-TCR monoclonal antibodies, from which CDRs and other sequences may be obtained, are known (e.g., 3H2906, IP26, T10B9, WT31, G-11, H-197, H-41, A-20, FL-189, H-20, H-250, C-17, H57, KJ1-26, KJ12-98, H28-710, UC3-10A6, GL3, 3H2928, 5K24, 3A10 and UC7-13D5 (see, e.g., Kubo et al., 1986, Mol Immunol 23:869-878; Itohara et al., 1989, PNAS USA 86:5094-5098; Kubo et al., 1989, J Immunol, 142:2736-2742; Waid et al., 1989, Am J Kidney Dis 14:61-70; Borst et al., 1990, Hum Immunol 29:175-188; Dent et al., 1990, Nature 343:714-719; Goodman et al., 1989, J Exp Med 170:1569-1581; Houlden et al., 1990, Cold Spring Harbor Symp Quant Biol 54:45 and Karaivanova et al., 1999, Hybridoma 18:497-503, each of which is hereby incorporated by reference in their entirety; H57 deposited with the ATCC® under ATCC® number HB-218™).

It is contemplated that, for some purposes, it may be advantageous to use TCR binding proteins that bind the TCR receptor, or a specific peptide thereof, at the same epitope bound by BMA 031, or at least sufficiently close to this epitope to block binding by BMA 031. Methods for epitope mapping and competitive binding experiments to identify binding proteins with the desired binding properties are well known to those skilled in the art of experimental immunology. See, for example, Harlow and Lane, cited supra; Stahl et al., 1983, Methods in Enzymology 9:242-53; Kirkland et al., 1986, J. Immunol. 137:3614-19; Morel et al., 1988, Molec. Immunol. 25:7-15; Cheung et al., 1990, Virology 176:546-52; and Moldenhauer et al., 1990, Scand. J. Immunol. 32:77-82. For instance, it is possible to determine if two antibodies bind to the same site by using one of the antibodies to capture the antigen on an ELISA plate and then measuring the ability of the second antibody to bind to the captured antigen. Epitope comparison can also be achieved by labeling a first antibody, directly or indirectly, with an enzyme, radionuclide or fluorophore, and measuring the ability of an unlabeled second antibody to inhibit the binding of the first antibody to the antigen on cells, in solution, or on a solid phase.

As explained in detail below, the TCR binding proteins (e.g., antibodies) of the invention may or may not comprise a human immunoglobulin Fc region. Fc regions are not present, for example, in scFv binding proteins. Fc regions are present, for example, in human or humanized tetrameric monoclonal IgG antibodies. In some embodiments of the present invention, the TCR binding protein includes an Fc region that has an altered effector function, e.g., reduced affinity for an effector ligand such as an Fc receptor or C1 component of complement compared to the unaltered Fc region (e.g., Fc of naturally occurring IgG₁ proteins). In one embodiment, the Fc region is not glycosylated at the Fc region amino acid corresponding to position 297. Such antibodies lack Fc effector function.

Thus, in some embodiments of the invention, the TCR binding protein does not exhibit Fc-mediated binding to an effector ligand such as an Fc receptor or the C1 component of complement due to the absence of the Fc domain in the binding protein while, in other cases, the lack of binding or effector function is due to an alteration in the constant region of the antibody.

In certain embodiments, the TCR binding proteins that can be used in the practice of the invention include proteins comprising a CDR sequence derived from (i.e., having a sequence the same as or similar to) the CDRs of the mouse monoclonal antibody BMA 031. Complementary cDNAs encoding the heavy chain and light chain variable regions of the mouse BMA 031 monoclonal antibody, including the CDR encoding sequences, may be cloned and sequenced accordingly to methods commonly known in the art. The amino acid sequences of BMA031 are provided herein and are designated SEQ ID NO:1 (for the VH) and SEQ ID NO:7 (for the VL). Using the mouse variable region and CDR sequences, a large number of chimeric and humanized monoclonal antibodies, comprising complementary determining regions derived from BMA 031 CDRs may be produced and analyzed. To identify humanized antibodies that bind TCR and, in particular, the extracellular constant domain of the TCR α chain, with high affinity and have other desirable properties, antibody heavy chains comprising a VH region with CDRs derived from BMA 031 may be produced and combined (by coexpression) with antibody light chains comprising a VL region with CDRs derived from BMA 031 to produce a tetrameric antibody for analysis. Properties of the resulting tetrameric antibodies may be assessed by any method known in the art and/or described herein. TCR binding proteins comprising CDRs derived from those of BMA 031 may be used according to the invention to manage, treat, or ameliorate one of the symptoms of an autoimmune disease and/or a T cell malignancy.

As is known in the art and described herein, immunoglobulin light and heavy chains can be recombinantly expressed under conditions in which they associate to produce a tetrameric antibody, or can be so combined in vitro. Similarly, combinations of VL and/or VH domains can be expressed in the form of single chain antibodies, and still other TCR binding proteins that comprise a VL and/or VH domain can be expressed by known methods. It will thus be appreciated that a BMA 031-derived VL-domain can be combined with a BMA 031-derived VH-domain to produce a novel TCR binding protein.

Sequences of VL and VH domains derived according to the methods presented herein can be further modified by art-known methods such as affinity maturation (see Schier et al., 1996, J. Mol. Biol. 263:551-67; Daugherty et al., 1998, Protein Eng. 11:825-32; Boder et al., 1997, Nat. Biotechnol. 15:553-57; Boder et al., 2000, Proc. Natl. Acad. Sci. U.S.A 97:10701-705; Hudson and Souriau, 2003, Nature Medicine 9:129-39). For example, the TCR binding proteins can be modified using affinity maturation techniques to identify proteins with increased affinity for TCR elative to the parent BMA 031.

5.1 Humanized Antibodies

In preferred embodiments, the anti-TCR antibodies of the invention are human or humanized antibodies. A humanized antibody is an antibody, a variant or a fragment thereof which is capable of binding to a predetermined antigen and which comprises a framework region having substantially the amino acid sequence of a human immunoglobulin and a CDR having substantially the amino acid sequence of a non-human immunoglobulin. A humanized TCR specific antibody may comprise substantially all of at least one, and typically two, variable domains in which all or substantially all of the CDR regions correspond to those of a non-human immunoglobulin (i.e., donor antibody, e.g., from the murine anti-TCR antibody BMA 031) and all or substantially all of the framework regions are those of a human immunoglobulin consensus sequence. A humanized antibody of the invention may also comprises at least a portion of an immunoglobulin constant region (Fc), typically that of a human immunoglobulin. The constant domains of the humanized antibodies of the invention may be selected with respect to the proposed function of the antibody, in particular the effector function which may be required. In some embodiments, the constant domains of the humanized antibodies of the invention are human IgA, IgE, IgG or IgM domains. In a specific embodiment, human IgG constant domains, especially of the IgG1 and IgG3 isotypes are used, when the humanized antibodies of the invention is intended for therapeutic uses and antibody effector functions are needed. In alternative embodiments, IgG2 and IgG4 isotypes are used when the humanized antibody of the invention is intended for therapeutic purposes and antibody effector function is not required.

The antibody of the invention may be a single domain antibody in that it comprises only a heavy chain variable domain or a light chain variable domain. In some embodiments, the antibody contains both the light chain as well as at least the variable domain of a heavy chain. In other embodiments, the antibody may further comprise one or more of the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. In some embodiments, the constant domain is a complement fixing constant domain where it is desired that the humanized antibody exhibit cytotoxic activity, and the class is typically IgG₁. In other embodiments, where such cytotoxic activity is not desirable, the constant domain may be of the IgG₂ class. The humanized antibody may comprise sequences from more than one class or isotype, and selecting particular constant domains to optimize desired effector functions is within the ordinary skill in the art.

The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the donor antibody. Such mutations, however, are preferably not extensive. Usually, at least 75% of the humanized antibody residues will correspond to those of the parental framework region (FR) and CDR sequences, more often 90%, and most preferably greater than 95%. Humanized antibodies can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (U.S. Pat. Nos. 5,770,196; 5,776,866; 5, 821,123; and 5,896,619; European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, Proc Natl Acad Sci USA 91:969-973), chain shuffling (U.S. Pat. No. 5,565,332), and techniques disclosed in, e.g., U.S. Patent Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morea et al., 2000, Methods 20:267-79, Baca et al., 1997, J. Biol. Chem. 272:10678-84, Roguska et al., 1996, Protein Eng. 9:895-904, Couto et al., 1995, Cancer Res. 55 (23 Supp):5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., 1988, Nature 332:323, all of which are incorporated herein by reference in their entireties.)

The present invention provides for the use of humanized antibody molecules specific for TCR, preferably the constant domain of the TCR α chain, in which one or more regions of one or more CDRs of the heavy and/or light chain variable regions of a human antibody (the recipient antibody) have been substituted by analogous parts of one or more CDRs of a donor monoclonal antibody which specifically binds TCR, e.g., the murine monoclonal antibody BMA031. In specific embodiments, the humanized antibodies bind to the same epitope as the BMA031 antibody. In a most preferred embodiment, the humanized antibody specifically binds to the same epitope as the donor murine antibody. It will be appreciated by one skilled in the art that the invention encompasses CDR grafting of antibodies in general. Thus, the donor and acceptor antibodies may be derived from animals of the same species and even same antibody class or sub-class. More usually, however, the donor and acceptor antibodies are derived from animals of different species. Typically the donor antibody is a non-human antibody, such as a rodent MAb, and the acceptor antibody is a human antibody.

In some embodiments, at least one CDR from the donor antibody is grafted onto the human antibody. In other embodiments, at least two and preferably all three CDRs of each of the heavy and/or light chain variable regions are grafted onto the human antibody. The CDRs may comprise the Kabat CDRs, the structural loop CDRs or a combination thereof. In some embodiments, the invention encompasses a humanized TCR antibody comprising at least one CDR grafted heavy chain and at least one CDR-grafted light chain.

In a preferred embodiment, the CDR regions of the humanized TCR specific antibody are derived from a murine antibody specific for TCR. In some embodiments, the humanized antibodies described herein comprise alterations, including but not limited to amino acid deletions, insertions, modifications, of the acceptor antibody, i.e., human, heavy and/or light chain variable domain framework regions that are necessary for retaining binding specificity of the donor monoclonal antibody. In some embodiments, the framework regions of the humanized antibodies described herein does not necessarily consist of the precise amino acid sequence of the framework region of a natural occurring human antibody variable region, but contains various alterations, including but not limited to amino acid deletions, insertions, modifications that alter the property of the humanized antibody, for example, improve the binding properties of a humanized antibody region that is specific for the same target as the murine TCR specific antibody. In most preferred embodiments, a minimal number of alterations are made to the framework region in order to avoid large-scale introductions of non-human framework residues and to ensure minimal immunogenicity of the humanized antibody in humans. The donor monoclonal antibody is preferably the murine monoclonal antibody BMA 031.

In a specific embodiment, the invention encompasses the use of a humanized antibody that specifically binds TCR, wherein the humanized antibody comprises a heavy chain variable region domain comprising framework residues of the recipient antibody and residues from the donor monoclonal antibody, which specifically binds TCR, e.g., monoclonal antibody BMA 031, 3H2906, IP26, T10B9, WT31, G-11, H-197, H-41, A-20, FL-189, H-20, H-250, C-17, H57, KJ1-26, KJ12-98, H28-710, UC3-10A6, GL3, 3H2928, 5K24, 3A10 or UC7-13D5. In another specific embodiment, the invention encompasses the use of a CDR-grafted antibody which specifically binds TCR, wherein the CDR-grafted antibody comprises a light chain variable region domain comprising framework residues of the recipient antibody and residues from the donor monoclonal antibody, which specifically binds TCR, e.g., monoclonal antibody BMA 031, 3H2906, IP26, T10B9, WT31, G-11, H-197, H-41, A-20, FL-189, H-20, H-250, C-17, H57, KJ1-26, KJ12-98, H28-710, UC3-10A6, GL3, 3H2928, 5K24, 3A10 or UC7-13D5. The antibody of the invention may bind the extracellular domain of recombinant or, preferably, native human TCR. As used herein, the term “native TCR” refers to a TCR complex that is endogenously expressed and present on the surface of a cell. In some embodiments, “native TCR” encompasses a protein that is recombinantly expressed in a mammalian cell. Preferably, the native TCR is not expressed in a bacterial cell, i.e., E. coli. Most preferably the native TCR is not denatured, i.e., it is in its biologically active conformation.

In one specific embodiment, a humanized TCR antibody is provided, wherein the VH region consists of the FR segments from the human germline VH segment VH1-18 (Matsuda et al., 1998, J. Exp. Med. 188:2151062) and JH6 (Ravetch et al., 1981, Cell 27(3 Pt. 2): 583-91), and one or more VH CDR regions of a murine anti TCR antibody (e.g., BMA 031, 3H2906, IP26, T10B9, WT31, G-11, H-197, H-41, A-20, FL-189, H-20, H-250, C-17, H57, KJ1-26, KJ12-98, H28-710, UC3-10A6, GL3, 3H2928, 5K24, 3A10 or UC7-13D5). In another specific embodiment, the humanized TCR antibody further comprises a VL region, which consists of the FR segments of the human germline VL segment VK-A26 (Lautner-Rieske et al., 1992, Eur. J. Immunol. 22:1023-1029) and JK4 (Hieter et al., 1982, J. Biol. Chem. 257:1516-22), and one or more VL CDR regions of a murine anti TCR antibody (e.g., BMA 031, BMA 031, 3H2906, IP26, T10B9, WT31, G-11, H-197, H-41, A-20, FL-189, H-20, H-250, C-17, H57, KJ1-26, KJ12-98, H28-710, UC3-10A6, GL3, 3H2928, 5K24, 3A10 or UC7-13D5).

In particular, a humanized antibody is provided that immunospecifically binds to human TCR, preferably the extracellular constant domain of the TCR α chain, said antibody comprising (or alternatively, consisting of) CDR sequences of BMA 031, 3H2906, IP26, T10B9, WT31, G-11, H-197, H-41, A-20, FL-189, H-20, H-250, C-17, H57, KJ1-26, KJ12-98, H28-710, UC3-10A6, GL3, 3H2928, 5K24, 3A10 or UC7-13D5, in any of the following combinations: a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs disclosed herein.

5.2 Human Antibodies

The present invention also provides for human anti-TCR antibodies. Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized using conventional methodologies with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93, which is incorporated herein by reference in its entirety). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

5.3 Chimeric Antibodies

The present invention also provides for a chimeric anti-TCR antibody, e.g., comprising the heavy chain and/or light chain variable domain from murine antibody BMA 031 (the heavy chain and light chain variable domains of BMA 031 are provided in SEQ ID NO:1 and SEQ ID NO:6, respectively). A chimeric antibody is a molecule in which different portions of the antibody are derived from different immunoglobulin molecules such as antibodies having a variable region derived from a non-human antibody and a human immunoglobulin constant region. The present invention provides chimeric antibodies derived from antibodies produced from known anti-TCR antibodies (e.g., BMA 031, 3H2906, IP26, T10B9, WT31, G-11, H-197, H-41, A-20, FL-189, H-20, H-250, C-17, H57, KJ1-26, KJ12-98, H28-710, UC3-10A6, GL3, 3H2928, 5K24, 3A10 and UC7-13D5). Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 6,311,415, 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNAS 91:969), and chain shuffling (U.S. Pat. No. 5,565,332). Each of the above-identified references is incorporated herein by reference in its entirety.

5.4 Fc Region Modifications

The invention encompasses antibodies with Fc constant domains comprising one or more amino acid modifications which alter antibody effector functions such as those disclosed in U.S. Patent Application Publication Nos. U.S. 2005/0037000 and U.S. 2005/0064514; U.S. Pat. Nos. 5,624,821 and 5,648,260 and European Patent No. EP 0 307 434; all of which are incorporated herein by reference in their entireties. These antibodies may exhibit improved ADCC activity (i.e., 2-fold, 10-fold, 100-fold, 500-fold, etc.) compared to comparable antibodies without amino acid modification.

The present invention encompasses antibodies comprising modifications preferably, in the Fc region that modify the binding affinity of the antibody to one or more FcγR. Methods for modifying antibodies with modified binding to one or more FcγR are known in the art, see, e.g., PCT Publication Nos. WO 04/029207, WO 04/029092, WO 04/028564, WO 99/58572, WO 99/51642, WO 98/23289, WO 89/07142, WO 88/07089, and U.S. Pat. Nos. 5,843,597 and 5,642,821, each of which is incorporated herein by reference in their entirety. In some embodiments, the invention encompasses antibodies that have altered affinity for an activating FcγR, e.g., FcγRIIIA. Preferably such modifications also have an altered Fc-mediated effector function. Modifications that affect Fc-mediated effector function are known in the art (See U.S. Pat. No. 6,194,551, which is incorporated herein by reference in its entirety). See International Publication No. WO 00/42072 and U.S. Pat. No. 6,194,551 which are incorporated herein by reference in their entirety.

Modification of the Fc region of the antibody of the invention allows the selection or modulation of Fc mediated effector function according to therapeutic use as understood in the art. For example, in certain embodiments, the invention provides for the use of the antibodies of the invention for the prevention or treatment of a T cell cancer or malignancy. In such embodiments, the desired therapeutic outcome may be a depletion of the cancerous or malignant T-cells. One means to effect such depletion is to modify the Fc region to increase binding to one or more Fc effector ligand relative to a wild type Fc region and thereby improve/increase the engagement/activation of Fc mediated cytotoxic mechanisms (e.g., activation of the complement system, or of the cytotoxic activity of macrophages, monocytes, or other antibody dependent cytotoxic cells). In contrast, in other embodiments, it may be desirable to decrease or abrogate Fc mediated effector function; examples of such embodiments may be in the use of the antibody for the treatment or prevention of an autoimmune disease, or the amelioration of one or more symptom thereof as described herein or as known in the art.

In one particular embodiment, the modification of the Fc region comprises one or more mutations in the Fc region. The one or more mutations in the Fc region may result in an antibody with an altered antibody-mediated effector function, an altered binding to other Fc receptors (e.g., Fc activation receptors), an altered ADCC activity, or an altered C1q binding activity, or an altered complement dependent cytotoxicity activity, or any combination thereof.

In some embodiments, the invention encompasses molecules comprising a variant Fc region having an amino acid modification at one or more of the following positions: 119, 125, 132, 133, 141, 142, 147, 149, 162, 166, 185, 192, 202, 205, 210, 214, 215, 216, 217, 218, 219, 221, 222, 223, 224, 225, 227, 229, 231, 232, 233, 235, 240, 241, 242, 243, 244, 246, 247, 248, 250, 251, 252, 253, 254, 255, 256, 258, 261, 262, 263, 268, 269, 270, 272, 274, 275, 276, 279, 280, 281, 282, 284, 287, 288, 289, 290, 291, 292, 293, 295, 298, 301, 303, 304, 305, 306, 307, 308, 309, 310, 311, 312, 313, 315, 316, 317, 318, 319, 320, 323, 326, 327, 328, 330, 333, 334, 335, 337, 339, 340, 343, 344, 345, 347, 348, 352, 353, 354, 355, 358, 359, 360, 361, 362, 365, 366, 367, 369, 370, 371, 372, 375, 377, 378, 379, 380, 381, 382, 383, 384, 385, 386, 387, 388, 389, 390, 392, 393, 394, 395, 396, 397, 398, 399, 400, 401, 402, 404, 406, 407, 408, 409, 410, 411, 412, 414, 415, 416, 417, 419, 420, 421, 422, 423, 424, 427, 428, 431, 433, 435, 436, 438, 440, 441, 442, 443, 446, or 447.

The invention encompasses molecules comprising variant Fc regions consisting of or comprising any of the mutations listed in the table below in Table 2.

TABLE 2
EXEMPLARY MUTATIONS

| SINGLE SITE MUTANTS | DOUBLE SITE MUTANTS |
|---|---|
| K392R | Q347H, A339V |
| N315I | S415I, L251F |
| S132I | K290E, L142P |
| P396L | G285E, P247H |
| P396H | K409R, S166N |
| A162V | E334A, K334A |
| R292L | R292L. K334E |
| T359N | K288N, A330S |
| T366S | R255L, E318K |
| V379L | F243L, E318K |
| K288N | V279L, P395S |
| A330S | K246T, Y319F |
| F243L | F243I, V379L |
| E318K | K288M, K334E |
| V379M | K334E, E308D |
| S219Y | E233D, K334E |
| V282M | K246T, P396H |
| D401V | H268D, E318D |
| K222N | K246I, K334N |
| K334I | K320E, K326E |
| K334E | S375C, P396L |
| I377F | K288N, K326N |
| P247L | P247L, N421K |
| F372Y | S298N, W381R |
| K326E | R255Q, K326E |
| H224L | V284A, F372L |
| F275Y | T394M. V397M |
| L398V | P247L, E389G |
| K334N | K290T, G371D |
| S400P | P247L, L398Q |
| S407I | P247L, I377F |
| F372Y | K326E, G385E |
| T366N | S298N, S407R |
| K414N | E258D, N384K |
| M352L | F241L, E258G |
| T225S | K370N, S440N |
| I377N | K317N, F423-DELETED |
| K248M | P227S, K290E |
| R292G | K334E, E380D |
| S298N | P291S, P353Q |
| D270E | V240I, V281M |
| E233G | P232S, S304G |
| | P247L, L406F |
| | D399E, M428L |
| | L251F, F372L |
| | D399E, G402D |
| | D399E, M428L |
| | K392T, P396L |
| | H268N, P396L |
| | K326I, P396L |
| | H268D, P396L |
| | K210M, P396L |
| | L358P, P396L |
| | K334N, P396L |
| | V379M, P396L |
| | P227S, P396L |
| | P217S, P396L |
| | Q419H, P396L |
| | K370E, P396L |
| | L242F, P396L |
| | R255L, P396L |
| | V240A, P396L |
| | T250A, P396L |
| | P247S, P396L |
| | L410H, P396L |
| | Q419L, P396L |
| | V427A, P396L |
| | E258D, P396L |
| | N384K, P396L |
| | V323I, P396L |
| | P244H, P396L |
| | V305L, P396L |
| | S400F, P396L |
| | V303I, P396L |
| | A330V, Q419H |
| | V263Q, E272D |
| | K326E, A330T |

In yet other embodiments, the invention encompasses molecules comprising variant Fc regions having more than two amino acid modifications. A non-limiting example of such variants is listed in the table below (Table 3). The invention encompasses mutations listed in Table 3 which further comprise one or more amino acid modifications such as those disclosed herein.

TABLE 3
EXEMPLARY COMBINATION VARIANTS

D399E, R292L, V185M
R301C, M252L, S192T
P291S, K288E, H268L, A141V
S383N, N384K, T256N, V262L, K218E, R214I, K205E, F149Y, K133M
S408I, V215I, V125L
G385E, P247H
V348M, K334N, F275I, Y202M, K147T
H310Y, T289A, Y407V, E258D
R292L, P396L, T359N
F275I, K334N, V348M
F243L. R255L, E318K
K334E, T359N, T366S
T256S, V305I, K334E, N390S
T335N, K370E, A378V, T394M, S424L
K334E, T359N, T366S, Q386R
K288N, A330S, P396L
P244H, L358M, V379M, N384K, V397M
P217S, A378V, S408R
P247L, I253N, K334N
D312E, K327N, I378S
D280E, S354F, A431D, L441I
K218R, G281D, G385R
P247L, A330T, S440G
T355N, P387S, H435Q
P247L, A431V, S442F
P343S, P353L, S375I, S383N
E216D, E345K, S375I
K288N, A330S, P396L
K222N, T335N, K370E, A378V, T394M
G316D, A378V, D399E
N315I, V379M, T394M
K326Q, K334E, T359N, T366S
A378V, N390I, V422I
V282E, V369I, L406F
V397M, T411A, S415N
T223I, T256S, L406F
L235P, V382M, S304G, V305I, V323I
P247L, W313R, E388G
D221Y, M252I, A330G, A339T, T359N, V422I, H433L
F243I, V379L, G420V
A231V, Q386H, V412M
T215P, K274N, A287G, K334N, L365V, P396L
P244A, K326I, C367R, S375I, K447T
R301H, K340E, D399E
C229Y, A287T, V379M, P396L, L443V
E269K, K290N, Q311R, H433Y
E216D, K334R, S375I
T335N, P387S, H435Q
K246I, Q362H, K370E
K334E, E380D, G446V
V303I, V369F, M428L
K246E, V284M, V308A
E293V, Q295E, A327T
Y319F, P352L, P396L
D221E, D270E, V308A, Q311H, P396L, G402D
K290T, N390I, P396L
K288R, T307A, K344E, P396L
V273I, K326E, L328I, P396L
K326I, S408N, P396L
K261N, K210M, P396L
F243L, V305I, A378D, F404S, P396L
K290E, V369A, T393A, P396L
K210N, K222I, K320M, P396L
P217S, V305I, I309L, N390H, P396L
K246N, Q419R, P396L
P217A, T359A, P396L
V215I, K290V, P396L
F275L, Q362H, N384K, P396L
A330V, H433Q, V427M
V263Q, E272D, Q419H
N276Y, T393N, W417R
V282L, A330V, H433Y, T436R
V284M, S298N, K334E, R355W
A330V, G427M, K438R
S219T, T225K, D270E, K360R
K222E, V263Q, S298N
E233G, P247S, L306P
S219T, T225K, D270E
S254T, A330V, N361D, P243L
V284M, S298N, K334E, R355W R416T
D270E, G316D, R416G
K392T, P396L, D270E
R255L, P396L, D270E
V240A, P396L, D270E
Q419H, P396L, D270E
K370E, P396L, D270E
P247L, N421K, D270E
R292P, V305I
R292P, V305I, F243L
V284M, R292L, K370N
F243L, R292L, Y300L

5.5 Carbohydrate Modifications

The invention also provides antibodies with altered oligosaccharide content. Oligosaccharides as used herein refer to carbohydrates containing two or more simple sugars and the two terms may be used interchangeably herein. Carbohydrate moieties of the instant invention will be described with reference to commonly used nomenclature in the art. For a review of carbohydrate chemistry, see, e.g., Hubbard et al., 1981 Ann. Rev. Biochem., 50: 555-583, which is incorporated herein by reference in its entirety. This nomenclature includes for example, Man which represents mannose; GlcNAc which represents 2-N-acetylglucosamine; Gal which represents galactose; Fuc for fucose and Glc for glucose. Sialic acids are described by the shorthand notation NeuNAc for 5-N-acetylneuraminic acid, and NeuNGc for 5-glycolneuraminic.

In general, antibodies contain carbohydrate moeities at conserved positions in the constant region of the heavy chain, and up to 30% of human IgGs have a glycosylated Fab region. IgG has a single N-linked biantennary carbohydrate structure at Asn 297 which resides in the CH2 domain (Jefferis et al., 1998, Immunol. Rev. 163: 59-76; Wright et al., 1997, Trends Biotech 15: 26-32). Human IgG typically has a carbohydrate of the following structure; GlcNAc(Fucose)-GlcNAc-Man-(ManGlcNAc)₂. However variations among IgGs in carbohydrate content does occur which leads to altered function, see, e.g., Jassal et al., 2001 Biochem. Biophys. Res. Commun. 288: 243-9; Groenink et al., 1996 J. Immunol. 26: 1404-7; Boyd et al., 1995 Mol. Immunol. 32: 1311-8; Kumpel et al., 1994, Human Antibody Hybridomas, 5: 143-51. The invention encompasses antibodies comprising a variation in the carbohydrate moiety that is attached to Asn 297. In one embodiment, the carbohydrate moiety has a galactose and/or galactose-sialic acid at one or both of the terminal GlcNAc and/or a third GlcNac arm (bisecting GlcNAc).

In some embodiments, the antibodies of the invention are substantially free of one or more selected sugar groups, e.g., one or more sialic acid residues, one or more galactose residues, one or more fucose residues. An antibody that is substantially free of one or more selected sugar groups may be prepared using common methods known to one skilled in the art, including for example recombinantly producing an antibody of the invention in a host cell that is defective in the addition of the selected sugar groups(s) to the carbohydrate moiety of the antibody, such that about 90-100% of the antibody in the composition lacks the selected sugar group(s) attached to the carbohydrate moiety. Alternative methods for preparing such antibodies include for example, culturing cells under conditions which prevent or reduce the addition of one or more selected sugar groups, or post-translational removal of one or more selected sugar groups.

In a specific embodiment, the invention encompasses a method of producing a substantially homogenous antibody preparation, wherein about 80-100% of the antibody in the composition lacks a fucose on its carbohydrate moiety, e.g., the carbohydrate attachment on Asn 297. The antibody may be prepared for example by (a) use of an engineered host cell that is deficient in fucose metabolism such that it has a reduced ability to fucosylate proteins expressed therein; (b) culturing cells under conditions which prevent or reduce fusocylation; (c) post-translational removal of fucose, e.g., with a fucosidase enzyme; or (d) purification of the antibody so as to select for the product which is not fucosylated. Most preferably, nucleic acid encoding the desired antibody is expressed in a host cell that has a reduced ability to fucosylate the antibody expressed therein. Preferably the host cell is a dihydrofolate reductase deficient chinese hamster ovary cell (CHO), e.g., a Lec 13 CHO cell (lectin resistant CHO mutant cell line; Ribka & Stanley, 1986, Somatic Cell & Molec. Gen. 12(1): 51-62; Ripka et al., 1986 Arch. Biochem. Biophys. 249(2): 533-45), CHO-K1, DUX-B11, CHO-DP12 or CHO-DG44, which has been modified so that the antibody is not substantially fucosylated. Thus, the cell may display altered expression and/or activity for the fucoysltransferase enzyme, or another enzyme or substrate involved in adding fucose to the N-linked oligosaccharide so that the enzyme has a diminished activity and/or reduced expression level in the cell. For methods to produce antibodies with altered fucose content, see, e.g., WO 03/035835 and Shields et al., 2002, J. Biol. Chem. 277(30): 26733-40; both of which are incorporated herein by reference in their entirety.

In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for e.g., see Shields R. L. et al., 2001, J. Biol. Chem. 277(30): 26733-40; Davies J. et al., 2001, Biotechnology & Bioengineering, 74(4): 288-294). In another specific embodiment, the altered carbohydrate modifications enhance the binding of antibodies of the invention to TCR, or any peptide component thereof. Altering carbohydrate modifications in accordance with the methods of the invention includes, for example, increasing the carbohydrate content of the antibody or decreasing the carbohydrate content of the antibody. Methods of altering carbohydrate contents are known to those skilled in the art, see, e.g., Wallick et al., 1988, Journal of Exp. Med. 168(3): 1099-1109; Tao et al., 1989 Journal of Immunology, 143(8): 2595-2601; Routledge et al., 1995 Transplantation, 60(8): 847-53; Elliott et al. 2003; Nature Biotechnology, 21: 414-21; Shields et al. 2002 Journal of Biological Chemistry, 277(30): 26733-40; all of which are incorporated herein by reference in their entirety.

In some embodiments, the invention encompasses antibodies comprising one or more glycosylation sites, so that one or more carbohydrate moieties are covalently attached to the antibody. In other embodiments, the invention encompasses antibodies comprising one or more glycosylation sites and one or more modifications in the Fc region, such as those disclosed supra and those known to one skilled in the art. In preferred embodiments, the one or more modifications in the Fc region enhance the affinity of the antibody for an activating FcγR, e.g., FcγRIIIA, relative to the antibody comprising the wild type Fc regions. Antibodies of the invention with one or more glycosylation sites and/or one or more modifications in the Fc region have an enhanced antibody mediated effector function, e.g., enhanced ADCC activity. In some embodiments, the invention further comprises antibodies comprising one or more modifications of amino acids that are directly or indirectly known to interact with a carbohydrate moiety of the antibody, including but not limited to amino acids at positions 241, 243, 244, 245, 245, 249, 256, 258, 260, 262, 264, 265, 296, 299, and 301. Amino acids that directly or indirectly interact with a carbohydrate moiety of an antibody are known in the art, see, e.g., Jefferis et al., 1995 Immunology Letters, 44: 111-7, which is incorporated herein by reference in its entirety.

The invention encompasses antibodies that have been modified by introducing one or more glycosylation sites into one or more sites of the antibodies, preferably without altering the functionality of the antibody, e.g., binding activity to TCR. Glycosylation sites may be introduced into the variable and/or constant region of the antibodies of the invention. As used herein, “glycosylation sites” include any specific amino acid sequence in an antibody to which an oligosaccharide (i.e., carbohydrates containing two or more simple sugars linked together) will specifically and covalently attach. Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. The antibodies of the invention may comprise one or more glycosylation sites, including N-linked and O-linked glycosylation sites. Any glycosylation site for N-linked or O-linked glycosylation known in the art may be used in accordance with the instant invention. An exemplary N-linked glycosylation site that is useful in accordance with the methods of the present invention, is the amino acid sequence: Asn-X-Thr/Ser, wherein X may be any amino acid and Thr/Ser indicates a threonine or a serine. Such a site or sites may be introduced into an antibody of the invention using methods well known in the art to which this invention pertains. See, for example, “In vitro Mutagenesis,” Recombinant DNA: A Short Course, J. D. Watson, et al. W.H. Freeman and Company, New York, 1983, chapter 8, pp. 106-116, which is incorporated herein by reference in its entirety. An exemplary method for introducing a glycosylation site into an antibody of the invention may comprise: modifying or mutating an amino acid sequence of the antibody so that the desired Asn-X-Thr/Ser sequence is obtained.

In some embodiments, the invention encompasses methods of modifying the carbohydrate content of an antibody of the invention by adding or deleting a glycosylation site. Methods for modifying the carbohydrate content of antibodies are well known in the art and encompassed within the invention, see, e.g., U.S. Pat. No. 6,218,149; EP 0 359 096 B1; U.S. Publication No. US 2002/0028486; WO 03/035835; U.S. Publication No. 2003/0115614; U.S. Pat. No. 6,218,149; U.S. Pat. No. 6,472,511; all of which are incorporated herein by reference in their entirety. In other embodiments, the invention encompasses methods of modifying the carbohydrate content of an antibody of the invention by deleting one or more endogenous carbohydrate moieties of the antibody.

In some specific embodiments, the invention encompasses the use of modified TCR antibodies wherein the N-glycosylation consensus site Asn₅₀-Val-Ser of the CDR2 region has been modified, so that the glycosylation site at position 50 is eliminated. Although not intending to be bound by a particular mechanism of action, removal of the glycosylation site may limit potential variation in production of the antibody as well as potential immunogenicity in a pharmaceutical application. In a specific embodiment, the invention encompasses the use of a humanized TCR antibody wherein the amino acid at position 50 has been modified, e.g., deleted or substituted. In another specific embodiment, the invention further encompasses the use of an antibody with an amino acid modification, e.g., deletion or substitution, at position 51. In one specific embodiment, the invention encompasses the use of a humanized TCR antibody wherein the amino acid at position 50 has been replaced with tyrosine. In another more specific embodiment, the invention encompasses the use of a TCR antibody wherein the amino acid at position 50 has been replaced with tyrosine and the amino acid at position 51 has been replaced with alanine

5.6 Antibody Conjugates

The present invention encompasses antibodies recombinantly fused or chemically conjugated (including both covalently and non-covalently conjugations) to heterologous polypeptides (i.e., an unrelated polypeptide; or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. Antibodies may be used for example to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., PCT Publication No. WO 93/21232; EP 439,095; Naramura et al., 1994, Immunol. Lett., 39:91-99; U.S. Pat. No. 5,474,981; Gillies et al., 1992, Proc Natl Acad Sci, 89:1428-1432; and Fell et al., 1991, J. Immunol., 146:2446-2452, each of which is incorporated herein by reference in their entireties.

Further, an antibody may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin (i.e., PE-40), or diphtheria toxin, ricin, gelonin, and pokeweed antiviral protein, a protein such as tumor necrosis factor, interferons including, but not limited to, α-interferon (IFN-α), β-interferon (IFN-β), nerve growth factor (NGF), platelet derived growth factor (PDGF), tissue plasminogen activator (TPA), an apoptotic agent (e.g., TNF-α, TNF-β, AIM I as disclosed in PCT Publication No. WO 97/33899), AIM II (see, e.g., PCT Publication No. WO 97/34911), Fas Ligand (Takahashi et al., 1994 J. Immunol., 6:1567-1574), and VEGI (PCT Publication No. WO 99/23105), a thrombotic agent or an anti-angiogenic agent (e.g., angiostatin or endostatin), or a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), macrophage colony stimulating factor, (“M-CSF”), or a growth factor (e.g., growth hormone (“GH”); a protease, or a ribonuclease).

Antibodies can be fused to marker sequences, such as a peptide, to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989 Proc. Natl. Acad. Sci. USA, 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984 Cell, 37:767) and the “flag” tag (Knappik et al., 1994 Biotechniques, 17(4):754-761).

The present invention also includes the use of compositions comprising heterologous polypeptides fused or conjugated to antibodies of the invention or fragments thereof. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to TCR, or any polypeptide component thereof, may be recombined with one or more components, motifs, sections, parts, domains, fragments, etc. of one or more heterologous molecules.

The present invention also encompasses antibodies conjugated to a diagnostic or therapeutic agent or any other molecule for which serum half-life is desired to be increased. The antibodies can be used diagnostically to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Such diagnosis and detection can be accomplished by coupling the antibody to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵¹Cr), cobalt (⁵⁷Co), fluorine (⁸F) gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹ I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

An antibody may be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Moreover, an antibody can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Techniques for conjugating such therapeutic moieties to antibodies are well known; see, e.g., Arnon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al., Immunol. Rev., 62:119-58, 1982.

An antibody or fragment thereof, with or without a therapeutic moiety conjugated to it, administered alone or in combination with cytotoxic factor(s) and/or cytokine(s) can be used as a therapeutic.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

5.7 Amino Acid Variants

Amino acid sequence variants of the antibody molecules can be created such that they are substitutional, insertional or deletion variants. Deletion variants lack one or more residues of the native protein which are not essential for function or immunogenic activity. Insertional mutants typically involve the addition of material at a non-terminal point in the polypeptide. This may include the insertion of an immunoreactive epitope or simply a single residue. Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Once general areas of the gene are identified as encoding particular protein domains as described herein, point mutagenesis may be employed to identify with particularity which amino acid residues are important in particular activities associated with a particular function. Thus, one of skill in the art will be able to generate single base changes in the DNA strand to result in an altered codon and a missense mutation.

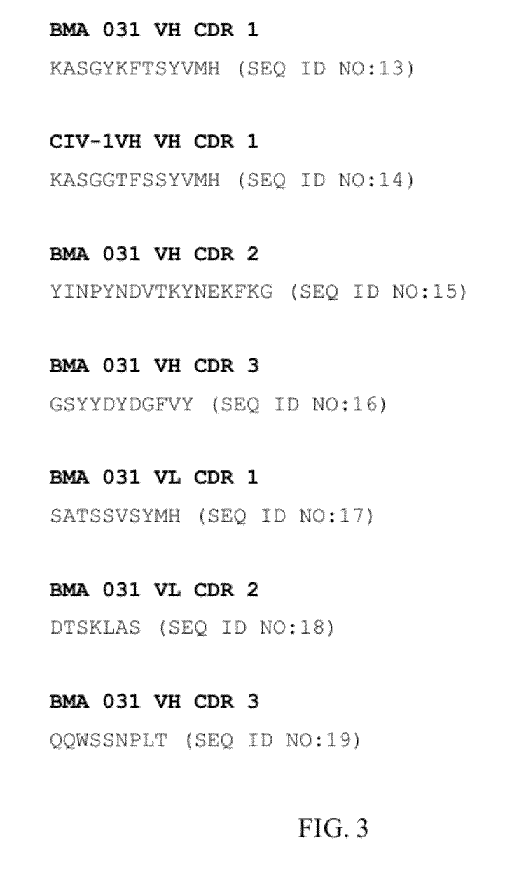

In certain embodiments, the TCR binding molecules of the invention comprise a CDR with 1, 2, 3, or 4 residue modifications (including substitution, deletion or insertion) relative to the corresponding CDR of BMA 031 (presented in FIG. 3). In other embodiments, the TCR binding molecules of the invention comprise a VH and/or VL domain having 1, 2, 3, 4, 5, 6, 7, 8, 9, 10, 11, 12, 13, 14, 15, 16, 17, 18, 19, 20, 21, 22, 23, or 24 residue modifications (including substitution, insertion or deletion) relative to the corresponding VH and/or VL domain of BMA 031. In still other embodiments, the TCR binding molecules of the invention comprise a VH domain having 1, 2, 3, 4, 5, 6, 7, 8, 9, 10, 11, 12, 13, 14, 15, 16, 17, 18, 19, 20, 21, 22, 23, or 24 residue modifications (including substitution, insertion or deletion) relative to the VH domain having the amino acid sequence of SEQ ID NO:2, SEQ ID NO:3, SEQ ID NO:4, SEQ ID NO:5 or SEQ ID NO:6. In yet other embodiments, the TCR binding molecules of the invention comprise a VL domain having 1, 2, 3, 4, 5, 6, 7, 8, 9, 10, 11, 12, 13, 14, 15, 16, 17, 18, 19, 20, 21, 22, 23, or 24 residue modifications (including substitution, insertion or deletion) relative to the VL domain having the amino acid sequence of SEQ ID NO:8, SEQ ID NO:9, SEQ ID NO:10, SEQ ID NO:11 or SEQ ID NO:12.

Preferably, mutation of the amino acids of a protein creates an equivalent, or even an improved, second-generation molecule. For example, certain amino acids may be substituted for other amino acids in a protein structure without detectable loss of affinity or avidity. In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics; for example: isoleucine(+4.5); valine(+4.2); leucine(+3.8); phenylalanine(+2.8); cysteine/cystine(+2.5); methionine(+1.9); alanine(+1.8); glycine(−0.4); threonine(−0.7); serine(−0.8); tryptophan 0.9); tyrosine(−1.3); proline(−1.6); histidine(−3.2); glutamate(−3.5); glutamine(−3.5); aspartate (−3.5); asparagine (−3.5); lysine (−3.9); and arginine (−4.5).

It is also understood in the art that the substitution of like amino acids can be made effectively on the basis of hydrophilicity. Like hydrophobicity, values of hydrophilicity have been assigned to each amino acid: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). Equivalent molecules may be obtained by substitution of one amino acid for another where their hydrophilicity indices are within ±2, preferably ±1, or most preferably ±5 of each other.

5.8 Characterization of Anti-TCR Therapeutic or Prophylactic Utility

TCR binding molecules may be characterized in a variety of ways. In particular, TCR binding molecules may be assayed for the ability to immunospecifically bind to a TCR polypeptide. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310) (each of these references is incorporated herein in its entirety by reference).

TCR binding molecules may be assayed for immunospecific binding to a TCR polypeptide and cross-reactivity with other polypeptides by any method known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the TCR binding molecule of interest to the cell lysate, incubating for a period of time (e.g., 1 to 4 hours) at 40° C., adding protein A and/or protein G sepharose beads to the cell lysate, incubating for about an hour or more at 40° C., washing the beads in lysis buffer and resuspending the beads in SDS/sample buffer. The ability of the TCR binding molecule of interest to immunoprecipitate a particular antigen can be assessed by, e.g., western blot analysis. One of skill in the art would be knowledgeable as to the parameters that can be modified to increase the binding of the TCR binding molecule to a TCR polypeptide and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with TCR binding molecule of interest (e.g., an antibody of interest) diluted in blocking buffer, washing the membrane in washing buffer, blocking the membrane with an antibody (which recognizes the TCR binding molecule) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the TCR polypeptide. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing TCR polypeptide, coating the well of a 96 well microtiter plate with the TCR polypeptide, adding the TCR binding molecule of interest conjugated to a detectable compound such as an enzymatic substrate (e.g., horseradish peroxidase or alkaline phosphatase) to the well and incubating for a period of time, and detecting the presence of the TCR polypeptide. In ELISAs the TCR binding molecule of interest does not have to be conjugated to a detectable compound; instead, an antibody (which recognizes the TCR binding molecule of interest) conjugated to a detectable compound may be added to the well. Further, instead of coating the well with the TCR polypeptide, the TCR binding molecule may be coated to the well. In this case, an antibody conjugated to a detectable compound may be added following the addition of the TCR polypeptide to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of a TCR binding molecule to a TCR polypeptide and the off-rate of an TCR binding molecule-TCR polypeptide interaction can be determined by competitive binding assays. One example of a competitive binding assay is a radioimmunoassay comprising the incubation of labeled TCR polypeptide (e.g., ³H or ¹²⁵I) with the TCR binding molecule of interest in the presence of increasing amounts of unlabeled TCR polypeptide, and the detection of the TCR binding molecule bound to the labeled TCR polypeptide. The affinity of a TCR binding molecule for a TCR polypeptide and the binding off-rates can be determined from the data by scatchard plot analysis. Competition with a second TCR binding molecule can also be determined using radioimmunoassays. In this case, a TCR polypeptide is incubated with a TCR binding molecule conjugated to a labeled compound (e.g., ³H or ¹²⁵I) in the presence of increasing amounts of a second unlabeled TCR binding molecule.

In a preferred embodiment, BIAcore kinetic analysis is used to determine the binding on and off rates of TCR binding molecules to a TCR polypeptide. BIAcore kinetic analysis comprises analyzing the binding and dissociation of a TCR polypeptide from chips with immobilized TCR binding molecules on their surface.

The TCR binding molecules, in particular anti-human TCR antibodies, and compositions of the invention can also be assayed for their ability to modulate T cell activation. T cell activation can be determined by measuring, e.g., changes in the level of expression of cytokines and/or T cell activation markers. Techniques known to those of skill in the art, including, but not limited to, immunoprecipitation followed by western blot analysis, ELISAs, flow cytometry, Northern blot analysis, and RT-PCR can be used to measure the expression cytokines and T cell activation markers. In a preferred embodiment, a TCR binding molecule or composition of the invention is tested for its ability to induce the expression of IFN-γ and/or IL-2.

The anti-TCR antibodies, and compositions of the invention can also be assayed for their ability to induce T cell signaling. The ability of an anti-TCR antibody or a composition of the invention induce T cell signaling can be assayed, e.g., by kinase assays and electrophoretic shift assays (EMSAs).

The anti-TCR antibodies, and compositions of the invention can be tested in vitro or in vivo for their ability to modulate T cell proliferation. For example, the ability of an anti-TCR antibody or a composition of the invention to modulate T cell proliferation can be assessed by, e.g., ³H-thymidine incorporation, trypan blue cell counts, and fluorescence activated cell sorting (FACS).

The anti-human TCR antibodies, and compositions of the invention can be tested in vitro or in vivo for their ability to induce cytolysis. For example, the ability of an anti-TCR antibody or a composition of the invention to induce cytolysis can be assessed by, e.g., ⁵¹Cr-release assays.

The anti-TCR antibodies, and compositions of the invention can be tested in vitro or in vivo for their ability to mediate the depletion of peripheral blood T cells. For example, the ability of an anti-TCR antibody or a composition of the invention to mediate the depletion of peripheral blood T cells can be assessed by, e.g., measuring T cell counts using flow cytometry analysis.

The anti-TCR antibodies, and compositions of the invention can be tested in vivo for their ability to mediate peripheral blood lymphocyte counts. For example, the ability of an anti-TCR antibody or a composition of the invention to mediate peripheral blood lymphocyte counts can be assessed by, e.g., obtaining a sample of peripheral blood from a subject, separating the lymphocytes from other components of peripheral blood such as plasma using, e.g., a Ficoll gradient, and counting the lymphocytes using trypan blue.

In some embodiments, the in vivo activity of the TCR antibodies of the invention is determined in xenograft human tumor models. Tumors may be established using any cancer cell line expressing TCR and/or an antigen thereof. In some embodiments, the tumors will be established with two cancer cell lines, wherein the first cancer cell line is characterized by a low expression of a said antigen and a second cancer cell line, wherein the second cancer cell line is characterized by a high expression of the same said antigen. Tumor clearance may then be determined using methods known to one skilled in the art, using an anti-TCR antibody which immunospecifically binds the antigen on the first and second cancer cell line, and an appropriate mouse model, e.g., a Balb/c nude mouse model (e.g., Jackson Laboratories, Taconic), with adoptively transferred human monocytes and MDMs as effector cells.

An exemplary method for testing the in vivo activity of an antibody of the invention may comprise the following: establishing a xenograft murine model using a cancer cell line characterized by the expression of TCR or an antigen thereof and determining the effect of an antibody of the invention in mediating tumor clearance. Preferably, the in vivo activity is tested parallel using two cancer cell lines, wherein the first cancer cell line is characterized by a expression of said antigen at low levels and a second cancer cell line, characterized by the same antigen expressed at a higher level relative to the first cancer cell line. These experiments will thus increase the stringency of the evaluation of the role of an antibody of the invention in tumor clearance. In order to establish the xenograft tumor models, 5×10⁶ viable cells, may be injected, e.g., s.c., into, e.g., 8, age and weight matched female nude athymic mice using for example Matrigel (Becton Dickinson). The estimated weight of the tumor may be determined by the formula: length×(width)²/2; and preferably does not exceed 3 grams. The antibodies may be injected at a dose of, e.g., 1-5 μg each per gram of mouse body weight (mbw). The initial injection will be followed by weekly injections of antibodies for 4-6 weeks thereafter at 2 μg/wk. A group of mice will receive no therapeutic antibody but will be injected with an irrelevant isotype control antibody. Mice may be placed in groups of 4 and monitored three times weekly.

The endpoint of the xenograft tumor models is determined based on the size of the tumors, weight of mice, survival time and histochemical and histopathological examination of the cancer, using methods known to one skilled in the art. Criteria for tumor growth may be abdominal distention, presence of palpable mass in the peritoneal cavity. Preferably estimates of tumor weight versus days after inoculation will be calculated. A comparison of the aforementioned criteria of mice in the treatment group compared to those in control groups will define the role of an antibody of the invention in enhancement of tumor clearance. Preferably, antibody-treated animals will be under observation for an additional 2 months after the control group.

Preferably, the antibodies of the invention have an enhanced efficacy in decreasing tumor relative to a cancer therapeutic antibody when administered at the same dose, e.g., 10 μg/g, over a time period of at least 14 days, at least 21 days, at least 28 days, or at least 35 days. In most preferred embodiments, the antibodies of the invention reduce tumor size by at least 10 fold, at least 100 fold, at least 1000 fold relative to administration of a cancer therapeutic antibody at the same dose. In yet another preferred embodiment, the antibodies of the invention completely abolish the tumor.

Several aspects of the pharmaceutical compositions or the anti-TCR antibodies of the invention are preferably tested in vitro, in a cell culture system, and in an animal model organism, such as a rodent animal model system, for the desired therapeutic activity prior to use in humans. For example, assays which can be used to determine whether administration of a specific pharmaceutical composition is indicated, include cell culture assays in which a patient tissue sample is grown in culture, and exposed to or otherwise contacted with a pharmaceutical composition, and the effect of such composition upon the tissue sample is observed. The tissue sample can be obtained by biopsy from the patient. This test allows the identification of the therapeutically most effective tumor-targeted bacteria and the therapeutically most effective therapeutic molecules) for each individual patient. In various specific embodiments, in vitro assays can be carried out with representative cells of cell types involved in an autoimmune or inflammatory disorder (e.g., T cells), to determine if a pharmaceutical composition of the invention has a desired effect upon such cell types.

In accordance with the invention, clinical trials with human subjects need not be performed in order to demonstrate the prophylactic and/or therapeutic efficacy of anti-TCR antibodies. In vitro and animal model studies using anti-TCR antibodies can be extrapolated to humans and are sufficient for demonstrating the prophylactic and/or therapeutic utility of said anti-TCR antibodies.

Anti-TCR antibodies can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, TCR binding molecules are tested in a mouse model system. Such model systems are widely used and well-known to the skilled artisan. TCR binding molecules can be administered repeatedly. Several aspects of the procedure may vary. Said aspects include the temporal regime of administering TCR binding molecules, and whether such agents are administered separately or as an admixture.

The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993), incorporated herein by reference in its entirety. A collagen-induced arthritis (CIA) is an animal model for the human autoimmune disease rheumatoid arthritis (RA) (Trenthorn et al., 1977, J. Exp. Med. 146:857). This disease can be induced in many “species by the administration of heterologous type II collagen (Courtenay et al., 1980, Nature 283:665; and Cathcart et at, 1986, Lab. Invest. 54:26). With respect to animal models of arthritis see, in addition, e.g., Holmdahl, R., 1999, Curr. Biol. 15:R528-530.

Additionally, animal models for inflammatory bowel disease can also be used to assess the efficacy of the anti-TCR antibodies or pharmaceutical compositions of invention (Kim et al., 1992, Scand. J. Gastroentrol. 27:529-537; Strober, 1985, Dig. Dis. Sci. 30(12 Suppl):3S-1OS). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for asthma can also be used to assess the efficacy of anti-TCR antibodies or pharmaceutical compositions of invention. An example of one such model is the marine adoptive transfer model in which aeroallergen provocation of TH 1 or TH2 recipient mice results in TH effector cell migration to the airways and is associated with an intense neutrophilic (TH 1) and eosinophilic (TH2) lung mucosal inflammatory response (Cohn et al., 1997, J. Exp. Med. 1861737-1747).

Animal models for autoimmune disorders can also be used to assess the efficacy of anti-TCR antibodies or pharmaceutical compositions of invention. Animal models for autoimmune disorders such as type 1 diabetes, thyroid autoimmunity, systemic lupus eruthematosus, and glomerulonephritis have been developed (Bluestone et al., 2004, PNAS 101:14622-14626; Flanders et al., 1999, Autoimmunity 29:235-246; Krogh et al., 1999, Biochimie 81:511-515; Foster, 1999, Semin. Nephrol. 19:12-24).

The efficacy of anti-TCR antibodies or pharmaceutical compositions of invention can also be tested in such autoimmune disorder models as an experimental allergic encephalomyelitis (EAE) model. EAE is an experimental autoimmune disease of the central nervous system (CNS) (Zamvil et al, 1990, Ann. Rev, Immunol. 8:579) and is a disease model for the human autoimmune condition, multiple sclerosis (MS). EAE is an example of a cell-mediated autoimmune disorder that is mediated via T cells. EAE is readily induced in mammalian species by immunizations of myelin basic protein (MBP) purified from the CNS or an encephalitogenic proteolipid (PLP). SJL/J mice are a susceptible strain of mice (H-2u) and, upon induction of EAE, these mice develop an acute paralytic disease and an acute cellular infiltrate is identifiable within the CNS. EAE spontaneously develops in MBP1-17 peptide-specific T cell receptor (TCR) transgenic mice (TgMBP+) of a RAG-1-deficient background (Lafaille et al., 1994, Cell 78:399).

Further, any assays known to those skilled in the art can be used to evaluate anti-TCR antibodies or the pharmaceutical compositions disclosed herein for autoimmune and/or inflammatory diseases.

The toxicity and/or efficacy of anti-TCR antibodies or pharmaceutical compositions of invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Anti-TCR antibodies that exhibit large therapeutic indices are preferred. While anti-TCR antibodies that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of anti-TCR antibodies for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Efficacy in preventing or treating an autoimmune disorder may be demonstrated, e.g., by detecting the ability of a anti-TCR antibodies or composition of the invention to reduce one or more symptoms of the autoimmune disorder, to reduce mean absolute lymphocyte counts, to decrease T cell activation, to decrease T cell proliferation, to reduce cytokine production, or to modulate one or more particular cytokine profiles. Efficacy in treating diabetes may be demonstrated, e.g. by detecting the ability of a anti-TCR antibodies or composition of the invention to reduce one or more symptoms of diabetes, to preserve the C-peptide response to MMTT, to reduce the level HA1 or HA1c, to reduce the daily requirement for insulin, or to decrease T cell activation in pancreatic islet tissue. Efficacy in preventing or treating an inflammatory disorder may be demonstrated, e.g., by detecting, the ability of a an anti-TCR antibody to reduce one or more symptoms of the inflammatory disorder, to decrease T cell activation, to decrease T cell proliferation, to modulate one or more cytokine profiles, to reduce cytokine production, to reduce inflammation of a joint, organ or tissue or to improve quality of life.

Changes in inflammatory disease activity may be assessed through tender and swollen joint counts, patient and physician global scores for pain and disease activity, and the ESRICRP. Progression of structural joint damage may be assessed by quantitative scoring of X-rays of hands, wrists, and feet (Sharp method). Changes in functional status in humans with inflammatory disorders may be evaluated using the Health Assessment Questionnaire (HAQ), and quality of life changes are assessed with the SF-36.

5.9 Methods of Producing Antibodies

Antibodies that immunospecifically bind to a TCR polypeptide can be produced by any method known in the art for the synthesis of antibodies, in particular, by chemical synthesis or preferably, by recombinant expression techniques.

Polyclonal antibodies that immunospecifically bind to an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T cell Hybridomas 563 681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a TCR antigen and once an immune response is detected, e.g., antibodies specific for a TCR antigen (preferably, an antigen of the extracellular constant domain of the TCR α chain) are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating antibodies by culturing a hybridoma cell secreting an antibody of the invention wherein, preferably, the hybridoma is generated by fusing splenocytes isolated from a mouse immunized with a TCR antigen with myeloma cells and then screening the hybridomas resulting from the fusion for hybridoma clones that secrete an antibody able to bind to a TCR antigen.

Antibody fragments which recognize specific TCR antigen (preferably, an antigen of the extracellular constant domain of the TCR α chain) may be generated by any technique known to those of skill in the art. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Single domain antibodies, for example, antibodies lacking the light chains, can be produced by methods well-known in the art. See Riechmann et al., 1999, J. Immuno. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

5.10 Anti-TCR Antibody Therapy

In certain embodiments, the course of treatment with an anti-TCR antibody according to the methods of the invention is repeated at 2 month, 4 month, 6 month, 8 month, 9 month, 10 month, 12 month, 15 month, 18 month, 24 month, 30 month, or 36 month intervals. In specific embodiments efficacy of the treatment with an anti-TCR antibody of the invention is determined as described herein or as is known in the art at 2 months, 4 months, 6 months, 9 months, 12 months, 15 months, 18 months, 24 months, 30 months, or 36 months subsequent to the previous treatment.

In another embodiment, a subject is administered one or more unit doses of approximately 0.5-50 μg/kg, approximately 0.5-40 μg/kg, approximately 0.5-30 μg/kg, approximately 0.5-20 μg/kg, approximately 0.5-15 μg/kg, approximately 0.5-10 μg/kg, approximately 0.5-5 μg/kg, approximately 1-5 μg/kg, approximately 1-10 μg/kg, approximately 20-40 μg/kg, approximately 20-30 μg/kg, approximately 22-28 μg/kg or approximately 25-26 μg/kg of one or more anti-TCR antibody to prevent, treat or ameliorate one or more symptoms of an autoimmune disorder or T cell malignancy. In another embodiment, a subject is administered one or more unit doses of 200 μg/kg, 178 μg/kg, 180 μg/kg, 128 μg/kg, 100 μg/kg, 95 μg/kg, 90 μg/kg, 85 μg/kg, 80 μg/kg, 75 μg/kg, 70 μg/kg, 65 μg/kg, 60 μg/kg, 55 μg/kg, 50 μg/kg, 4S μg/kg, 40 μg/kg, 35 μg/kg, 30 μg/kg, 26 μg/kg, 25 μg/kg, 20 μg/kg, 15 μg/kg, 13 μg/kg, 10 μg/kg, 6.5 μg/kg, 5 μg/kg, 3.2 μg/kg, 3 μg/kg, 2.5 μg/kg, 2 μg/kg, 1.6 μg/kg, 1.5 μg/kg, 1 μg/kg, 0.5 μg/kg, 0.25 μg/kg, 0.1 μg/kg, or 0.05 μg/kg of one or more anti-TCR antibodies to prevent, treat or ameliorate one or more symptoms of an autoimmune disorder or T-cell malignancy.

In a one embodiment, a subject is administered one or more doses of 200 μg/kg or less, 175 μg/kg or less, 150 μg/kg or less, 128 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, 0.25 μg/kg or less, 0.1 μg/kg or less, or 0.05 μg/kg or less of one or more anti-TCR antibody of the invention to prevent, treat or ameliorate one or more symptoms of an autoimmune disorder or T-cell malignancy.

In particular embodiments, a subject is administered one or more doses of about 5-1200 μg/m², preferably, 51-826 μg/m². In another embodiment, a subject is administered one or more unit doses of 1200 μg/m², 1150 μg/m², 1100 μg/m², 1050 μg/m², 1000 μg/m², 950 μg/m², 900 μg/m², 850 μg/m², 800 μg/m², 750 μg/m², 700 μg/m², 650 μg/m², 600 μg/m², 550 μg/m², 500 μg/m², 450 μg/m², 400 μg/m², 350 μg/m², 300 μg/m², 250 μg/m², 200 μg/m², 150 μg/m², 100 μg/m², 50 μg/m², 40 μg/m², 30 μg/m², 20 μg/m², 15 μg/m², 10 μg/m², or 5 μg/m² of one or more anti-TCR antibodies to prevent, treat, slow the progression of, delay the onset of or ameliorate one or more symptoms of an autoimmune disorder or T-cell malignancy.

In another embodiment, the subject is administered a treatment regimen comprising one or more doses of a prophylactically or therapeutically effective amount of one or more anti-TCR antibodies, wherein the course of treatment is administered over 2 days, 3 days, 4 days, 5 days, 6 days, 7 days, 8 days, 9 days, 10 days, 11 days, 12 days, 13 days or 14 days. In one embodiment, the treatment regimen comprises administering doses of the prophylactically or therapeutically effective amount of one or more anti-TCR antibodies every day, every 2^{nd} day, every 3^{rd} day or every 4^{th} day. In certain embodiments, the treatment regimen comprises administering doses of the prophylactically or therapeutically effective amount of one or more anti-TCR antibodies on Monday, Tuesday, Wednesday, Thursday of a given week and not administering doses of the prophylactically or therapeutically effective amount of one or more anti-TCR antibodies on Friday, Saturday, and Sunday of the same week until 14 doses, 13, doses, 13 doses, 12 doses, 11 doses, 10 doses, 9 doses, or 8 doses have been administered. In certain embodiments the dose administered is the same each day of the regimen. In certain embodiments, a subject is administered a treatment regimen comprising one or more doses of a prophylactically or therapeutically effective amount of one or more anti-TCR antibodies, wherein the prophylactically or therapeutically effective amount is 200 μg/kg/day, 175 μg/kg/day, 150 μg/kg/day, 125 μg/kg/day, 100 μg/kg/day, 95 μg/kg/day, 90 μg/kg/day, 85 μg/kg/day, 80 μg/kg/day, 75 μg/kg/day, 70 μg/kg/day, 65 μg/kg/day, 60 μg/kg/day, 55 μg/kg/day, 50 μg/kg/day, 45 μg/kg/day, 40 μg/kg/day, 35 μg/kg/day, 30 μg/kg/day, 26 μg/kg/day, 25 μg/kg/day, 20 μg/kg/day, 15 μg/kg/day, 13 μg/kg/day, 10 μg/kg/day, 6.5 μg/kg/day, 5 μg/kg/day, 3.2 μg/kg/day, 3 μg/kg/day, 2.5 μg/kg/day, 2 μg/kg/day, 1.6 μg/kg/day, 1.5 μg/kg/day, 1 μg/kg/day, 0.5 μg/kg/day, 0.25 μg/kg/day, 0.1 μg/kg/day, or 0.05 μg/kg/day; and/or wherein the prophylactically or therapeutically effective amount is 1200 μg/m²/day, 1150 μg/m²/day, 1100 μg/m²/day, 1050 μg/m²/day, 1000 μg/m²/day, 950 μg/m²/day, 900 μg/m²/day, 850 μg/m²/day, 800 μg/m²/day, 750 μg/m²/day, 700 μg/m²/day, 650 μg/m²/day, 600 μg/m²/day, 550 μg/m²/day, 500 μg/m²/day, 450 μg/m²/day, 400 μg/m²/day, 350 μg/m²/day, 300 μg/m²/day, 250 μg/m²/day, 200 μg/m²/day, 150 μg/m²/day, 100 μg/m²/day, 50 μg/m²/day, 40 μg/m²/day, 30 μg/m²/day, 20 μg/m²/day, 15 μg/m²/day, 10 μg/m²/day, or 5 μg/m²/day. In another embodiment, the intravenous dose of 1200 μg/m² or less, 1150 μg/m² or less, 1100 μg/m² or less, 1050 μg/m² or less, 1000 μg/m² or less, 950 μg/m² or less, 900 μg/m² or less, 850 μg/m² or less, 800 μg/m² or less, 750 μg/m² or less, 700 μg/m² or less, 650 μg/m² or less, 600 μg/m² or less, 550 μg/m² or less, 500 μg/m² or less, 450 μg/m² or less, 400 μg/m² or less, 350 μg/m² or less, 300 μg/m² or less, 250 μg/m² or less, 200 μg/m² or less, 150 μg/m² or less, 100 μg/m² or less, 50 μg/m² or less, 40 μg/m² or less, 30 μg/m² or less, 20 μg/m² or less, 15 μg/m² or less, 10 μg/m² or less, or 5 μg/m² or less of one or more anti TCR antibodies is administered over about 24 hours, about 22 hours, about 20 hours, about 18 hours, about 16 hours, about 14 hours, about 12 hours, about 10 hours, about 8 hours, about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of an autoimmune disease or T-cell malignancy. The total dosage over the duration of the regimen is preferably a total of less than 9000 μg/m², 8000 μg/m², 7000 μg/m², 6000 μg/m², and may be less than 5000 μg/m², 4000 μg/m², 3000 μg/m², 2000 μg/m², or 1000 μg/m². In specific embodiments, the total dosage administered in the regimen is 100 μg/m² to 200 μg/m², 100 μg/m² to 500 μg/m², 100 μg/m² to 1000 μg/m², or 500 μg/m² to 1000 μg/m².

In preferred embodiments, the dose escalates over the first fourth, first half or first 2/3 of the doses (e.g., over the first 2, 3, 4, 5, or 6 days of a 10, 12, 14, 16, 18 or 20 day regimen of one dose per day) of the treatment regimen until the daily prophylactically or therapeutically effective amount of one or more anti-TCR antibodies is achieved. In certain embodiments, a subject is administered a treatment regimen comprising one or more doses of a prophylactically or therapeutically effective amount of one or more anti-TCR antibodies, wherein the prophylactically or therapeutically effective amount is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg each day; or increased by, e.g., 1 μg/m², 5 μg/m², 10 μg/m², 15 μg/m², 20 μg/m², 30 μg/m², 40 μg/m², 50 μg/m², 60 μg/m², 70 μg/m², 80 μg/m², 90 μg/m², 100 μg/m², 150 μg/m², 200 μg/m², 250 μg/m², 300 μg/m², 350 μg/m², 400 μg/m², 450 μg/m², 500 μg/m², 550 μg/m², 600 μg/m², or 650 μg/m², each day as treatment progresses. In certain embodiments, a subject is administered a treatment regimen comprising one or more doses of a prophylactically or therapeutically effective amount of one or more anti-TCR antibodies, wherein the prophylactically or therapeutically effective amount is increased by a factor of 1.25, a factor of 1.5, a factor of 2, a factor of 2.25, a factor of 2.5, or a factor of 5 until the daily prophylactically or therapeutically effective amount of one or more anti-TCR antibodies is achieved.

In a specific embodiment, a subject is intramuscularly administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more anti-TCR antibodies to prevent, treat or ameliorate one or more symptoms of an autoimmune disorder or T-cell malignancy.

In another embodiment, a subject is subcutaneously administered one or more doses of a 200 μg/kg or less, preferably 175 μg/kg or less, 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more anti-TCR antibodies to prevent, treat or ameliorate one or more symptoms of an autoimmune disorder.

In another embodiment, a subject is intravenously administered one or more doses of a 100 μg/kg or less, preferably 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more anti TCR antibodies to prevent, treat or ameliorate one or more symptoms of an autoimmune disorder or T-cell malignancy. In another embodiment, the intravenous dose of 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of one or more anti-TCR antibodies is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of an autoimmune disorder or T-cell malignancy.

In specific embodiments in which escalating doses are administered for the first days of the dosing regimen, the dose on day 1 of the regimen is 5-100 μg/m²/day, preferably 51 μg/m²/day and escalates to the daily dose as recited immediately above by day 3, 4, 5, 6 or 7. For example, on day 1, the subject is administered a dose of approximately 51 μg/m²/day, on day 2 approximately 103 μg/m²/day, on day 3 approximately 207 μg/m²/day, on day 4 approximately 413 μg/m²/day and on subsequent days of the regimen (e.g., days 5-14) 826 μg/m²/day. In another embodiment, on day 1, the subject is administered a dose of approximately 227 μg/m²/day, on day 2 approximately 459 μg/m²/day, on day 3 and subsequent days, approximately 919 μg/m²/day. In another embodiment, on day 1, the subject is administered a dose of approximately 284 μg/m²/day, on day 2 approximately 574 μg/m²/day, on day 3 and subsequent days, approximately 1148 μg/m²/day.

In other embodiments, the initial dose is ¼, to ½, to equal to the daily dose at the end of the regimen but is administered in portions at intervals of 6, 8, 10 on 12 hours. For example, a 13 ug/kg/day dose is administered in four doses of 3-4 μg/kg at intervals of 6 hours to reduce the level of cytokine release caused by administration of the antibody.

In specific embodiments, to reduce the possibility of cytokine release and other adverse effects, the first 1, 2, 3, or 4 doses or all the doses in the regimen are administered more slowly by intravenous administration. For example, a dose of 51 μg/m²/day may be administered over about 5 minutes, about 15 minutes, about 30 minutes, about 45 minutes, about 1 hour, about 2 hours, about 4 hours, about 6 hours, about 8 hours, about 10 hours, about 12 hours, about 14 hours, about 16 hours, about 18 hours, about 20 hours, and about 22 hours. In certain embodiments, the dose is administered by slow infusion over a period of, e.g., 20 to 24 hours. In specific embodiments, the dose is infused in a pump, preferably increasing the concentration of antibody administered as the infusion progresses.

In other embodiments, a set fraction of the regimen may be administered in escalating doses. For example, for the 51 μg/m²/day to 826 μg/m²/day regimen described above, the fraction may be 1/10, ¼, ⅓, ½, ⅔ or ¾ of the daily doses. Accordingly, when the fraction is 1/10, the daily doses will be 5.1 μg/m² on day 1, 10.3 μg/m² on day 2, 20.7 μg/m² on day 3, 41.3 μg/m² on day 4 and 82.6 μg/m² on days 5 to 14. When the fraction is ¼, the doses will be 12.75 μg/m² on day 1, 25.5 μg/m² on day 2, 51 μg/m² on day 3, 103 μg/m² on day 4, and 207 μg/m² on days 5 to 14. When the fraction is ⅓, the doses will be 17 μg/m² on day 1, 34.3 μg/m² on day 2, 69 μg/m² on day 3, 137.6 μg/m² on day 4, and 275.3 μg/m² on days 5 to 14. When the fraction is ½, the doses will be 25.5 μg/m² on day 1, 51 μg/m² on day 2, 103 μg/m² on day 3, 207 μg/m² on day 4, and 413 μg/m² on days 5 to 14. When the fraction is ⅔, the doses will be 34 μg/m² on day 1, 69 μg/m² on day 2, 137.6 μg/m² on day 3, 275.3 μg/m² on day 4, and 550.1 μg/m² on days 5 to 14. When the fraction is ¾, the doses will be 38.3 μg/m² on day 1, 77.3 μg/m² on day 2, 155.3 μg/m² on day 3, 309.8 μg/m² on day 4, and 620 μg/m² on days 5 to 14.

In specific embodiments, the anti-TCR antibody is not administered by daily doses over a number of days, but is rather administered by infusion in an uninterrupted manner over 4 hours, 6 hours, 8 hours, 10 hours, 12 hours, 15 hours, 18 hours, 20 hours, 24 hours, 30 hours or 36 hours. The infusion may be constant or may start out at a lower dosage for, for example, the first 1, 2, 3, 5, 6, or 8 hours of the infusion and then increase to a higher dosage thereafter. Over the course of the infusion, the patient receives a dose equal to the amount administered in the exemplary regimens set forth above. For example, a dose of approximately 150 μg/m², 200 μg/m², 250 μg/m², 500 μg/m², 750 μg/m², 1000 μg/m², 1500 μg/m², 2000 μg/m², 3000 μg/m², 4000 μg/m², 5000 μg/m², 6000 μg/m², 7000 μg/m², 8000 μg/m², or 9000 μg/m². In particular, the speed and duration of the infusion is designed to minimize the level of free anti-human TCR antibody in the subject after administration. In certain embodiments, the level of free anti-TCR antibody should not exceed 200 ng/ml free antibody. In addition, the infusion is designed to achieve a combined T cell receptor coating and modulation of at least 50%, 60%, 70%, 80%, 90%, 95% or of 100%.

In certain embodiments, the anti-TCR antibody is administered so as to achieve a certain level of combined coating and modulation of T cell receptor complexes on T cells, as determined by methods well known in the art, see, e.g., Example 11 of U.S. patent application publication US 2003/0108548, which is hereby incorporated by reference in its entirety. In specific embodiments, the dosing regimen achieves a combined T cell receptor coating and modulation of at least 50%, 60%, 70%, 80%, 90%, 95% or of 100% with, in specific embodiments, little to no free anti-TCR antibody detected (for example, less than 200 ng/mL of the drug is detected in the blood of the patient).

In preferred embodiments, the anti-TCR antibodies are administered parenterally, for example, intravenously, intramuscularly or subcutaneously, or, alternatively, are administered orally. The anti-TCR antibodies may also be administered as a sustained release formulation.

In a specific embodiment, the administration of one or more doses or a dosage regimen of a prophylactically or therapeutically effective amount of one or more anti-human TCR antibodies does not induce or reduces relative to other immunosuppressive agents one or more of the following unwanted or adverse effects: vital sign abnormalities (fever, tachycardia, bradycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, vasodilatation, an increased risk of opportunistic infection, activation of Epstein Barr Virus, apoptosis of T cells and an increased risk of developing certain types of cancer. In another specific embodiment, the administration of one or more doses of a prophylactically or therapeutically effective amount of one or more anti-TCR antibodies does not induce or reduces relative to other immunosuppressive agents one or more of the following unwanted or adverse effects: vital sign abnormalities (fever, tachycardia, bradycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, vasodilatation, an increased risk of opportunistic infection, Epstein Barr Virus activation, apoptosis of T cells, and an increased risk of developing certain types of cancer.

In accordance with the invention, the dose or dosage regimen comprising a prophylactically or therapeutically effective amount of one or more anti-TCR antibodies for the treatment of an autoimmune disorder may be repeated at 1 month, 2 months, 4 months, 6 months, 8 months, 12 months, 15 months, 18 months or 24 months or longer after the initial or previous dose or dosage regimen comprising a prophylactically or therapeutically effective amount of one or more anti-TCR antibodies. The repeat dose or dosage regimen may be administered as a matter of course, when symptoms associated with said autoimmune disorder recur after an improvement following the initial or previous dose or dosage regimen, or when symptoms associated with said autoimmune disorder do not improve after the initial dose or dosage regimen of anti-TCR antibodies according to methods of the invention. For example, with respect to diabetes, a repeat dose or dosage regimen comprising a prophylactically or therapeutically effective amount of one or more anti-TCR antibodies may be administered to a subject when, for example, the subject's average daily insulin use at 1 month, 2 months, 4 months, 6 months, 8 months, 12 months, 15 months, 18 months or 24 months or longer after initial or previous treatment with anti-TCR antibodies does not decrease by at least 5%, at least 10%, at least 20%, at least 30%, at least 40%, at least 50%, at least 60%, at least 70%, at least 80% or at least 90% compared to pre-treatment levels. Alternatively, with respect to diabetes, a repeat dose or dosage regimen comprising a prophylactically or therapeutically effective amount of one or more anti-TCR antibodies may be administered to a subject when, for example, the subject's HA 1 or HA 1 C levels at 1 month, 2 months, 4 months, 6 months, 8 months, 12 months, 15 months, 18 months or 24 months or longer after initial or previous treatment with anti-TCR antibodies do not decrease by at least 5%, at least 10%, at least 20%, at least 30%, at least 40%, at least 50%, at least 60%, at least 70%, at least 80% or at least 90% compared to pre-treatment levels. In another embodiment, with respect to diabetes, a repeat dose or dosage regimen comprising a prophylactically or therapeutically effective amount of one or more anti-TCR antibodies may be administered to a subject when, for example, the subject's C-peptide response at 1 month, 2 months, 4 months, 6 months, 8 months, 12 months, 15 months, 18 months or 24 months or longer after initial or previous treatment with anti-TCR antibodies decreases by more than 5%, more than 10%, more than 20%, more than 30%, more than 40%, more than 50%, more than 60%, more than 70%, more than 80% or more than 90% compared to pre-treatment levels.

5.10.1 Diabetes

In other embodiments, the anti-TCR antibody is administered chronically to treat, prevent, or slow or delay the onset or progression, or ameliorate one or more symptoms of type 1 diabetes. For example, in certain embodiments, a low dose of the anti-TCR antibody is administered once a month, twice a month, three times per month, once a week or even more frequently either as an alternative to the 6 to 14 day dosage regimen discussed above or after administration of such a regimen to enhance or maintain its therapeutic effect. Such a low dose may be anywhere from 1 μg/m² to 100 μg/m², preferably, approximately 5 μg/m², 10 μg/m², 15 μg/m², 20 μg/m², 25 μg/m², 30 μg/m², 35 μg/m², 40 μg/m², 45 μg/m², or 50 μg/m².

In other embodiments, the subject may be re-dosed at some time subsequent to administration of the anti-TCR antibody dosing regimen, preferably, based upon one or more physiological parameters or may be done as a matter of course. Such redosing may be administered and/or the need for such redosing evaluated 2 months, 4 months, 6 months, 8 months, 9 months, 1 year, 15 months, 18 months, 2 years, 30 months or 3 years after administration of a dosing regimen and may include administering a course of treatment every 6 months, 9 months, 1 year, 15 months, 18 months, 2 years, 30 months or 3 years indefinitely.

In specific embodiments, subjects are administered a subsequent round of anti-human TCR antibody treatment based upon measurements of one or a combination of the following: the CD4/CD8 cell ratio, CD8 cell count, CD4/CD3 inversion, CD4/CD25 cell ratio, CD4/FoxP3 cell ratio, CD4/CD40 cell ratio, CD4/IL-10 cell ratio, and/or a CD4/TGF-β cell ratio.

With respect to the treatment of management of Type 1 diabetes, other parameters for determining whether to administer a subsequent round of treatment include an appearance or an increase in anti-islet cell antibodies, such as GADAs, IA-2 antibodies or anti-insulin antibodies or an appearance or increase in the levels of T cells specific for islet cell antigens. Subsequent doses may be administered if the number of β-cells or β-cell activity or function decreases by 20%, 30%, 40%, 50%, 60%, 70%, 80% or 90% as compared to the β-cell number or activity or function during administration of the preceding round of treatment. β-cell function may be determined by any method know in the art, for example, the C peptide response to MMTT, OGTT, IGTT, or two-phase glucose clamp, or the First Phase Insulin Release (FPIR) test, as discussed above. As used herein, the term “C-peptide” refers to a 31-amino acid peptide cleaved from proinsulin as it is converted to insulin. Proinsulin consists of an A chain, a connecting peptide (C-peptide), and a B chain. After proinsulin is cleaved, C-peptide remains in the secretory granules of beta cells in the pancreas with insulin and is cosecreted with insulin in response to glucose stimulation. C-peptide is thus released from the pancreas in equi-molar amounts with insulin and may be used as a marker of endogenous insulin production.

Other parameters that may be used to determine whether to redose include the HA1 or HA1c levels, the need for administration of exogenous insulin or increase in the dosage of exogenous insulin by more than 0.1 U/kg/day, 0.2 U/kg/day, 0.5 U/kg/day, 0.6 U/kg/day, 1 U/kg/day, or 2 U/kg/day. For example, a subject may be administered a subsequent round of treatment when the C-peptide response or FPIR of the patient to MMTT, OGTT, IGTT or two phase glucose clamp procedure decreases by more than 1%, more than 5%, more than 10%, more than 20%, more than 30%, more than 40% or more than 50% of pretreatment levels. In particular embodiments, subjects are redosed if they have a C-peptide response to MMTT, OGTT, IGTT or two-phase glucose clamp procedure (preferably, MMTT) resulting in an AUC of less than 40 pmol/ml/240 min., less than 50 pmol/ml/240 min, less than 60 pmol/ml/240 min, less than 70 pmol/ml/240 min., less than 80 pmol/ml/240 min., or less than at least 90 pmol/ml/240 min. In specific embodiments, subjects may be redosed they have a FPIR of less than 300 pmol/l, less than 400 pmol/l, less than 500 pmol/l, less than 600 pmol/l, or less than 700 pmol/l, Also for example, a subject may be redosed when the subject's HA1 or HA1C levels increase by at least 5%, at least 10%, at least 20%, at least 30%, at least 40%, at least 50%, at least 60%, at least 70%, at least 80% or at least 90% compared to pre-treatment levels or the absolute levels are greater than 8%, greater than 7.5%, or greater than 7%. In other embodiments, the further doses may be administered based upon appearance of or increase in number (such as an increase by, on average, 1, 2, 3, 4, 5, 8, 10 15, or 20), duration and/or severity of hypoglycemic episodes or of ketoacidosis episodes on a daily, weekly or monthly basis.

In a specific embodiment, anti-human TCR therapy is used in type 1 diabetes patients that have at least 99%, at least 95%, at least 90%, at least 85%, at least 80%, at least 75%, at least 70%, at least 75%, at least 60%, at least 50% residual β-cell function as compared to an individual with no indicators of diabetes or predisposition to diabetes in the same population (i.e, age, sex, race, and general health) and determined by methods described herein or known to one of ordinary skill in the art. In certain embodiments, the predisposition for development of Type I diabetes manifests as an impaired fasting glucose level, i.e., at least one determination of a glucose level of 100-125 mg/dL after fasting (eight hours without food), or is an impaired glucose tolerance in response to a 75 gram oral glucose tolerance test (OGTT), i.e., at least one determination of a 2-hour glucose level of 140-199 mg/dL in response to a 75 OGTT. In other embodiments, the subjects are positive for one or more autoantibodies reactive against islet cell antigens, such as, GAD antibodies, such as GAD 65 and/or GAD 67, IA-2 or anti-insulin antibodies. In other embodiments, the predisposition for development of type 1 diabetes is having a first or second degree relative who is a diagnosed type 1 diabetic. In certain embodiments, the predisposition is positive diagnosis in the patient or in a first or second degree relative according to art accepted criteria of at least one other autoimmune disorder including, but not limited to, thyroid disease, type 1 diabetes, rheumatoid arthritis, systemic lupus erythematosus, multiple endocrine adenopathy, and celiac disease. In some embodiments, the autoimmune disorder is a MHC DR3- and/or a DR4-related autoimmune disease.

In another embodiment, after a course of treatment with an anti-TCR antibody according to the invention, the level of β-cell function of the patient decreases by less than 1%, less than 5%, less than 10%, less than 20%, less than 30%, less than 40% or less than 50% of the pretreatment levels. In yet another embodiment of the invention, after a course of treatment with an anti-TCR antibody according to the invention, the level of β-cell function of the patient is maintained at least 99%, at least 95%, at least 90%, at least 80%, at least 70%, at least 60%, or at least 50% of pretreatment levels for at least 4 months, at least 6 months, at least 9 months, at least 12 months, at least 18 months, at least 24 months, or at least 30 months after the end of treatment.

In certain embodiments, one or more pharmaceutical compositions comprising one or more TCR binding molecules (e.g., one or more anti-TCR antibodies) are administered to a subject having type 1 diabetes, to prevent or slow the reduction in β-cell mass associated with autoimmune diabetes. In some embodiments, after a course of treatment with an anti-TCR antibody according to the invention, the level of β-cell mass of the patient decreases by less than 1%, less than 5%, less than 10%, less than 20%, less than 30%, less than 40%, less than 50%, less than 60%, or less than 70% of the pretreatment levels. In yet another embodiment of the invention, after a course of treatment with an anti-TCR antibody according to the invention, the level of β-cell function of the patient is maintained at least 99%, at least 95%, at least 90%, at least 80%, at least 70%, at least 60%, at least 50%, at least 40%, or at least 30% of pretreatment levels for at least 4 months, at least 6 months, at least 9 months, at least 12 months, at least 18 months, at least 24 months, or at least 30 months after the end of treatment.

In the methods of the invention, the anti-TCR therapy is administered in patients that do not require daily insulin, or that have average insulin requirements of less than 0.05 U/kg/day, less than 0.1 U/kg/day, less than 0.2 U/kg/day, less than 0.4 U/kg/day, less than 0.6 U/kg/day, less than 0.8 U/kg/day, less than 1 U/kg/day, less than 2 U/kg/day, less than 5 U/kg/day, less than 10 U/kg/day or less than 50 U/kg/day. In another embodiment, a patient with an autoimmune diabetes disorder is administered a regimen of doses of a prophylactically or therapeutically effective amount of one or more anti-TCR antibodies to avoid or delay the need to administer insulin, or increase the dose of insulin administered for more than 6 months, 1 year, 18 months, 24 months, 30 months, 36 months, 5 years, 7 years or 10. In other embodiments, in patients who do require exogenous insulin, methods of the invention achieve a reduction in daily insulin requirement by at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, or at least 90% of pretreatment levels. In yet another embodiment of the invention in patients who require exogenous insulin, after a course of treatment with an anti-TCR antibody according to the invention, the reduction of a patient's daily insulin requirements by at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, or at least 90% of pretreatment levels is maintained for at least 4 months, at least 6 months, at least 9 months, at least 12 months, at least 18 months, at least 24 months, or at least 30 months after the course of treatment.

In other embodiments, in patients who do require exogenous insulin, methods of the invention result in an increase in the daily insulin requirement by no more than 1%, no more than 5%, no more than 10%, no more than 15%, no more than 20%, no more than 25%, no more than 30%, no more than 40%, no more than 45%, no more than 50%, no more than 55%, no more than 60%, no more than 65%, no more than 70%, or no more than 75% as compared to pretreatment levels. In yet another embodiment of the invention in patients who require exogenous insulin, after a course of treatment with an anti-TCR antibody according to the invention, the increase in a patient's daily insulin requirements by no more than 1%, no more than 5%, no more than 10%, no more than 15%, no more than 20%, no more than 25%, no more than 30%, no more than 40%, no more than 45%, no more than 50%, no more than 55%, no more than 60%, no more than 65%, no more than 70%, or no more than 75% of pretreatment levels is maintained for at least 4 months, at least 6 months, at least 9 months, at least 12 months, at least 18 months, at least 24 months, or at least 30 months after the course of treatment.

In yet another embodiment, a human subject having type 1 diabetes, or a human identified as having a predisposition to developing type 1 diabetes is administered a course of a prophylactically or therapeutically effective amount of one or more anti-TCR antibodies to preserve the subject's C-peptide response or FPIR to MMTT, OGTT, IGTT or two phase glucose clamp procedure over about 2 weeks, about 1 month, about 2 months, about 4 months, about 5 months, about 6 months, about 7 months, about 8 months, about 9 months, about 10 months, about 11 months, about 12 months, about 15 months, about 18 months, about 21 months or about 24 months after treatment. In preferred embodiments, the patients initially have a C-peptide response to MMTT, OGTT, IGTT, or two-phase glucose clamp procedure (preferably MMTT) resulting in an area under curve (AUC) of at least 80 pmol/ml/240 min., preferably, at least 90 pmol/ml/240 min., more preferably at least 100 pmol/ml/240 min., or even at least 110 pmol/ml/240 min. In preferred embodiments, the patients prior to treatment with an anti-TCR antibody according to the invention have a FPIR of at least 300 pmol/l, at least 350 pmol/l, at least 400 pmol/l, at least 450 pmol/l, at least 500 pmol/l, preferably, at least 550 pmol/l, more preferably at least 600 pmol/l, or even at least 700 pmol/l. In another embodiment of the invention, after a course of treatment with an anti-TCR antibody according to the invention, the C-peptide response or FPIR of the patient to MMTT, OGTT, IGTT, or two-phase glucose clamp procedure decreases by less than 1%, less than 5%, less than 10%, less than 20%, less than 30%, less than 40% or less than 50% of the pretreatment levels. In yet another embodiment of the invention, after a course of treatment with an anti-TCR antibody according to the invention, the C-peptide response or FPIR of the patient to MMTT, OGTT, IGTT or two phase glucose clamp procedure is maintained at least 99%, at least 95%, at least 90%, at least 80%, at least 70%, at least 60%, or at least 50% of pretreatment levels for at least 4 months, at least 6 months, at least 9 months, at least 12 months, at least 18 months, at least 24 months, or at least 30 months after the course of treatment.

In particular embodiments, the invention provides methods of treatment such that a single round of treatment or round of treatment every 6 months, every 9 months, every 12 months, every 15 months, every 18 months, or every 24 months with an anti-TCR antibody (preferably, without any intervening treatment with anti-TCR antibodies), results in a level of HA1 or HA1c that is 7% or less, 6.5% or less, 6% or less, 5.5% or less, or 5% or less 6 months, 9 months, 12 months, 15 months, 18 months, or 24 months after the previous round of treatment or the first round of treatment. In specific embodiments, after a single round of treatment or round of treatment every 6 months, every 9 months, every 12 months, every 15 months, every 18 months, or every 24 months with an anti-TCR antibody according to the methods of the invention (preferably, without any intervening treatment with anti-TCR antibodies), the patients have a C-peptide response to MMTT, OGTT, IGTT or two-phase glucose clamp procedure (preferably, MMTT) resulting in an AUC of at least 40 pmol/ml/240 min., 50 pmol/ml/240 min, 60 pmol/ml/240 min, 70 pmol/ml/240 min., 80 pmol/ml/240 min., preferably, at least 90 pmol/ml/240 min., more preferably at least 100 pmol/ml/240 min., or even at least 110 pmol/ml/240 min, said response determined 6 months, 9 months, 12 months, 15 months, 18 months, or 24 months after the previous round of treatment or after the previous round of treatment. In specific embodiments, after a single round of treatment or round of treatment every 6 months, every 9 months, every 12 months, every 15 months, every 18 months, or every 24 months with an anti-TCR antibody according to the methods of the invention (preferably, without any intervening treatment with anti-TCR antibodies), the patients have a FPIR of at least 300 pmol/l, at least 400 pmol/l, preferably, at least 500 pmol/l, more preferably at least 600 pmol/l, or even at least 700 pmol/l, said FPIR determined at 6 months, 9 months, 12 months, 15 months, 18 months, or 24 months after the previous round of treatment or initial round of treatment.

5.10.2 Multiple Sclerosis (MS)

In another embodiment, with respect to the treatment of MS, pharmaceutical compositions comprising one or more TCR binding molecules (e.g., one or more anti-TCR antibodies) are administered one or more times to prevent or reduce an increase, or slow or reduce an increase in EDSS score associated with MS in a subject. In yet another embodiment, one or more pharmaceutical compositions comprising one or more TCR binding molecules (e.g., one or more anti-TCR antibodies) are administered one or more times to prevent an increase in the frequency, severity and/or duration of attacks associated with MS in a subject. In still other embodiments, one or more pharmaceutical compositions comprising one or more TCR binding molecules (e.g., one or more anti-TCR antibodies) are administered one or ore times to prevent an increase in number and/or total volume of lesions, as detected by, e.g., MRI, associated with MS in a subject. In accordance with these embodiments, the subject's EDSS score and/or a determination of the frequency, duration and/or severity of attacks maybe assessed by a qualified medical practitioner according to methods commonly accepted and well known in the art. In certain embodiments, the subject has benign MS. In other embodiments, the subject has RRMS, SPMS, PRMS, or PPMS. In certain embodiments, one or more pharmaceutical compositions comprising one or more TCR binding molecules (e.g., one or more anti-TCR antibodies) are administered one or more times to reduce the incidence, severity and/or duration of a symptom associated with MS in a subject, wherein said symptoms are described herein or are known in the art. In certain embodiments, symptoms associated with MS include, but are not limited to fatigue, disturbances of vision, disturbances of strength, disturbances of coordination, disturbances of balance, disturbances of bladder/bowel function, weakness or paralysis in one or more extremities, tremor in one or more extremities, muscle spasticity, muscle atropy, dysfunctional movement, numbness or abnormal sensation in any area, tingling, facial pain, extremity pain, loss of vision in one or both eyes, double vision, eye discomfort, uncontrollable rapid eye movements, decreased coordination, loss of balance, decreased ability to control small or intricate movements, walking or gait abnormalities, muscle spasms, dizziness, vertigo, urinary hesitancy, urinary urgency, increased urinary frequency, incontinence, decreased memory, decreased spontaneity, decreased judgment, loss of ability to think abstractly, loss of ability to generalize, depression, decreased attention span, slurred speech, difficulty speaking or understanding speech, fatigue, constipation, hearing loss, and/or positive Babinski's reflex.

In a specific embodiment anti-TCR therapy is used for the treatment of MS in patients that have a disability score according to the Kurtzke Expanded Disability Scale (EDSS) of 1.0, 1.5, 2.0, 2.5, 3.0, 3.5, 4.0, 4.5, 5.0, 5.5, 6.0, 6.5, 7.0, 7.5, 8.0, 8.5, 9.0 or 9.5 as determined by methods described herein or known to one of ordinary skill in the art. In another embodiment, after one or more courses of treatment with an anti-TCR antibody according to the invention the EDSS score of the patient increases by not more than one-half step, not more than one step, not more than one and one-half steps, not more than two steps, not more than two and one-half steps, not more than three steps, not more than three and one-half steps, not more than four steps, not more than four an one-half steps, not more than more than five steps, not more than five and one-half steps, not more than six steps, not more than six and one-half steps, not more than seven steps, not more than seven and one-half steps, not more than eight steps, or not more than eight and one-half steps relative to the pretreatment score.

In yet another embodiment of the invention, after one or more courses of treatment with an anti-TCR. antibody according to the invention the EDSS score of the patient is maintained or increases by not more than one-half step, not more than one step, not more than one and one-half steps, not more than two steps, not more than two and one-half steps, not more than three steps, not more than three and one-half steps, not more than four steps, not more than four an one-half steps, not more than more than five steps, not more than five and one-half steps, not more than six steps, not more than six and one-half steps, not more than seven steps, not more than seven and one-half steps, not more than eight steps, or not more than eight and one-half steps relative to the pretreatment score for at least 4 months, at least 6 months, at least 9 months, at least 12 months, at least 15 months, at least 18 months, at least 24 months, at least 2.5 years or at least 3 years after the end of treatment.

In another embodiment of the invention, after one or more courses of treatment with an anti-TCR antibody according to the invention the EDSS score of the patient is maintained or increases by not more than one-half step, not more than one step, not more than one and one-half steps, not more than two steps, not more than two and one-half steps, not more than three steps, not more than three and one-half steps, not more than four steps, not more than four an one-half steps, not more than more than five steps, not more than five and one-half steps, not more than six steps, not more than six and one-half steps, not more than seven steps, not more than seven and one-half steps, not more than eight steps, or not more than eight and one-half steps relative to the pretreatment score for at least 4 months, at least 6 months, at least 9 months, at least 12 months, at least 15 months, at least 18 months, at least 24 months, at least 2.5 years or at least 3 years after the end of treatment.

In other embodiments, after one or more courses of treatment with an anti-TCR antibody according to the invention the average incidence, frequency, severity or duration of symptoms and/or attacks associated with MS in a patient increases by not more than 2%, 5%, 10%, 15%, 20%, 25%, 30%, 35%, 40%, 45%, 50%, 55%, 60%, b5%, 70% or 75% relative to the pretreatment condition. In yet another embodiment of the invention, after one or more courses of treatment with an anti-TCR antibody according to the invention the average incidence, frequency, severity or duration of symptoms and/or attacks associated with MS in a patient increases by not more than 2%, 5%, 10%, 15%, 20%, 25%, 30%, 35%, 40%, 45%, 50%, 55%, 60%, 65%, 70% or 75% relative to the pretreatment condition for at least 4 months, at least 6 months, at least 9 months, at least 12 months, at least 15 months, at least 18 months, at least 24 months, at least 2.5 years or at least 3 years after the end of treatment.

In another embodiment of the invention, after one or more courses of treatment with an anti-TCR antibody according to the invention the average frequency, severity or duration of symptoms and/or attacks associated with MS in a patient increases by not more than 2%, 5%, 10%, 15%, 20%, 25%, 30%, 35%, 40%, 45%, 50%, 55%, 60%, 65%, 70% or 75% relative to the pretreatment condition for at least 4 months, at least 6 months, at least 9 months, at least 12 months, at least 15 months, at least 18 months, at least 24 months, at least 2.5 years or at least 3 years after the end of treatment.

In another specific embodiment, after one or more courses of treatment with an anti-TCR antibody according to the invention the number and/or total volume of lesions associated with MS as determined by MRI in a patient increases by not more than 2%, 5%, 10%, 15%, 20%, 25%, 30%, 35%, 40%, 45%, 50%, 55%, 60%, 65%, 70% or 75% of the pretreatment condition. In yet another embodiment of the invention, after one or more courses of treatment with an anti-TCR antibody according to the invention the number and/or total volume of lesions associated with MS as determined by MRI in a patient increases by not more than 2%, 5%, 10%, 15%, 20%, 25%, 30%, 35%, 40%, 45%, 50%, 55%, 60%, 65%, 70% or 75% relative to the pretreatment condition for at least 4 months, at least 6 months, at least 9 months, at least 12 months, at least 15 months, at least 18 months, at least 24 months, at least 2.5 years or at least 3 years after the end of treatment.

5.10.3 Psoriasis

In another specific embodiment, after one or more courses of treatment with an anti-TCR antibody according to the invention the Psoriasis Area and Severity Index (PASI) score of a patient having psoriasis decreases by at least 20%, at least 35%, at least 30%, at least 40%, at least 45%, at least 50%, at least 55%, at least 60%, at least 65%, at least 70%, at least 75%, at least 80%, or at least 85% relative to the pretreatment condition. In yet another embodiment of the invention, after one or more courses of treatment with an anti-TCR antibody according to the invention the Psoriasis Area and Severity Index (PASI) score of a patient having psoriasis decreases by at least 20%, at least 35%, at least 30%, at least 40%, at least 45%, at least 50%, at least 55%, at least 60%, at least 65%, at least 70%, at least 75%, at least 80%, or at least 85% relative to the pretreatment condition for at least 4 months, at least 6 months, at least 9 months, at least 12 months, at least 15 months, at least 18 months, at least 24 months, at least 2.5 years or at least 3 years after the end of treatment.

In another embodiment of the invention, after one or more courses of treatment with an anti-TCR antibody according to the invention the Psoriasis Area and Severity Index (PASI) score of a patient diagnosed with psoriasis decreases by at least 20%, at least 35%, at least 30%, at least 40%, at least 45%, at least 50%, at least 55%, at least 60%, at least 65%, at least 70%, at least 75%, at least 80%, or at least 85% relative to the pretreatment condition for at least 4 months, at least 6 months, at least 9 months, at least 12 months, at least 15 months, at least 18 months, at least 24 months, at least 2.5 years or at least 3 years after the end of treatment.

In another specific embodiment, after one or more courses of treatment with an anti-TCR antibody according to the invention the global assessment score of a patient diagnosed with psoriasis improves by at least by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95% relative to pretreatment condition. In yet another embodiment of the invention, after one or more courses of treatment with an anti-TCR antibody according to the invention the global assessment score of a patient diagnosed with psoriasis improves by at least by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95% relative to pretreatment condition for at least 4 months, at least 6 months, at least 9 months, at least 12 months, at least 15 months, at least 18 months, at least 24 months, at least 2.5 years or at least 3 years after the end of treatment.

In another specific embodiment, after one or more courses of treatment with an anti-TCR antibody according to the invention the subject's condition as assessed by any arthritis severity scale known in the art (e.g., RASS) improves by at least 25%, at least 35%, at least 30%, at least 40%, at least 45%, at least 50%, at least 55%, at least 60%, at least 65%, at least 70%, at least 75%, at least 80%, at least 85%, at least 90%, at least 95%, or at least 100% relative to pretreatment condition. In yet another embodiment of the invention, after one or more courses of treatment with an anti-TCR antibody according to the invention the subject's condition as assessed by any arthritis severity scale known in the art (e.g., RASS) improves by at least 25%, at least 35%, at least 30%, at least 40%, at least 45%, at least 50%, at least 55%, at least 60%, at least 65%, at least 70%, at least 75%, at least 80%, at least 85%, at least 90%, at least 95%, or at least 100% relative to pretreatment condition for at least 4 months, at least 6 months, at least 9 months, at least 12 months, at least 15 months, at least 18 months, at least 24 months, at least 2.5 years or at least 3 years after the end of treatment.

In another specific embodiment, after one or more courses of treatment with an anti-TCR antibody according to the invention the absolute number, or proportion, of the subject's autoreactive CTLs as determined by immunospot assay (e.g., ELISPOT) decreases by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to pretreatment condition. In yet another embodiment of the invention, after one or more courses of treatment with an anti-TCR antibody according to the invention the absolute number, or proportion, of the subject's autoreactive CTLs as determined by immunospot assay (e.g., ELISPOT) decreases by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to pretreatment condition for at least 4 months, at least 6 months, at least 9 months, at least 12 months, at least 15 months, at least 18 months, at least 24 months, at least 2.5 years or at least 3 years after the end of treatment.

5.11 Combinatorial Therapy

The present invention provides compositions comprising one or more anti-TCR antibody and one or more prophylactic or therapeutic agents other than anti-TCR antibodies, and methods for immunomodulation in a subject in need thereof, (e.g., preventing, treating, delaying the onset of, slowing the progression of or ameliorating one or more symptoms associated with an autoimmune disorder and/or T cell malignancy) comprising administering to said subject one or more of said compositions. Therapeutic or prophylactic agents include, but are not limited to, peptides, polypeptides, fusion proteins, nucleic acid molecules, small molecules, mimetic agents, synthetic drugs, inorganic molecules, and organic molecules. Any agent which is known to be useful, or which has been used or is currently being used for the prevention, treatment or amelioration of one or more symptoms associated with an autoimmune disorder or T cell malignancy, can be used in combination with an anti-TCR antibody in accordance with the invention described herein. For example, agents commonly used in the treatment of one or more autoimmune disorders include, but are not limited to, antibody fragments, GLP-1 analogs or derivatives, GLP-1 agonists (e.g. exendin-4; exentatide), amylin analogs or derivatives, insulin, dermatological agents for rashes and swellings (e.g., phototherapy (i.e., ultraviolet B radiation), photochemotherapy (e.g., PUVA) and topical agents such as emollients, salicylic acid, coal tar, topical steroids, topical corticosteroids, topical vitamin D3 analogs (e.g., calcipotriene), tazarotene, and topical retinoids), anti-inflammatory agents (e.g., corticosteroids (e.g., prednisone and hydrocortisone}, glucocorticoids, steroids, non-steriodal anti-inflammatory drugs (e.g., aspirin, ibuprofen, diclofenac, and COX-2 inhibitors), beta-agonists, anticholinergic agents and methyl xanthines}, immunomodulatory agents (e.g., small organic molecules, a T cell receptor modulators, cytokine receptor modulators, T cell depleting agents, cytokine antagonists, monokine antagonists, lymphocyte inhibitors, or anti-cancer agents), gold injections, sulphasalazine, penicillamine, anti-angiogenic agents (e.g., angiostatin, TNF-a antagonists (e.g., anti-TNFα antibodies), and endostatin), dapsone, psoralens (e.g., methoxalen and trioxsalen), anti-malarial agents (e.g., hydroxychloroquine), anti-viral agents, and antibiotics (e.g., erythomycin and penicillin). Additional immunomodulatory agent other than the anti-TCR antibody of the invention well-known to one of skill in the art may also be used in the methods and compositions of the invention. Such immunomodulatory agents can affect one or more or all aspects of the immune response in a subject and may or may not affect the same aspect as that of the antibodies of the invention. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In certain embodiments of the invention, an immunomodulatory agent modulates one aspect of the immune response separate from that modulated by the anti-TCR antibody of the invention. In other embodiments of the invention, an immunomodulatory agent modulates one aspect of the immune response which is the same as that modulated by the anti-TCR antibody of the invention. In other embodiments, an immunomodulatory agent modulates more than one aspect of the immune response which may or may not be the same as that (those) modulated by the anti-TCR antibodies of the invention. In a preferred embodiment of the invention, the administration of an immunomodulatory agent to a subject inhibits or reduces one or more aspects of the subject's immune response capabilities. In a specific embodiment of the invention, the immunomodulatory agent inhibits or suppresses the immune response in a subject. In accordance with the invention, an immunomodulatory agent is not an anti-TCR antibody. In certain embodiments, an immunomodulatory agent is not an anti-inflammatory agent. In other embodiments, an immunomodulatory agent is not a TCR binding molecule.

The TCR antibody of the invention and/or the immunomodulatory agent may be selected to interfere with the interactions between the T helper subsets (TH1 or TH2) and B cells to inhibit neutralizing antibody formation. The TCR antibody of the invention and/or the immunomodulatory agent may be selected to inhibit the interaction between TH1 cells and CTLs to reduce the occurrence of CTL-mediated killing. The TCR antibody of the invention and/or the immunomodulatory agent may be selected to alter (e.g., inhibit or suppress) the proliferation, differentiation, activity and/or function of the CD4⁺ and/or CD8⁺ T cells.

In specific embodiments, the anti-TCR antibody is co-administered with a cytokine antagonist. In other embodiments, the anti-TCR antibody is co-administered with an anti-IL-2 antibody, such as, for example, daclizumab, basiliximab or MT204 (Micromet) or other IL-2 inhibitor, such as but not limited to rapamycin, cyclosporine, or tacrolimus.

In a preferred embodiment, proteins, polypeptides or peptides (including antibodies) that are utilized as prophylactic, therapeutic or immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

In accordance with the invention, one or more prophylactic, therapeutic or immunomodulatory agents are administered to a subject with an inflammatory or autoimmune disease prior to, subsequent to, or concomitantly with the therapeutic and/or prophylactic agents of the invention. Preferably, one or more prophylactic, therapeutic or immunomodulatory agents are administered to a subject with an inflammatory or autoimmune disease to reduce or inhibit one or more symptoms of the disease or aspects of the immune response as necessary. Any technique well-known to one skilled in the art can be used to measure one or more aspects of the immune response in a particular subject, and thereby determine when it is necessary to administer an immunomodulatory agent to said subject.

In one embodiment, the present invention provides a method for preventing, treating, managing or ameliorating one or more symptoms associated with diabetes, said method comprising administering to said subject a prophylactically or therapeutically effective amount of one or more anti-TCR antibodies and a prophylactically or therapeutically effective amount of insulin. In one embodiment, the present invention provides a method for preventing, treating, managing or ameliorating one or more symptoms associated with diabetes, said method comprising administering to said subject a prophylactically or therapeutically effective amount of one or more anti-TCR antibodies and a prophylactically or therapeutically effective amount of GLP 1 or GLP 1 analog. In one embodiment, the present invention provides a method for preventing, treating, managing or ameliorating one or more symptoms associated with diabetes, said method comprising administering to said subject a prophylactically or therapeutically effective amount of one or more anti-TCR antibodies and a prophylactically or therapeutically effective amount of exendin-4 or analog thereof. In one embodiment, the present invention provides a method for preventing, treating, managing or ameliorating one or more symptoms associated with diabetes, said method comprising administering to said subject a prophylactically or therapeutically effective amount of one or more anti-TCR antibodies and a prophylactically or therapeutically effective amount of amylin or an analog thereof. In another embodiment, the present invention provides a method for preventing, treating, managing or ameliorating one or more symptoms associated with diabetes, said method comprising administering to said subject a prophylactically or therapeutically effective amount of one or more anti-TCR antibodies and a prophylactically or therapeutically effective amount of insulin. In another embodiment, the present invention provides a method for preventing, treating, managing or ameliorating one or more symptoms associated with psoriasis, said method comprising administering to said subject a prophylactically or therapeutically effective amount of one or more anti-TCR antibodies and a prophylactically or therapeutically effective amount of methotrexate.

In a preferred embodiment, one or more prophylactic, therapeutic or immunomodulatory agents are administered to a subject with an autoimmune disorder so as to transiently reduce or inhibit one or more aspects of the disease or of the immune response. Such a transient inhibition or reduction of one or more aspects of the disease or of the immune system can last for hours, days, weeks, or months. Preferably, the transient inhibition or reduction in one or more aspects of the disease or of the immune response last for a few hours (e.g., 2 hours, 4 hours, 6 hours, 8 hours, 12 hours, 14 hours, 16 hours, 18 hours, 24 hours, 36 hours, or 48 hours), a few days (e.g., 3 days, 4 days, 5 days, 6 days, 7 days, or 14 days), or a few weeks (e.g., 3 weeks, 4 weeks, 5 weeks or 6 weeks). The transient reduction or inhibition of one or more aspects of the disease or of the immune response enhances the prophylactic and/or therapeutic capabilities of an anti-TCR antibody.

In accordance with the invention, one or more prophylactic, therapeutic or immunomodulatory agents are administered to a subject with type 1 diabetes, multiple sclerosis or psoriasis (or other autoimmune disorder), or a predisposition thereto, prior to, subsequent to, or concomitantly with the therapeutic and/or prophylactic agents of the invention. Such methods may be employed to treat, prevent, delay the onset of, slow the progression of or ameliorate one or more symptoms of type 1 diabetes, multiple sclerosis or psoriasis, or other autoimmune disorder.

Nucleic acid molecules encoding proteins, polypeptides, or peptides with prophylactic, therapeutic or immunomodulatory activity or proteins, polypeptides, or peptides with prophylactic, therapeutic or immunomodulatory activity can be administered to a subject with an autoimmune disorder in accordance with the methods of the invention. Further, nucleic acid molecules encoding derivatives, analogs, fragments or variants of proteins, polypeptides, or peptides with prophylactic, therapeutic or immunomodulatory activity, or derivatives, analogs, fragments or variants of proteins, polypeptides, or peptides with prophylactic, therapeutic or immunomodulatory activity can be administered to a subject in accordance with the methods of the invention. Preferably, such derivatives, analogs, variants and fragments retain the prophylactic, therapeutic or immunomodulatory activity of the full-length wild-type protein, polypeptide, or peptide.

Proteins, polypeptides, or peptides that can be used as prophylactic, therapeutic or immunomodulatory agents can be produced by any technique well-known in the art or described herein. See, e.g., Chapter 16 Ausubel et al. (eds.), 1999, Short Protocols in Molecular Biology, Fourth Edition, John Wiley & Sons, NY, which describes methods of producing proteins, polypeptides, or peptides, and which is incorporated herein by reference in its entirety. Antibodies which can be used as prophylactic, therapeutic or immunomodulatory agents can be produced by, e.g., methods described in U.S. Pat. No. 6,245,527 and in Harlow and Lane Antibodies: A Laboratory Manual, Cold Spring Harbor Laboratory Press, Cold Spring Harbor, N.Y., 1988, which are incorporated herein by reference in their entirety. Preferably, agents that are commercially available and known to function as prophylactic, therapeutic or immunomodulatory agents are used in the compositions and methods of the invention. The prophylactic, therapeutic or immunomodulatory activity of an agent can be determined in vitro and/or in vivo by any technique well-known to one skilled in the art, including, e.g., by CTL assays, proliferation assays, and immunoassays (e.g. ELISAs) for the expression of particular proteins such as co-stimulatory molecules and cytokines

The combination of one or more anti-TCR antibodies and one or more prophylactic or therapeutic agents other than anti-TCR antibodies produces a better prophylactic or therapeutic effect in a subject than either treatment alone. In certain embodiments, the combination of an anti-TCR antibody and a prophylactic or therapeutic agent other than an anti-TCR antibody achieves a 20%, 25%, 30%, 35%, 40%, 45%, 50%, 55%, 60%, 65%, 70%, 75%, 80%, 85%, 90%, 95% or 98% better prophylactic or therapeutic effect in a subject with the autoimmune disorder or T cell malignancy, or predisposition thereto, than either treatment alone. In particular embodiments, the combination of one or more anti-TCR antibodies and a prophylactic or therapeutic agent other than an anti-TCR antibody achieves a 20%, preferably a 25%, 30%, 35%, 40%, 45%, 50%, 55%, 60%, 65%, 70%, 75%, 80%, 85%, 90%, 95% or 98% greater reduction in the inflammation of a particular organ, tissue or joint in a subject with an inflammatory disorder or an autoimmune disorder which is associated with inflammation than either treatment alone. In other embodiments, the combination of one or more anti-TCR antibodies and one or more prophylactic or therapeutic agents other than anti-TCR antibodies has an a more than additive effect or synergistic effect in a subject with an autoimmune or inflammatory disorder.

The combination therapies of the invention enable lower dosages of anti-TCR antibodies and/or less frequent administration of anti-TCR antibodies to a subject with an autoimmune disorder or T cell malignancy to achieve a prophylactic or therapeutic effect. The combination therapies of the invention enable lower dosages of the prophylactic or therapeutic agents utilized in conjunction with anti-TCR antibodies and/or less frequent administration of such prophylactic or therapeutic agents to achieve a prophylactic or therapeutic effect.

The prophylactic or therapeutic agents of the combination therapies of the present invention can be administered concomitantly, concurrently or sequentially. The prophylactic or therapeutic agents of the combination therapies of the present invention can also be cyclically administered. Cycling therapy involves the administration of a first prophylactic or therapeutic agent for a period of time, followed by the administration of a second prophylactic or therapeutic agent for a period of time and repeating this sequential administration, i.e., the cycle, in order to reduce the development of resistance to one of the agents, to avoid or reduce the side effects of one of the agents, and/or to improve the efficacy of the treatment.

5.12 T-Cell Malignancies

The invention encompasses methods of using a anti-TCR antibody for the treatment of T-cell malignancies, e.g., as a targeting molecule. Tumors of T-cell origin and other cells involved in T-cell development have been identified. T-cell lymphoproliferative disorders include thymic and post-thymic malignancies. T-cell neoplasms include tumors of lymphoid progenitor cells, thymic stromal or epithelial cells, thymocytes, T-cells, natural killer (“NK”) cells, or antigen-presenting cells. T-cell malignancies include acute lymphoblastic leukemias, lymphomas, thymomas, acute lymphoblastic leukemias, Hodgkin's and non-Hodgkin's disease. Lymphomas are categorized by how the T-cells are affected. T-cell lymphomas include, for example, lymphoblastic lymphoma, anaplastic large cell lymphoma, peripheral T-cell lymphomas, angioimmunoblastic lymphoma, angiocentric lymphoma (nasal T-cell lymphoma), intestinal T-cell lymphoma, and adult T-cell lymphoma/leukemia, some of which are discussed below. Other T-cell lymphoproliferative disorders include T-cell and NK-cell neoplasms, nodular lymphocyte predominant Hodgkin lymphoma, classical Hodgkin lymphoma, nodular sclerosis, lymphocyte-rich classical Hodgkin lymphoma, mixed cellularity classical Hodgkin lymphoma, lymphocyte-depleted classical Hodgkin lymphoma, precursor T-cell neoplasms, precursor T lymphoblastic leukemia lymphoma, blastic NK cell lymphoma, mature T-cell and NK T-cell prolymphocytic leukemia cell neoplasms, T-cell large granular lymphocytic leukemia, aggressive NK cell leukemia, adult T-cell leukemia/lymphoma, extranodal NK/Tcell lymphoma (nasal type), enteropathy-type T-cell lymphoma, hepatosplenic T-cell lymphoma, primary cutaneous anaplastic large cell lymphoma, peripheral T-cell lymphoma, unspecified angioimmunoblastic T-cell lymphoma, and anaplastic large cell lymphoma.

5.12.1 Lymphoblastic Lymphoma

Lymphoblastic lymphoma is an aggressive mostly T-cell lymphoma which occurs mainly in children and adolescents, where it accounts for about half of childhood lymphomas. About two-thirds of the patients are males. A second peak is seen again in patients over 40 years of age. The distinction between lymphoblastic lymphoma and acute lymphoblastic leukemia is, in part, arbitrarily, based on the degree of marrow involvement. The chief biologic difference is that lymphoblastic leukemias are predominantly B-cell diseases, unlike the extra-medullary, mostly T-cell lymphoblastic lymphomas.

5.12.2 T-Cell Prolymphocytic Leukemia (“T-PLL”)

T-cell prolymphocytic leukemia is a rare aggressive post-thymic malignancy with distinctive clinical and morphological and cytogenetic features (See review Matutes E. et al., 1991 Blood, 78: 3269-74). T-PLL is resistant to chemotherapy and has a poor median survival (7.5 months). Although some patients may initially present with indolent disease they eventually progress and the outcome is then similar. New therapeutic approaches are needed to improve the outcome of this fatal disease.

5.12.3 Adult T-Cell Leukemia/Lymphoma (“ATL”)

Adult T-cell leukemia (“ATL”) is one of the T cell malignant neoplasms associated with human T cell leukemia virus type-I (HTLV-I). It is an aggressive fatal malignancy of mature CD4+ lymphocytes (See review Hatta et al., 2002, Leukemia, 16: 1069-85; Yamada Y. 1983, Blood, 61: 192-9). ATL is prevalent in Southern Japan and the Caribbean basin and occurs sporadically in Africa, Latin America, the Middle East, and the United States. ATL has a poor prognosis due to an intrinsic resistance of leukemic cells to conventional chemotherapy.

ATL has been classified into four main subtypes. In the relatively smoldering and chronic forms, the median survival is 2 years or more. In the acute or lymphomatous forms, the media survival is 13 months. Hematopoeitic stem cell transplantation and chemotherapy has been used for the treatment of ATL.

5.12.4 Anaplastic Large Cell Lymphoma

Anaplastic large cell lymphoma (“ALCL”) can be systemic in children or young adults or cutaneous (in/on the skin) Disease limited to the skin is quite slow growing (indolent) and remains localized to the skin with many examples of spontaneous remission—this so-called “classic” ALCL is most common in children and adolescents and has a high frequency of gene translocation t(2; 5). Primary cutaneous ALCL tends to occur more in adults and lacks the translocation. Most cases are T-cell or cell type unknown (null). The systemic form of ALCL may involve lymph nodes and extranodal sites. Chemotherapy has been used to treat the systemic form of ALCL.

5.13 Pharmaceutical Compositions

The present invention provides compositions for the treatment, prophylaxis, and amelioration of one or more symptoms associated with an autoimmune disorder or T cell malignancy. In a specific embodiment, a composition comprises one or more anti-TCR antibodies. In another embodiment, a composition comprises one or more nucleic acid molecules encoding the heavy and light chains of one or more anti-TCR antibodies.

In a specific embodiment, a composition comprises an anti-TCR antibody, wherein said anti-TCR antibody is a human or humanized monoclonal antibody, which, in certain embodiments, is modified to reduce binding of the Fc domain to Fc receptors and, thereby, reduce toxicity of the antibody. In another embodiment, a composition comprises an anti-TCR antibody, wherein said anti-TCR antibody is a human or humanized monoclonal antibody, which is modified to increase binding of the Fc domain to Fc receptors and, thereby, increase the cytotoxicity of the antibody for cells expressing the TCR. In yet another preferred embodiment, a composition comprises humanized BMA 031, an analog, derivative, fragment thereof that immunospecifically binds to the extracellular constant domain of the TCR α chain. In other embodiments, the composition of the invention comprises humanized 3H2906, IP26, T10B9, WT31, G-11, H-197, H-41, A-20, FL-189, H-20, H-250, C-17, H57, KJ1-26, KJ12-98, H28-710, UC3-10A6, GL3, 3H2928, 5K24, 3A10, UC7-13D5, or an analog, derivative or antigen binding fragment thereof.

In a preferred embodiment, a composition of the invention is a pharmaceutical composition. Such compositions comprise a prophylactically or therapeutically effective amount of one or more anti-TCR antibodies, or antigen binding fragments thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like (See, for example, Handbook of Pharmaceutical Excipients, Arthur H. Kibbe (ed., 2000, which is incorporated by reference herein in its entirety), Am. Pharmaceutical Association, Washington, D.C. The composition, if desired, can also contain minor amounts of wetting or emulsifying agents, or pH buffering agents. These compositions can take the form of solutions, suspensions, emulsion, tablets, pills, capsules, powders, sustained release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as silastic membranes, or fibers. Preferably, when administering an anti-human TCR antibody, care must be taken to use materials to which the anti-TCR antibody does not absorb.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 3 17-327; see generally ibid.).

In yet another embodiment, the composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g., U.S. Pat. No. 4,526,938; PCT publication WO 91/05548; PCT publication WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of, e.g., an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

If the compositions of the invention are to be administered orally, the compositions can be formulated orally in the form of, e.g., tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In specific embodiments, the invention provides dosage forms that permit administration of the anti-TCR antibodies continuously over a period of hours or days (e.g., associated with a pump or other device for such delivery), for example, over a period of 1 hour, 2 hours, 3 hours, 4 hours, 6 hours, 8 hours, 10 hours, 12 hours. 16 hours, 20 hours, 24 hours, 30 hours, 36 hours, 4 days, 5 days, 7 days, 10 days or 14 days. In other specific embodiments, the invention provides dosage forms that permit administration of a continuously increasing dose, for example, increasing from 51 μg/m²/day to 826 μg/m²/day over a period of 24 hours, 30 hours, 36 hours, 4 days, 5 days, 7 days, 10 days or 14 days.

The compositions of the invention may also be formulated in rectal compositions such as suppositories or retention enemas, e.g., containing conventional suppository bases such as cocoa butter or other glycerides.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the invention provides that one or more anti-TCR antibodies (or antigen binding fragments thereof), or pharmaceutical compositions of the invention is packaged in a hermetically sealed container such as an ampoule or sachette indicating the quantity of the agent. In one embodiment, one or more of the anti-TCR antibodies, or pharmaceutical compositions of the invention is supplied as a dry sterilized lyophilized powder or water free concentrate in a hermetically sealed container and can be reconstituted, e.g., with water or saline to the appropriate concentration for administration to a subject. Preferably, one or more of the anti-TCR antibodies, or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the invention should be stored at between 2 and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the anti-TCR antibodies, or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

In a preferred embodiment, the invention provides that the composition of the invention is packaged in a hermetically sealed container such as an ampoule or sachette indicating the quantity of anti-TCR antibody.

The compositions may, if desired, be presented in a pack or dispenser device that may contain one or more unit dosage forms containing the active ingredient. The pack may for example comprise metal or plastic foil, such as a blister pack.

Generally, the ingredients of the compositions of the invention are derived from a subject that is the same species origin or species reactivity as recipient of such compositions. Thus, in a preferred embodiment, human or humanized antibodies are administered to a human patient for therapy or prophylaxis.

The amount of the composition of the invention which will be effective in the treatment, prevention or amelioration of one or more symptoms associated with an autoimmune diabetes disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

5.14 Polynucleotides Encoding Antibodies

The invention provides polynucleotides comprising a nucleotide sequence encoding an antibody that immunospecifically binds to a TCR polypeptide. The invention also encompasses polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. The nucleotide sequence of antibodies immunospecific for a TCR polypeptide can be obtained, e.g., from the literature or a database such as GenBank. Since the amino acid sequences of, e.g., BMA 031 is known, nucleotide sequences encoding these antibodies can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a TCR polypeptide. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

5.15 Recombinant Expression of Molecules of the Invention

Once a nucleic acid sequence encoding molecules of the invention (i.e., antibodies) has been obtained, the vector for the production of the molecules may be produced by recombinant DNA technology using techniques well known in the art. Methods which are well known to those skilled in the art can be used to construct expression vectors containing the coding sequences for the molecules of the invention and appropriate transcriptional and translational control signals. These methods include, for example, in vitro recombinant DNA techniques, synthetic techniques, and in vivo genetic recombination. (See, for example, the techniques described in Sambrook et al., 1990, Molecular Cloning, A Laboratory Manual, 2d Ed., Cold Spring Harbor Laboratory, Cold Spring Harbor, N.Y. and Ausubel et al. eds., 1998, Current Protocols in Molecular Biology, John Wiley & Sons, NY).

An expression vector comprising the nucleotide sequence of a molecule identified by the methods of the invention (i.e., an antibody) can be transferred to a host cell by conventional techniques (e.g., electroporation, liposomal transfection, and calcium phosphate precipitation) and the transfected cells are then cultured by conventional techniques to produce the molecules of the invention. In specific embodiments, the expression of the molecules of the invention is regulated by a constitutive, an inducible or a tissue, specific promoter. In specific embodiments the expression vector is pMGX1303 (FIG. 3).

The host cells used to express the molecules identified by the methods of the invention may be either bacterial cells such as Escherichia coli, or, preferably, eukaryotic cells, especially for the expression of whole recombinant immunoglobulin molecule. In particular, mammalian cells such as Chinese hamster ovary cells (CHO), in conjunction with a vector such as the major intermediate early gene promoter element from human cytomegalovirus is an effective expression system for immunoglobulins (Foecking et al., 1998, Gene 45:101; Cockett et al., 1990, Bio/Technology 8:2).

A variety of host-expression vector systems may be utilized to express the molecules identified by the methods of the invention. Such host-expression systems represent vehicles by which the coding sequences of the molecules of the invention may be produced and subsequently purified, but also represent cells which may, when transformed or transfected with the appropriate nucleotide coding sequences, express the molecules of the invention in situ. These include, but are not limited to, microorganisms such as bacteria (e.g., E. coli and B. subtilis) transformed with recombinant bacteriophage DNA, plasmid DNA or cosmid DNA expression vectors containing coding sequences for the molecules identified by the methods of the invention; yeast (e.g., Saccharomyces Pichia) transformed with recombinant yeast expression vectors containing sequences encoding the molecules identified by the methods of the invention; insect cell systems infected with recombinant virus expression vectors (e.g., baculovirus) containing the sequences encoding the molecules identified by the methods of the invention; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the molecules identified by the methods of the invention; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the molecule being expressed. For example, when a large quantity of such a protein is to be produced, for the generation of pharmaceutical compositions of an antibody, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free gluta-thione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express an antibody of the invention may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibodies of the invention. Such engineered cell lines may be particularly useful in screening and evaluation of compounds that interact directly or indirectly with the antibodies of the invention.

A number of selection systems may be used, including but not limited to the herpes simplex virus thymidine kinase (Wigler et al., 1977, Cell 11: 223), hypoxanthine-guanine phosphoribosyltransferase (Szybalska & Szybalski, 1992, Proc. Natl. Acad. Sci. USA 48: 202), and adenine phosphoribosyltransferase (Lowy et al., 1980, Cell 22: 817) genes can be employed in tk-, hgprt- or aprt-cells, respectively. Also, antimetabolite resistance can be used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); neo, which confers resistance to the aminoglycoside G-418 Clinical Pharmacy 12: 488-505; Wu and Wu, 1991, 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1; and hygro, which confers resistance to hygromycin (Santerre et al., 1984, Gene 30:147).

The expression levels of an antibody of the invention can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing an antibody is amplifiable, increase in the level of inhibitor present in culture of host cell will increase the number of copies of the marker gene. Since the amplified region is associated with the nucleotide sequence of the antibody, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once a molecule of the invention (i.e., antibodies) has been recombinantly expressed, it may be purified by any method known in the art for purification of polypeptides or antibodies, for example, by chromatography (e.g., ion exchange, affinity, particularly by affinity for the specific antigen after Protein A, and sizing column chromatography), centrifugation, differential solubility, or by any other standard technique for the purification of polypeptides or antibodies.

### 6. EXAMPLES

6.1 Anti-TCR Monoclonal Antibody Therapy in a Murine Model of Autoimmune Disease, Collagen-Induced Arthritis

Therapeutic efficacy of anti-TCR antibody for the treatment of autoimmune disorders was assessed in the murine collagen induced arthritis (CIA) model. CIA is initiated by inoculation of mice with type II collagen, is mediated a CD4⁺ response, and is characterized by the presence and expansion of multiple lineages of TCR positive cells (including CD4⁺, CD4⁻, and CD8⁻) within the arthritic joints and peripheral lymphoid tissue of CIA-induced mice. Administration of anti-TCR antibody to CIA-induced mice reversed disease symptoms or prevented disease progression both during treatment and for a prolonged period after the end of treatment, demonstrating the potential of anti-TCR antibody as a therapeutic.

Methods: CIA was induced in male and female mice by intradermal injection of type II collagen. All mice were boosted with additional injections of collagen at 21 and 42 days after initial injection. Mice were subsequently inspected for signs of arthritis as determined by paw swelling. Disease progression was monitored by measuring maw thickness using a caliper.

Mice were randomized into treatment groups and injected i.p. with 50 μg of H57 (a hamster monoclonal IgG2a anti-murine TCR antibody; available from the ATCC® under ATCC® number HB-218™), H57 AA, or, as control, 2C11 AA (a murine monoclonal IgG2a anti-CD3 antibody) on each of days 14-18 post initial injection of collagen. The “AA” after H57 or 2C 11 refers to an antibody comprising an Fc domain having the “ala-ala” mutation. The ala-ala mutation abrogates binding of the Fc region of an antibody to all Fc receptors and comprises an Fc region having an alanine at those residues corresponding to positions 234 and 235 according to Kabat.

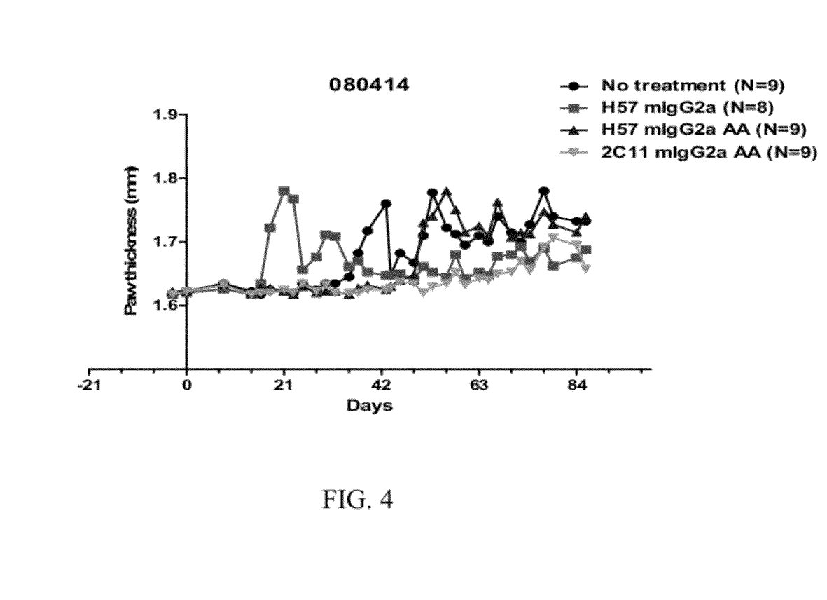

Results: Treatment with anti-TCR antibody resulted in a suppression of disease progression or amelioration of disease symptoms both immediately after treatment and for a period of at least 25 days post treatment (FIG. 4). However, after the second booster of collagen at day 42, mice having received treatments of anti-TCR antibody having the ala-ala mutation exhibited rapid disease progression to levels comparable to those in untreated controls within 3 days. Mice receiving anti-TCR antibody having a wild-type Fc region showed an amelioration of disease symptoms over the study period, which symptoms eventually fell to treatment control levels by study end.

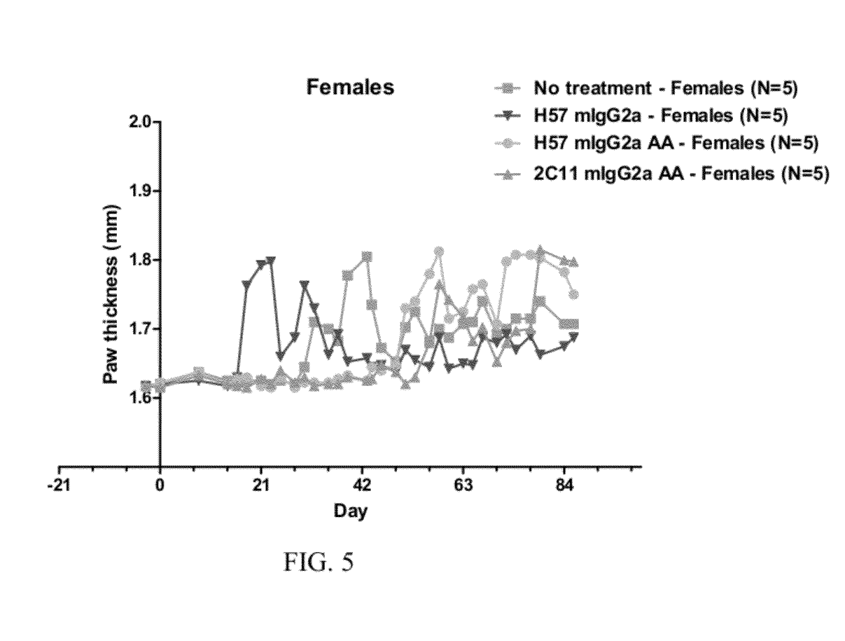

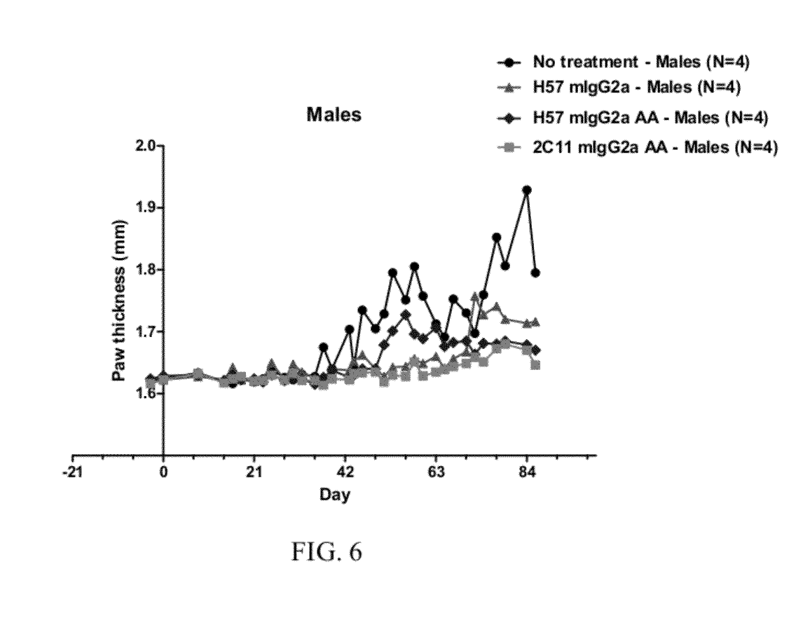

Investigation of the initial spike in disease state observed in the wild-type Fc domain study group revealed the peak to be a result of rapid disease progression in the female mice, of this treatment group (FIG. 5), which progression was not observed in the male counterparts (FIG. 6). However, despite this initial progression, in female mice, the anti-TCR comprising the wild-type Fc did effect an amelioration of disease symptoms such that by study end paw thickness was significantly reduced relative to its peak at day 21. In male mice receiving anti-TCR antibody having a wild type Fc region (the H57 group), the antibody therapy controlled disease progression until at least day 70 post initiation (FIG. 6).

Female and male mice also exhibited differences in response to treatment with “ala-ala” antibody (H57 AA). In female mice, the ala-ala antibody effectively controlled disease progression until the second booster of collagen. After the second booster, both the ala-ala treatment and control 2C 11 female groups exhibited rapid disease progression to levels above untreated control. Although disease progression after the second booster was also observed in the male ala-ala group, the symptoms thereafter decreased to levels matching those of the treatment control group by study end. The results suggest that therapeutic efficacies of the molecules of the invention may differ between genders, leading to gender-specific treatment embodiments.

6.2 Anti-TCR Monoclonal Antibody Therapy in a Murine Model of Autoimmune Disease, Experimental Allergic Encephalomyelitis

Therapeutic efficacy of anti-TCR antibody for the treatment of autoimmune disorders was assessed in the murine experimental allergic encephalomyelitis model. Experimental autoimmune encephalomyelitis (EAE) is an inflammatory demyelinating disease of the central nervous system widely studied as an animal model of multiple sclerosis. EAE can be induced by inoculation of animals with homogenized CNS tissue or highly purified myelin proteins such as myelin basic protein (MBP) or proteolipid protein (PLP). Administration of anti-TCR antibody to EAE-induced mice exhibiting established disease reversed disease symptoms and prevented disease progression, during treatment and for a prolonged period after the end of treatment.

Methods: EAE was induced in mice by subcutaneous injection of 50 μg of PLP₁₃₉₋₁₅₁ emulsified in Complete Freund's Adjuvant, H37Ra (prepared from the avirulent Mycobacterium tuberculosis strain H37Ra) followed by intraperitoneal (IP) injection of 200 ng pertussis toxin. Mice were monitored for development of disease and assessed for disease progression according to standard EAE scoring as known in the art.

Mice were randomized into treatment groups and injected i.v. with 50 μg of H57 AA on each of days 6-10 (n=7) or on each of days 14-17 (n=7) post initial injection of PLP, or were given no treatment (n=6) as control.

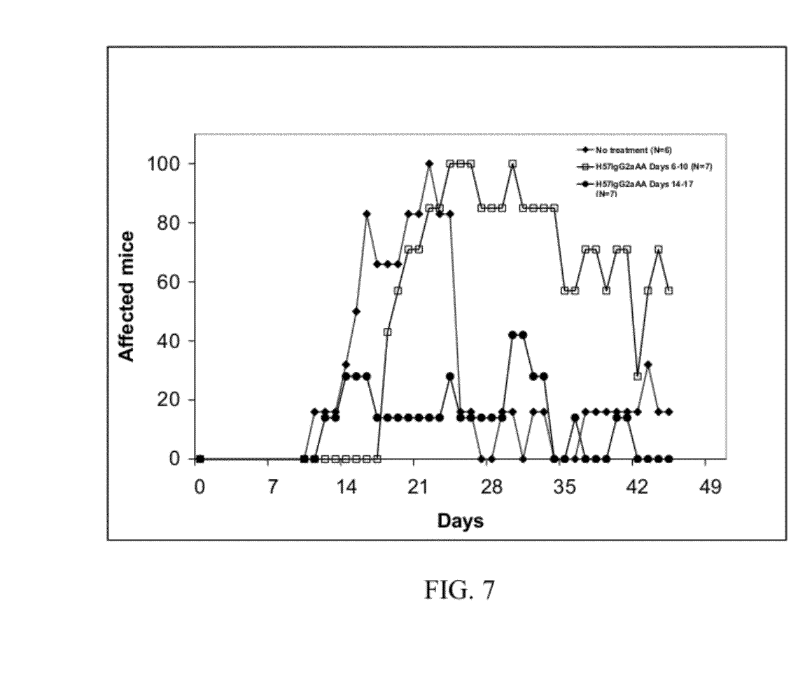

Results: Treatment with anti-TCR antibody halted disease progression and resulted in suppression of disease symptoms both immediately after treatment and for the remainder of the study period (FIG. 7). However, treatment efficacy required that the disease had become established. Control mice began to exhibit initial disease symptoms (EAE score of at least 1 (limp tail, no paraparesis)) between days 10 and 14. Relative to the control group, mice receiving treatment prior to development of disease (i.e., treatment at days 6-10) exhibited a more pronounced disease course.

6.3 Anti-TCR Monoclonal Antibody Therapy in a Murine Model of Autoimmune Disease, Type-1 Diabetes

The efficacy of anti-TCR antibody for the treatment of autoimmune disorders was also assessed in a murine model of type-1 diabetes. Non-obese diabetic (NOD) mice exhibit a susceptibility to development of autoimmune insulin dependent diabetes mellitus as a result of spontaneous insulitis. Onset of diabetes in the NOD model is associated with a moderate glycosuria and a non-fasting hyperglycaemia. Administration of higher-dose anti-TCR antibody to diabetic NOD mice improved survival relative to the control group receiving no treatment.

Methods: Thirteen-week old female NOD mice were assessed for spontaneous development of diabetes by testing for gluscosuria. Urine glucose was tested on Tuesdays or Wednesdays, with mice having a positive reading re-tested on Thursday. All mice were also monitored by testing fasted serum glucose levels on Fridays. Mice with two positive urine readings in one week were isolated and assigned to experimental groups.

Isolated mice identified to have developed diabetes were randomized into treatment groups and injected i.v. with 50 μg (n=8) or 100 μg (n=4) of H57 AA per day, or with PBS (control/no treatment, n=10) over the course of 5 days.

Results: Treatment with 100 μg H57 AA per day over 5 days improved the survival of diabetic mice relative to the control group receiving no treatment. Treatment with 50 μg H57 AA per day over 5 days did not improve survival relative to control.

6.4 Anti-TCR Monoclonal Antibody Therapy for Patients with Autoimmune Diabetes

Patients: Forty patients with Type 1 diabetes are recruited for participation according to the following criteria: between 7 and 20 years of age, within 6 weeks of diagnosis according to the American Diabetes Association criteria, and confirmation of the presence of anti-GAD65, anti-ICA512, and/or anti-insulin autoantibodies. The patients remain under the care of their personal physicians during the course of the study.

Eligible patients are randomly assigned to a control group and a humanized anti-TCR antibody treatment group. After randomization, blood samples are drawn to establish baseline HA1c levels, a pretreatment C-peptide response to a MMTT is established and a pretreatment FPIR to IGTT is performed. Patients in both groups are hospitalized to receive either a 6-day course treatment of the humanized anti-TCR monoclonal antibody (ala-ala) or placebo. The antibody is administered intravenously in the following dosage: 17 μg/m² on day 1, 34.3 μg/m² on day 2, 69 μg/m² on day 3, 137.6 μg/m² on day 4, and 275.3 μg/m² on days 5 and 6. Alternatively, antibody may be administered intravenously in the following dosage: 1.6 μg/kg/day on day 1; 3.2 μg/kg/day on day 2; 6.5 μg/kg/day on day 3; 13 μg/kg/day on day 4; and 26 μg/kg/day on days 5 through 14. In dose escalation studies, the treatment may be, e.g., 1.42 μg/kg/day on day 1; 5.7 μg/kg/day on day 2; 11 μg/kg/day on day 3; 26 μg/kg/day on day 4; and 45.4 μg/kg/day on days 5 through 14. In subsequent studies, the therapy is altered to increase dosage and/or decrease the time course of treatment. For example, in subsequent studies patients may be administered a 4 day treatment: 6.4 μg/kg/day on day 1; 13 μg/kg/day on day 2, and 26 μg/kg/day on days 3 and 4; during additional dose escalation studies, the treatment may be 8 μg/kg/day on day 1; 16 μg/kg/day on day 2; and 32 μg/kg/day on days 3 and 4.

During initial studies the antibody dosage on the first three days of treatment is administered via slow infusion IV over 20 hours to monitor for adverse reactions. Subsequent studies will decrease the time of administration and/or split the dosage into 2 to 4 equal parts to be administered as bolus injections evenly distributed over the course of 12 hours. Patients in the control group undergo metabolic and immunologic tests but do no receive monoclonal antibodies. Patients are monitored throughout the study for immunosuppressive effects of the anti-TCR monoclonal antibody (ala-ala).

Patients are monitored for 18 months after the treatment. β-cell function is determined every 6 months in the case of impaired glucose tolerance and every 12 months in case of normal glucose tolerance. Patients are allowed to have a normal diet, and remain under the care of their personal physician throughout the duration of the study. Immunological assays are repeated in intervals of 6 months. Insulin therapy will be given to the patients as directed by their personal physician.

β-cell function will be analyzed according to the changes of the C-peptide levels as measured by radioimmunoassay. After drawing samples for baseline C-peptide and glucose, the patients are given a mixed meal. The C-peptide levels are measured in samples drawn after 15, 30, 60, 90, 120, 150, 180, 210, and 240 min. The C-peptide response to the mixed-meal tolerance test (MMTT) is expressed as the total area under the response curve (AUC). A change in the response is considered to have occurred if the response differs by more than 7.5 percent from the response at study entry. The patients' C-peptide responses to MMTT are continuously monitored 6 months, 9 months, 12 months, 15 months and 18 months after the treatment. Alternatively, the β-cell function is assessed by FPIR to IGTT. Serum insulin levels are measured by a modification of a double-antibody radioimmunoassay method using monoiodinated tyrosine A14-labeled insulin (Amersham Pharmacia). FPIR is calculated as the sum of insulin levels at 1 and 3 minutes after a glucose load (0.5 g/kg). Glycosylated hemoglobin levels are measured by latex-agglutination inhibition test.

Immunological Monitoring: The level of autoantibodies against GAD65, IA2/ICA512, and insulin are measured with radiobinding assays as known in the art (e.g., Woo et al., 2000, J. Immunol. Methods 244:91-103). HLA-DQA and HLA-DQB genotyping are performed by direct sequencing of exon 2 polymorphisims after PCR amplification. The level of cytokines in serum after the administration of the monoclonal antibody is measured by enzyme-linked immunosorbent assay (ELISA). Production of anti-idotype antibodies is monitored by ELISA assay using a plate bound anti-TCR (ala-ala) or by flow cytometry to measure blockade of binding of anti-TCR-FITC to the α chain of TCR.

Statistical Analysis: Data analysis will be conducted on residual beta-cell function, autoantibody level, cytokine level, and glycosylated hemoglobin level. A χ² analysis will be performed to test the effect of drug treatment before and after drug administration. Comparison between the control group and the treatment group will be made with the Mann-Whitney U test.

6.5 Anti-TCR Monoclonal Antibody Therapy in Subjects Predisposed to Type 1 Diabetes

Patients: Screening for subjects with predisposition for developing type 1 diabetes is based on first or second degree relationship with a diagnosed Type-1 diabetic; an impaired fasting glucose level; an impaired glucose response to OGTT; the presence of serum autoantibodies against GAD65, against IA2/ICA512, and/or against insulin; or impaired insulin production after MMTT, OGTT, IGTT or two phase glucose clamp procedure as determined by C-peptide response or FPIR. Patients who have been diagnosed with type 1 diabetes according to the criteria established by the American Diabetes Association by a physician, or who otherwise meet said criteria, are excluded from this study.

Patients selected for the study are randomly placed into two equal-sized groups. Treatment protocols and clinical monitoring are as described in section 6.1. Additionally, antibody therapy may be adjusted relative to residual β-cell function, i.e., patients with more impaired β-cell function as determined by C-peptide response or FPIR will receive a higher total dose of anti-TCR monoclonal antibody. For example, given two patients with C-peptide responses of 40 and 110 pmol/ml/240 min, the patient with impaired response will be given the higher of the two dosages tested, e.g., 1.42 μg/kg/day on day 1; 5.7 μg/kg/day on day 2; 11 μg/kg/day on day 3; 26 μg/kg/day on day 4; and 45.4 μg/kg/day on days 5 through 14.

Patients are monitored for 18 months after the treatment. β-cell function is determined every 6 months in the case of impaired glucose tolerance and every 12 months in case of normal glucose tolerance. Patients are allowed to have a normal diet, and remain under the care of their personal physician throughout the duration of the study. Immunological assays are repeated in intervals of 6 months. Insulin therapy will be given to the patients as directed by their personal physician.

6.6 Anti-TCR Monoclonal Antibody Therapy for the Treatment of T Cell Malignancy

To examine whether the effects of antibodies of the invention in the treatment of T cell malignancies, in vivo xenotransplant models of such malignancies may be established in SCID mice using, e.g., T cell lines, Raji Burkitt's lymphoma and/or Jurkat cells. For example, 1-2×10⁶ Raji or Jurkat cells may be subcutaneously injected in the flank of age and weight matched 8 week old mice to give rise to locally growing tumors. Treatment will begin when tumors reached a size of approximately 5 mm in diameter. The mice will be divided into four groups of five: treatment group, IV; treatment group IP, irrelevant antibody control, PBS. At days 0, 7, and 15 post tumor cell inoculation, each animal will receive 50 μl of PBS IV or IP containing 1 mg/kg anti-TCR antibody or irrelevant control. Tumor growth may be assessed according to the formula volume=(length×width²)/2. The animals are sacrificed when tumors reach the maximum tolerated size of 15 mm in diameter. Animals may be sacrificed at various time points to harvest tumors and determine tumor morphology.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed:

1. An isolated anti-TCR antibody comprising
a) a VH domain having the amino acid sequence of SEQ ID NO:2, SEQ ID NO:3, SEQ ID NO:4, SEQ ID NO:5 or SEQ ID NO:6; or
b) a VL domain having the amino acid sequence of SEQ ID NO:8, SEQ ID NO:9, SEQ ID NO:10, SEQ ID NO:11 or SEQ ID NO:12; or
c) a VH domain according to part a) and a VL domain according to part b).

2. The anti-TCR antibody according to claim 1 comprising a VH domain having the amino acid sequence of SEQ ID NO:6 and a VL domain having the amino acid sequence of SEQ ID NO:12.

3. The anti-TCR antibody according to claim 1 wherein said antibody
a) immunospecifically binds the extracellular constant domain of the TCR α chain; and/or
b) lacks an Fc region or comprises an Fc region that lacks effector function or has reduced effector function, and preferably wherein the amino acid corresponding to residue 297 of the Fc region is not asparagine; or
c) comprises an Fc region, wherein said Fc region is derived from human IgG1; or
d) is a single chain antibody; or
e) is a tetrameric antibody; or
f) has been chimerized or humanized.

4. A method of treating an autoimmune disorder or ameliorating the symptoms thereof in a patient in need thereof, said method comprising administering to said patient a therapeutically effective amount of the anti-TCR antibody of claim 1.

5. A method of treating a T-cell malignancy in a patient in need thereof, comprising administering to said patient a therapeutically effective amount of the anti-TCR antibody of claim 1.

6. The method according to claim 4, wherein said antibody lacks an Fc region or comprises an Fc region that lacks effector function or has reduced effector function.

7. The method according to claim 4, wherein said autoimmune disorder is associated with a deleterious inflammatory response, or wherein said autoimmune disorder is Type 1 diabetes, psoriasis, rheumatoid arthritis, lupus, inflammatory bowel disease (IBD), ulcerative colitis, Crohn's disease, multiple sclerosis, effects from organ transplantation, or graft vs. host disease (GVHD).

8. The method according to claim 4, wherein the anti-TCR antibody is administered a) at least once a day, preferably at least once a day over a period of at least 10 days; or b) once a week, twice a week, once every two weeks, once a month, once every six weeks, once every two months, twice a year or once a year.

9. The method according to claim 4, wherein the anti-TCR antibody is administered topically, orally, intravenously, intradermally, or subcutaneously.

10. The method according to claim 4, wherein the anti-TCR antibody is administered intravenously over at least one hour, or over at least 30 minutes, or over at least 15 minutes.

11. The anti-TCR antibody according to claim 1, wherein said antibody competes for binding with BMA 031 to the α-chain of TCR.

12. The method according to claim 4, wherein six months after said administration, said patient requires no increase in adjunctive therapy to manage said autoimmune disorder.

13. The method according to claim 4, wherein said patient is in the early stages of said autoimmune disease.

14. An isolated nucleic acid molecule comprising a) a nucleic acid sequence encoding the heavy chain variable domain, or a fragment thereof, of the antibody of claim 1a); or b) a nucleic acid sequence encoding the light chain variable domain, or a fragment thereof, of the antibody of claim 1b).

15. A vector comprising the nucleic acid molecule of claim 14.

16. A vector comprising a first nucleic acid molecule encoding a heavy chain and a second nucleic acid molecule encoding a light chain, said heavy chain and light chain being of the antibody or fragment thereof of claim 1c).

17. The vector of claim 15 which is an expression vector.

18. A host cell comprising the expression vector of claim 16.

19. A host cell containing a first nucleic acid operably linked to a heterologous promoter and a second nucleic acid operably linked to the same or a different heterologous promoter, said first nucleic acid and second nucleic acid encoding a heavy chain and a light chain, respectively, of the antibody of claim 1c).

20. A method for recombinantly producing a TCR specific antibody, said method comprising: (i) culturing in a medium the host cell of claim 18 under conditions suitable for the expression of said antibody; and (ii) recovery of said antibody from said medium.


SEQUENCE LISTING (19 sequences)

SEQ ID NO: 1 - protein, 120 aa, Artificial Sequence
  Mus
EVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSA

SEQ ID NO: 2 - protein, 120 aa, Artificial Sequence
  synthetic
QVQLVQSGAEVKKPGSSVKVSCKASGGTFSSYVMHWVRQAPGQGLEWMGYINPYNDVTKYNEKFKGRVTITADESTNTAYMELSSLRSEDTAFYFCAGGSYYDYDGFVYWGQGTLVTVSS

SEQ ID NO: 3 - protein, 120 aa, Artificial Sequence
  synthetic
QVQLVQSGAEVKKPGSSVKVSCKASGYKFTSYVMHWVRQAPGQGLEWMGYINPYNDVTKYNEKFKGRATITADESTNTAYMELSSLRSEDTAFYFCARGSYYDYDGFVYWGQGTLVTVSS

SEQ ID NO: 4 - protein, 120 aa, Artificial Sequence
  synthetic
QVQLVQSGAEVKKPGSSVKVSCKASGYKFTSYVMHWVKQAPGQGLEWIGYINPYNDVTKYNEKFKGKATLTADESTNTAYMELSSLRSEDTAVHYCARGSYYDYDGFVYWGQGTLVTVSS

SEQ ID NO: 5 - protein, 120 aa, Artificial Sequence
  synthetic
QVQLVQSGAEVKKPGSSVKVSCKASGYKFTSYVMHWVKQKPGQGLEWIGYINPYNDVTKYNEKFKGKATLTSDKSTSTAYMELSSLTSEDTAVHYCARGSYYDYDGFVYWGQGTLVTVSS

SEQ ID NO: 6 - protein, 120 aa, Artificial Sequence
  synthetic
QVQLVQSGAEVKKPGASVKVSCKASGYKFTSYVMHWVRQAPGQGLEWIGYINPYNDVTKYNEKFKGRVTITADKSTSTAYMELSSLRSEDTAVHYCARGSYYDYDGFVYWGQGTLVTVSS

SEQ ID NO: 7 - protein, 106 aa, Artificial Sequence
  mus
QIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELK

SEQ ID NO: 8 - protein, 106 aa, Artificial Sequence
  synthetic
DIQMTQSPSTLSASVGDRVTITCSATSSVSYMHWYQQKPGKAPKLLMYDTSKLASGVPSRFIGSGSGTEFTLTISSLQPDDFATYYCQQWSSNPLTFGGGTKVEIK

SEQ ID NO: 9 - protein, 106 aa, Artificial Sequence
  synthetic
DIQMTQSPSTLSASVGDRVTITCSATSSVSYMHWYQQKPGKAPKRLMYDTSKLASGVPARFIGSGSGTEFTLTISSLQPDDFATYYCQQWSSNPLTFGGGTKVEIK

SEQ ID NO: 10 - protein, 106 aa, Artificial Sequence
  synthetic
DIQMTQSPSTLSASVGDRVTMTCSATSSVSYMHWYQQKPGKAPKRWIYDTSKLASGVPARFIGSGSGTEFTLTISSLQPDDFATYYCQQWSSNPLTFGGGTKVEIK

SEQ ID NO: 11 - protein, 106 aa, Artificial Sequence
  synthetic
QIVLTQSPSTLSASVGDRVTITCSATSSVSYMHWYQQKPGTAPKRWIYDTSKLASGVPSRFSGSGSGTSYTLTISSLQPEDFATYYCQQWSSNPLTFGAGTKVEIK

SEQ ID NO: 12 - protein, 106 aa, Artificial Sequence
  synthetic
EIVLTQSPATLSLSPGERATLSCSATSSVSYMHWYQQKPGKAPKRWIYDTSKLASGVPSRFSGSGSGTEFTLTISSLQPEDFATYYCQQWSSNPLTFGQGTKLEIK

SEQ ID NO: 13 - protein, 13 aa, Artificial Sequence
  mus
KASGYKFTSYVMH

SEQ ID NO: 14 - protein, 13 aa, Artificial Sequence
  synthetic
KASGGTFSSYVMH

SEQ ID NO: 15 - protein, 17 aa, Artificial Sequence
  mus
YINPYNDVTKYNEKFKG

SEQ ID NO: 16 - protein, 11 aa, Artificial Sequence
  mus
GSYYDYDGFVY

SEQ ID NO: 17 - protein, 10 aa, Artificial Sequence
  mus
SATSSVSYMH

SEQ ID NO: 18 - protein, 7 aa, Artificial Sequence
  mus
DTSKLAS

SEQ ID NO: 19 - protein, 9 aa, Artificial Sequence
  mus
QQWSSNPLT